(19) United States
(12) Patent Application Publication
(10) Pub. No.: US 2024/0287015 A1
(43) Pub. Date: Aug. 29, 2024

(54) Method for Producing an Ionizable Lipid

(71) Applicant: NanoVation Therapeutics Inc., Vancouver (CA)

(72) Inventors: Marco A. Ciufolini, Vancouver (CA); Fariba Saadati, Vancouver (CA); Anthony C. Y. Tam, Vancouver (CA); Daniel Kurek, Vancouver (CA); Dominik Witzigmann, Binzen (DE); Jayesh Kulkarni, Vancouver (CA)

(73) Assignee: NanoVation Therapeutics Inc., Vancouver, BC (CA)

(21) Appl. No.: 18/564,780

(22) PCT Filed: May 26, 2022
(86) PCT No.: PCT/CA2022/050835

§ 371 Date: Nov. 28, 2023

Related U.S. Application Data
(60) Provisional application No. 63/194,471, filed on May 28, 2021.
(60) Provisional application No. 63/214,977, filed on Jun. 25, 2021.
(60) Provisional application No. 63/214,995, filed on Jun. 25, 2021.

(51) Int. Cl. C07D 317/28; A61K 9/51; C07C 29/143; C07C 45/65; C07C 227/18; C07C 319/20

(52) U.S. Cl. CPC C07D 317/28; A61K 9/5123; A61K 9/5192; C07C 29/143; C07C 45/65; C07C 227/18; C07C 319/20

(57) ABSTRACT

Provided herein is a method for producing an ionizable lipid that comprises: (i) reacting fatty esters in a Claisen condensation reaction in the presence of a catalyst, the Claisen condensation employing a weak base and carried out at a temperature of between −10 and 60 degrees Celsius to produce a ketoester; (ii) reacting the ketoester produced in step (i) under conditions to produce a ketone from the ketoester in one or more steps via a hydrolysis and decarboxylation of the ketoester; and (iii) preparing the ionizable lipid from the ketone thereof using one or more synthesis steps resulting in an addition of an ionizable head group moiety to (a) the ketone; or (b) an alcohol produced from an optional reduction of the ketone to produce the alcohol, thereby producing the ionizable lipid. The ionizable lipid produced in step (iii) may be formulated in a drug delivery vehicle.

### TECHNICAL FIELD

Provided herein is a method for producing ionizable lipids. The ionizable lipids may be formulated in a delivery vehicle so as to facilitate the incorporation of a wide range of therapeutic agents or prodrugs therein, such as, without limitation, nucleic acids (e.g., RNA or DNA), proteins, peptides and pharmaceutical drugs and salts thereof.

### BACKGROUND

Nucleic acid-based therapeutics have enormous potential in medicine. To realize this potential, however, the nucleic acid must be delivered to a target site in a patient. This presents challenges since nucleic acid is rapidly degraded by enzymes in the plasma upon administration. Even if the nucleic acid is delivered to a disease site, there still remains the challenge of intracellular delivery. To address these problems, lipid nanoparticles have been developed that protect nucleic acid from such degradation and facilitate delivery across cellular membranes to gain access to the intracellular compartment, where the relevant translation machinery resides.

A key component of lipid nanoparticles is an ionizable lipid. The ionizable lipid is typically positively charged at low pH, which facilitates association with the negatively charged nucleic acid. However, the ionizable lipid is neutral at physiological pH, making it more biocompatible in biological systems. Further, it has been suggested that after the lipid nanoparticles are taken up by a cell by endocytosis, the ionizability of these lipids at low pH enables endosomal escape. This in turn enables the nucleic acid to be released into the intracellular compartment.

Indeed, mRNA vaccines, including the covid19 Pfizer/BioNTech vaccine, rely on lipid nanoparticles to deliver mRNA to the cytoplasm of host cells. After entry into the host cell, the mRNA is transcribed to produce antigenic proteins. In the case of the covid19 vaccine, the mRNA encodes the Sars-Cov-2 spike protein. The ionizable lipid in the Pfizer/BioNTech is referred to as “ALC-0315” has a hydroxyl head group and a nitrogen atom that serves as anchoring point for branched lipid moieties.

An earlier example of a lipid nanoparticle product approved for clinical use and reliant on ionizable lipid is Onpattro®, developed by Alnylam. Onpattro® is a lipid nanoparticle-based short interfering RNA (siRNA) drug for the treatment of polyneuropathies induced by hereditary transthyretin amyloidosis. Onpattro® is reliant on an ionizable lipid referred to as “DLin-MC3-DMA” or more commonly “MC3” by investigators. This lipid has an ionizable dimethylamino head group, an ester linker and two C18 moieties derived from linoleic acid that converge into a single carbon atom. A related ionizable lipid, referred to by investigators as “KC2” also has a dimethylamino head group and two C18 moieties derived from linoleic acid, similarly converging into a single carbon atom, but the linker region comprises a 5 membered-ring with two oxygen atoms instead (a structure known by the person skilled in the art as a ketal). MC3 is a state-of-the art ionizable lipid and has been found to require about 3 times less siRNA than KC2, although KC2 remains a valuable research tool.

While the foregoing ionizable lipids have proven efficacious, there remains an ongoing need to expand the repertoire of ionizable lipids available for the formulation of new therapeutic agents or prodrugs in a wider range of applications.

Further, limited attention has been given to developing efficient and cost-effective synthesis routes to make ionizable lipid. Ionizable lipids currently require multi-step, complex reaction schemes using hazardous chemicals, adding cost and complexity to their manufacture. For example, the synthesis of MC3 requires six steps from methyl linoleate. As discussed in more detail further herein, this entails the preparation of an “MC3 alcohol” intermediate by a multiple step synthesis that includes the reduction of methyl linoleate with lithium aluminum hydride (LAH) and the elaboration of the resulting alcohol into a Grignard reagent. Steps that require LAH and Grignard reagents are routinely carried out in pharmaceutical plants, even though they are known to pose a fire hazard. The foregoing Grignard reagent reacts further to produce a synthetic intermediate that is described as “MC3 alcohol.” The latter is the coupled to an appropriate dimethylamino acid to furnish the desired MC3 (vide infra).

The synthesis of KC2 also involves the preparation of MC3 alcohol, but is followed by four additional steps to make the KC2 lipid, requiring a total of nine steps for its synthesis using current methods. One of these steps is the oxidation of MC3 alcohol with pyridinium chlorochromate (PCC). PCC is a problematic chemical reagent based on hexavalent chromium, which is a known carcinogen.

A more cost-effective and safer manufacturing method for ionizable lipids thus remains an unmet need in the industry.

The present disclosure seeks to address the shortcomings in the art and/or to provide useful alternatives to known methods for producing ionizable lipids.

### Definitions

The following terms have the meanings ascribed to them unless specified otherwise.

As used herein, the term “fatty esters” refers to chemical structures of the type shown as Formula I below, wherein R¹ is a linear or branched alkyl group having from 4 to 30 carbon atoms, and wherein the alkyl group may incorporate (i) from 0 to 4 heteroatoms, such as sulfur or oxygen atoms, (ii) from 0 to 5 C═C double bonds of E or Z geometry, and/or (iii) substituents such as OH, O-alkyl, S-alkyl, and N(alkyl)₂ bonded to a carbon atom thereof. R′ is an alkyl group having up to 5 carbon atoms, such as a methyl or ethyl group, or a glycerol residue that forms part of a larger molecule, such as a triglyceride, including olive oil, grapeseed oil, linseed oil, castor oil, tallow, and the like.

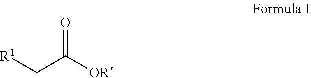

As used herein, the term “ketoester” refers to a chemical structure of the type shown as Formula II below, wherein the keto carbonyl and the ester carbonyl are in a 1,3 relationship (see numerical indices 1, 2, and 3), and R¹ and R² are linear or branched alkyl groups having from 4 to 30 carbon atoms, and wherein the alkyl groups may incorporate (i) from 0 to 4 heteroatoms, such as sulfur or oxygen atoms, (ii) from 0 to 5 C═C double bonds of E or Z geometry, and/or (iii) substituents such as OH, O-alkyl, S-alkyl, and N(alkyl)₂ bonded to a carbon atom. R³ may be H or a linear or branched alkyl group having from 4 to 30 carbon atoms, that may incorporate (i) from 0 to 4 heteroatoms, such as sulfur or oxygen atoms, (ii) from 0 to 5 C═C double bonds of E or Z geometry, and/or (iii) substituents such as OH, O-alkyl, S-alkyl, and N(alkyl)₂ bonded to a carbon atom. R′ is an alkyl group having up to 5 carbon atoms, such as a methyl or ethyl group, or a glycerol residue that forms part of a larger molecule, such as a triglyceride, including olive oil, grapeseed oil, linseed oil, tallow, and the like.

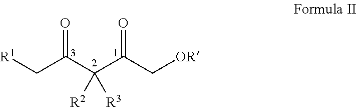

As used herein, the term “ketone” refers to a chemical structure of the type shown as Formula III below, wherein R¹ and R² are linear or branched alkyl groups having from 4 to 30 carbon atoms, and wherein the alkyl group may incorporate (i) from 0 to 4 heteroatoms, such as sulfur or oxygen atoms, (ii) from 0 to 5 C═C double bonds of E or Z geometry, and/or (iii) substituents such as OH, O-alkyl, S-alkyl, and N(alkyl)₂ attached to a carbon atom. R³ may be H, or a linear or branched alkyl group having from 4 to 30 carbon atoms, that may incorporate (i) from 0 to 4 heteroatoms, such as sulfur or oxygen atoms, (ii) from 0 to 5 C═C double bonds of E or Z geometry, and/or (iii) substituents such as OH, O-alkyl, S-alkyl, and N(alkyl)₂ attached to a carbon atom.

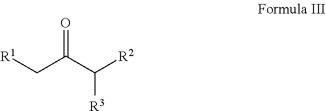

As used herein, the term “alcohol” refers to a chemical structure of the type shown as Formula IV below, wherein R¹ and R² are linear or branched alkyl groups having from 4 to 30 carbon atoms, that may incorporate (i) from 0 to 4 heteroatoms, such as sulfur or oxygen atoms, (ii) from 0 to 5 C═C double bonds of E or Z geometry, (iii) substituents such as OH, O-alkyl, S-alkyl, and N(alkyl)₂ bonded to a carbon atom. R³ may be H or a linear or branched alkyl group having from 4 to 30 carbon atoms, and wherein the alkyl group may incorporate (i) from 0 to 4 heteroatoms, such as sulfur or oxygen atoms, (ii) from 0 to 5 C═C double bonds of E or Z geometry, and/or (iii) substituents such as OH, O-alkyl, S-alkyl, and N(alkyl)₂ bonded to a carbon atom.

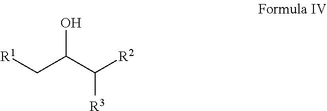

As used herein, the term “weak base” refers to a chemical species suitable for use in a given reaction step of the method described herein and which is capable of accepting a proton when placed in a solution, thereby producing a protonated form of itself, and such that the negative logarithm in base 10 of the aqueous ionization constant of said protonated form (i.e., its pKa) is between 4 and 13.

As used herein, the term “strong base” refers to a chemical species suitable for use in a given reaction step of the method described herein and which is capable of accepting a proton when placed in a solution, thereby producing a protonated form thereof, and such that the negative logarithm in base 10 of the aqueous ionization constant of said protonated form (i.e., its pKa) is greater than 13.

As used herein, the term “strong acid” refers to a chemical species suitable for use in a given reaction step of the method described herein and which is capable of donating a proton when placed in a solution, and such that the negative logarithm in base 10 of the aqueous ionization constant of said strong acid (i.e., its pKa) is lower than 3.

As used herein, the term “catalyst” refers to a chemical species that accelerates a reaction in a step of the method described herein, but that is not consumed in the course thereof. A catalyst thus allows the reaction to occur at a faster rate at lower temperatures.

As used herein, the term “ionizable lipid” refers to a lipid that, at a given pH, is in an electrostatically neutral form and that may either accept or donate protons, thereby becoming electrostatically charged, and for which the electrostatically neutral form has a calculated logarithm of the partition coefficient between water and 1-octanol (i.e., a cLogP) greater than 8.

As used herein, the term “ionizable head group moiety”, means a moiety that when incorporated within the ionizable lipid has at least one functional group that is capable of acquiring a net electrostatic charge, thereby becoming charged.

As used herein, the term “helper lipid” means a compound selected from: a sterol such as cholesterol or a derivative thereof; a diacylglycerol or a derivative thereof, such as a glycerophospholipid, including phosphatidic acid (phosphatidate) (PA), phosphatidylethanolamine (cephalin) (PE), phosphatidylcholine (PC), phosphatidylserine (PS), and the like; and a sphingolipid—such as a ceramide, a sphingomyelin, a cerebroside, a ganglioside—or reduced analogues thereof that lack a double bond in the sphingosine unit. The term encompasses lipids that are either naturally-occurring or synthetic.

As used herein, the term “nanoparticle” is any suitable particle in which an ionizable lipid can be formulated and that may comprise one or more helper lipid components. The one or more lipid components may include an ionizable lipid prepared by the method described herein and/or may include additional lipid components, such as a helper lipid. The term includes, but is not limited to, vesicles with one or more bilayers, including multilamellar vesicles, unilamellar vesicles and vesicles with an electron-dense core. The term also includes polymer-lipid hybrids, including particles in which the ionizable lipid is attached to a polymer.

### SUMMARY

The present disclosure provides a method for the preparation of various ionizable lipids. Such lipids may be capable of formulation in a delivery vehicle. Advantages of the synthesis schemes of the present disclosure include fewer method steps than conventional methods and/or method steps that avoid or reduce the use of hazardous chemicals. Advantageously, the disclosed method further enables the preparation of intermediates that serve as building blocks for the assembly of a variety of classes of new lipids.

In one embodiment, the present disclosure employs a variation of a Claisen condensation to produce a ketoester, which in turn is used to produce a ketone or alcohol to prepare a variety of lipids under milder and safer conditions than using conventional methods and/or with fewer reaction steps.

According to one aspect of the disclosure, there is provided a method for producing an ionizable lipid, the method comprising: (i) reacting fatty acid esters (“fatty esters” as defined herein) in a Claisen condensation reaction employing a weak base and at a temperature of between−10 and 60 degrees Celsius to produce a ketoester; (ii) reacting the ketoester produced in step (i) under conditions to produce a ketone from the ketoester in one or more steps via a hydrolysis and decarboxylation of the ketoester; and (iii) preparing the ionizable lipid from the ketone thereof using one or more synthesis steps resulting in an addition of an ionizable head group moiety to (a) the ketone; or (b) an alcohol produced from an optional reduction of the ketone to produce the alcohol, thereby producing the ionizable lipid.

According to a further aspect of the disclosure, there is provided a method for preparing a delivery vehicle comprising formulating the ionizable lipid produced in step (iii) in the delivery vehicle. The delivery vehicle may be a lipid nanoparticle, a liposome, or a lipoplex.

In one embodiment, the step of formulating comprises admixing a therapeutic agent or prodrug with the ionizable lipid to produce a delivery vehicle comprising same. The therapeutic agent or prodrug may include a nucleic acid, a pharmaceutical drug, a peptide or a protein.

According to one embodiment, the ketone is subjected to the reduction in step (iii) to produce the alcohol.

In another embodiment, the ketone is reduced to the alcohol by reacting the ketone with sodium borohydride.

In a further embodiment, the weak base in the Claisen condensation is an amine. For example, the amine may be a trialkylamine. In certain embodiments, the trialkylamine is tributylamine or triethylamine.

In one embodiment, the Claisen condensation comprises addition of AlCl₃, GaCl₃, TiCl₄, ZrCl₄, HfCl₄ or SnCl₄ as a catalyst. In a further embodiment, the Claisen condensation comprises an addition of TiCl₄ as a catalyst.

According to any one of the foregoing aspects or embodiments, the ketoester may be converted to the ketone with sequential base and acid additions. For example, the hydrolysis and decarboxylation may comprise reacting the ketoester with a strong base, and adding a strong acid to a resultant solution. In one embodiment, the strong base is selected from lithium hydroxide, sodium hydroxide, potassium hydroxide, calcium hydroxide, barium hydroxide and tetraalkylammonium hydroxides and the strong acid is selected from hydrochloric acid, sulfuric acid and phosphoric acid. In one embodiment, the strong base is aqueous sodium hydroxide and the strong acid is hydrochloric acid. Further, the hydrolysis and decarboxylation of the ketoester may comprise a step of heating.

In one embodiment, the acid of step (i) is obtained from a synthesis scheme comprising a step of ozonolysis to cleave a double bond in an alkyl group of a precursor fatty ester to produce an aldehyde derivative of the precursor fatty ester.

In another embodiment, the one or more steps resulting in an addition of an ionizable head group moiety to the ketone or alcohol comprises 1 to 5 steps.

According to any one of the foregoing aspects of embodiments, the ionizable lipid produced in step (iii) may comprise a linker region.

In a further embodiment, the fatty esters are methyl esters or ethyl esters. For example, the fatty esters may be methyl esters selected from methyl linoleate, methyl linolenate, methyl myristoleate, methyl palmitoleate, methyl myristate, methyl palmitate, methyl stearate, methyl 9-(((octylthio)methyl)thio)nonanoate and methyl 9,9-bis(octylthio)nonanoate.

In a further embodiment, the method further comprises the addition of an R³ alkyl group to the ketoester prior to the hydrolysis and decarboxylation of the ketoester.

Other objects, features, and advantages of the present disclosure will be apparent to those of skill in the art from the following detailed description and figures.

### BRIEF DESCRIPTION OF THE DRAWINGS

FIG. 1 depicts the synthesis scheme of one embodiment of the disclosure and the various ionizable lipids that can be generated therefrom.

### DETAILED DESCRIPTION

The present disclosure provides various lipid synthesis schemes to prepare an ionizable lipid. As those of ordinary skill in the art will appreciate, the reactions employed herein may be carried out in any appropriate solvent, or mixtures of solvents, and at appropriate temperatures.

The present disclosure is based on the finding that the use of a Claisen condensation step within a lipid synthesis scheme overcomes one or more obstacles associated with traditional synthesis schemes to make ionizable lipids for formulation in delivery vehicles. Lipid synthesis schemes using a step of Claisen condensation avoid the need for hazardous chemicals to produce a desired ionizable lipid and/or require fewer steps.

To illustrate, Comparative Example 1 describes the synthesis of new MC3 and KC2 derivatives, referred to herein as nor-MC3 and nor-KC2, using the synthesis of the present disclosure and sets forth the advantages of the inventive synthesis route using Claisen condensation over a conventional synthesis route to make MC3 and KC2. As demonstrated in this non-limiting example, the synthesis of ionizable lipids using the method described herein may avoid the use of lithium aluminum hydride (LAH), Grignard reagents and/or pyridinium chlorochromate (PCC) and eliminate a step or steps for preparing ketone or alcohols used as precursors for lipid synthesis.

The Synthesis Scheme shown below is an embodiment showing those steps of the synthesis scheme of the present disclosure that produce various intermediates for making ionizable lipids. The starting material for the synthesis in this example is a fatty acid alkyl ester, although the disclosure contemplates other starting materials, such as vegetable or animal oils or fats, such as olive oil, grapeseed oil, linseed oil, tallow, and the like, and mixtures of different fatty esters, as discussed below. As well, the fatty esters used as starting materials for the Claisen condensation reaction include other alkyl esters besides methyl esters, such as ethyl esters of fatty acids, and also encompasses fatty esters having R¹ and R² alkyl groups that are linear or branched with saturated chains, unsaturated chains and/or chains substituted with heteroatoms.

Referring to the example Synthesis Scheme below, Claisen condensation of a fatty acid methyl ester produces a ketoester 2. The ketoester 2 is optionally reacted with a suitable reagent to add an additional alkyl group R² using known synthesis methods to produce a ketoester having three alkyl groups (depicted here as R¹, R¹ and R²). The resultant ketoester 2 from the Claisen condensation (that is, either a two or three alkyl ketoester 2) is converted into a corresponding ketone 3 or 3a, which in turn is used to synthesize ionizable lipids according to the synthesis schemes set out below. The ketone 3 or 3a may be converted to an alcohol 4 or 4a, which may alternatively or additionally be used to synthesize a variety of ionizable lipid classes as described herein.

### Synthesis Scheme:

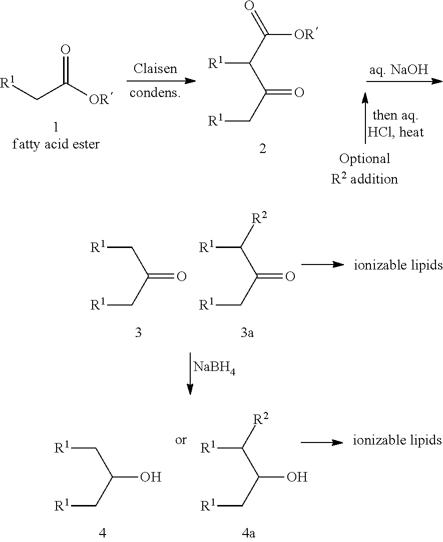

In the reaction scheme above, the R¹ groups, and the R² if present, independently may be a linear or branched alkyl group having from 4 to 30 carbon atoms, and wherein the alkyl groups may incorporate (i) from 0 to 4 heteroatoms, such as sulfur or oxygen atoms, (ii) from 0 to 5 C═C double bonds of E or Z geometry, and/or (iii) substituents such as OH, O-alkyl, S-alkyl, and N(alkyl)₂ bonded to a carbon atom. R′ may be H or a linear or branched alkyl group having from 4 to 30 carbon atoms, that may incorporate (i) from 0 to 4 heteroatoms, such as sulfur or oxygen atoms, (ii) from 0 to 5 C═C double bonds of E or Z geometry, and/or (iii) substituents such as OH, O-alkyl, S-alkyl, and N(alkyl)₂ bonded to a carbon atom. R′ is an alkyl group having up to 5 carbon atoms, such as a methyl or ethyl group, or a glycerol residue that forms part of a larger molecule, such as a triglyceride like olive oil, grapeseed oil, linseed oil, tallow, and the like.

For example, without intending to be limiting, in order to prepare new analogues of KC2 and MC3 having 17 carbon chains instead of 18 carbon chains (referred to herein as nor-KC2 and nor-MC3), methyl linoleate can be used as the fatty acid methyl ester 5 used as the starting material for the above general synthesis scheme. The illustrative example below depicts the conversion of methyl linoleate 5 into a corresponding ketoester 6 by a Claisen condensation, followed by the conversion of the ketoester 6 into ketone 7 via a hydrolysis and/or decarboxylation:

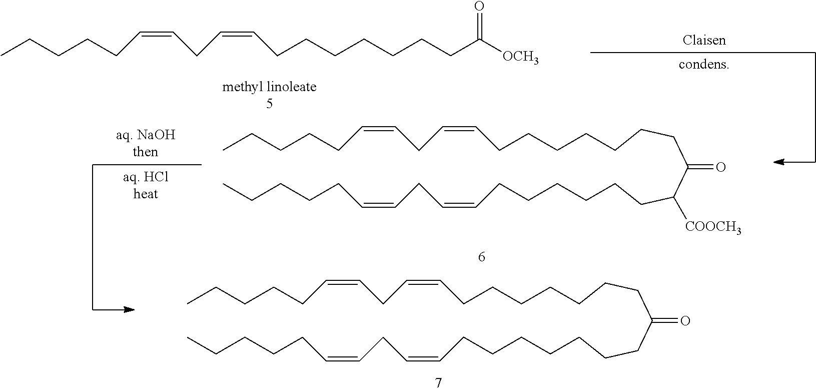 [SMILES: CCCCC/C=C\C/C=C\CCCCCCCC(=O)OC ; CCCCC/C=C\C/C=C\CCCCCCCC(=O)C(CCCCCC/C=C\C/C=C\CCCCC)C(=O)OC ; CCCCC/C=C\C/C=C\CCCCCCCC(=O)CCCCCCC/C=C\C/C=C\CCCCC]

The ketone 7 produced by the above synthesis scheme can be used as an intermediate to produce nor-KC2 and nor-MC3 lipids having the following structures:

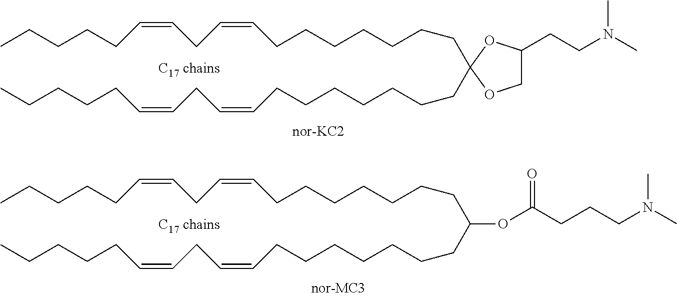

The synthesis schemes for producing nor-KC2 and nor-MC3 from the above ketone 7 are described in more detail in Scheme A and Scheme B below, respectively. As described in Scheme A below, nor-KC2 is produced from the ketone 7 above, while nor-MC3 (Scheme B) is prepared by converting ketone 7 to a corresponding alcohol 8 (using e.g., NaBH₄), which is shown below:

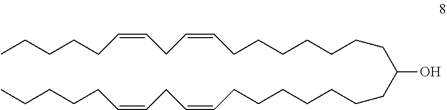 [SMILES: CCCCC/C=C\C/C=C\CCCCCCCC(O)CCCCCCC/C=C\C/C=C\CCCCC]

While the production of intermediates for preparing nor-KC2 and nor-MC3 lipids has been outlined above, the present disclosure is more broadly applicable to the synthesis of a wide variety of ionizable lipids, including entirely new classes of lipids, as described hereinafter.

### Starting Material for Claisen Condensation

The starting material for the Claisen condensation includes any suitable solution or preparation comprising one or more fatty ester as defined herein. The solution or preparation may comprise a mixture of different fatty esters or most advantageously comprise only one type of fatty ester.

It should be understood that the fatty ester used as a starting material for the Claisen condensation may be any molecule or compound produced from prior treatment of a fatty ester. Such preliminary treatment steps may be used to make fatty esters substituted with heteroatoms, such as sulfur atoms (e.g., methyl 9-(((octylthio)methyl)thio)nonanoate in Scheme G below) or to prepare branched sulfur fatty acids (e.g., methyl 9,9-bis(octylthio)nonanoate in Scheme I below) that are subsequently introduced as a starting material for the Claisen condensation step.

For example, such treatment steps to produce a starting material for Claisen condensation include ozonolysis of an unsaturated fatty ester (such as an unsaturated fatty acid methyl ester), followed by reduction of peroxide intermediates and additional synthesis steps to produce a fatty acid methyl ester having sulfur atoms in its R alkyl group (see e.g., Scheme G below).

Another non-limiting example includes treatment of a fatty ester, such as an oleate ester 9 (Scheme I), with O₃ followed by Zn/AcOH to produce an aldehydoester such as 10 followed by thioacetalization of the aldehyde with an appropriate thiol, (e.g., 1-octanethiol) in the presence of a suitable acid, such as H₂SO₄, leading to the formation of a fatty ester incorporating a thioacetal group, such as a 9,9-bis(octylthio)nonanoate ester such as 11. Ester 11 thus obtained is the starting material for a Claisen condensation that produces a ketoester such as 12, which may be converted into ketone 13, which is the precursor of a novel family of dendritic lipids (“dendripids”) via the synthetic route shown in Scheme I. It should be understood, however, that the foregoing are simply examples of preliminary treatment steps to produce a fatty ester that is introduced to the Claisen condensation as a starting material and should not be construed as limiting in any way.

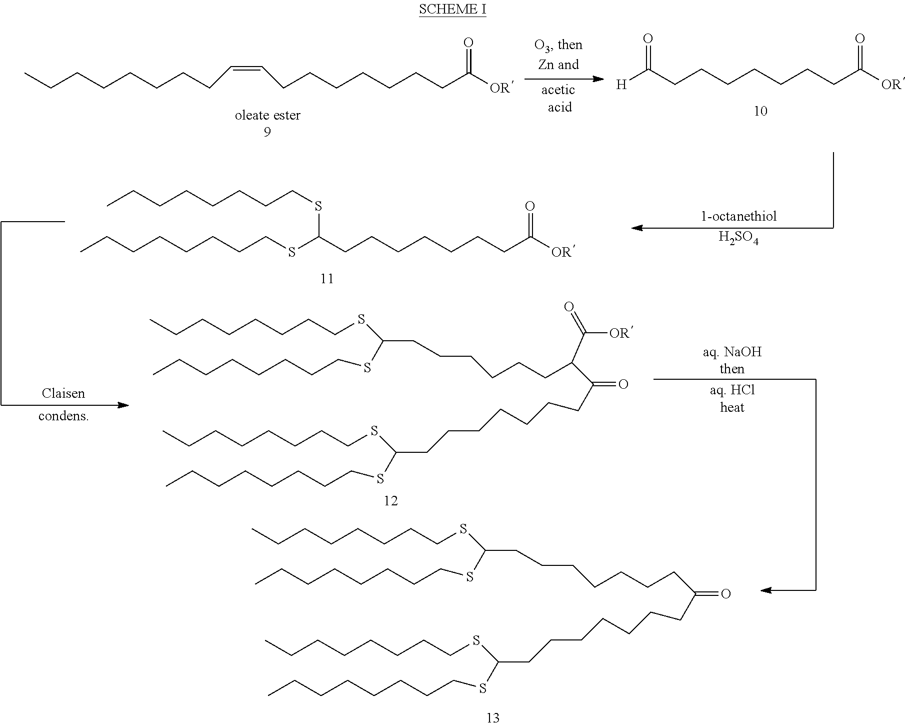

In one embodiment, the fatty ester has a chemical structure of the type shown below:

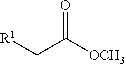

wherein R¹ is a linear or branched alkyl group having from 4 to 30 carbon atoms, and wherein the alkyl group may incorporate (i) from 0 to 4 heteroatoms, such as sulfur or oxygen atoms, (ii) from 0 to 5 C═C double bonds of E or Z geometry, and/or (iii) substituents such as OH, O-alkyl, S-alkyl, and N(alkyl)₂ bonded to a carbon atom thereof.

In one non-limiting embodiment, the fatty ester subjected to Claisen condensation is selected from a methyl linoleate, methyl oleate, methyl linolenate, methyl myristoleate, methyl palmitoleate, methyl myristate, methyl palmitate, methyl stearate, methyl 9-(((octylthio)methyl)thio)nonanoate and methyl 9,9-bis(octylthio)nonanoate.

In another non-limiting embodiment, the fatty ester subjected to Claisen condensation is selected from a suitable vegetable or animal oil or fat, such as olive oil, grapeseed oil, linseed oil, tallow, and the like.

In those embodiments in which a vegetable or animal oil or fat is used in the Claisen condensation, the vegetable or animal oil or fat, neat or diluted with an appropriate solvent, may be treated by passage through a solid chromatographic support, such as silica gel, alumina, florisil, and the like, prior to Claisen condensation. Such a treatment maybe useful to remove impurities and/or other unwanted components.

### Claisen Condensation of Fatty Esters, Oil or Fat

In one advantageous embodiment, the Claisen condensation used in the lipid synthesis is a milder variant of a more conventional Claisen condensation. Conventional Claisen condensations use strong bases such as sodium hydride or sodium alkoxides under elevated temperature conditions, such as greater than 100 degrees Celsius. However, such strong bases can be hazardous, particularly when reacted at high temperatures. Another advantage of using a milder Claisen condensation is that when unsaturated fatty acid esters are used as starting materials for lipid synthesis, the double bonds are less prone to isomerization under the milder conditions.

However, it will be understood that in those embodiments employing a fatty ester with a saturated chain, or only one double bond, a conventional Claisen reaction could be utilized rather than a milder variant. An example of a scheme that might employ a Claisen condensation with an alkoxide or other strong base and/or at high temperature is Scheme F described herein that uses methyl myristate, methyl palmitate and/or methyl stearate as a starting fatty acid. Other saturated fatty acids known to those of skill in the art could be used as starting materials for a Claisen condensation employing a strong base and/or high temperatures.

The Claisen condensation may comprise the addition of reagents that are considered weak bases, such as tertiary amines. Non-limiting examples of tertiary amines suitable for use in the Claisen condensation are trialkyl amines such as trimethylamine, triethylamine, tripropylamine, tributylamine, diisopropylethylamine, and the like. Triethylamine and tributylamine are preferred.

The Claisen condensation may be carried out at mild temperature conditions, such as between−10 and 60 degrees Celsius or between−10 and 55 degrees Celsius or between−10 and 50 degrees Celsius or between−10 and 40 degrees Celsius to produce a ketoester. Alternatively, the Claisen condensation may be carried out at mild temperature conditions, such as between−5 and 60 degrees Celsius or between−5 and 55 degrees Celsius or between−5 and 50 degrees Celsius or between−5 and 40 degrees Celsius to produce a ketoester. Alternatively, the Claisen condensation may be carried out at mild temperature conditions, such as between 0 and 60 degrees Celsius or between 0 and 55 degrees Celsius or between 0 and 50 degrees Celsius or between 0 and 40 degrees Celsius to produce a ketoester.

In another embodiment, the Claisen condensation is carried out at a temperature of less than 100 degrees Celsius, less than 80 degrees Celsius, less than 60 degrees Celsius, less than 55 degrees Celsius, less than 50 degrees Celsius or less than 45 degrees Celsius.

The Claisen condensation may be carried out in the presence of a catalyst. The catalyst is most advantageously a suitable metallic salt. Non-limiting examples of catalysts for the above reaction are aluminum trichloride (AlCl₃), gallium trichloride (GaCl₃), titanium tetrachloride (TiCl₄), zirconium tetrachloride (ZnCl₄), hafnium tetrachloride (HfCl₄), stannic chloride (SnCl₄). Titanium tetrachloride is a preferred catalyst for the Claisen condensation.

The ketoester 2 may subsequently be converted to the ketone 3, which in turn is used as a precursor to various synthetic schemes as described further herein.

It should also be understood, however, that the ketoester can be used to create an ionizable lipid via a synthetic scheme in which the ketoester is not converted directly to a corresponding ketone. Such a scheme comprises an intervening step to add an additional R² group to a ketoester having two alkyl chains R to produce a ketoester having three alkyl chains (see Scheme II below). Such ketoester having three alkyl groups can be converted to a corresponding ketone or alcohol having three alkyl groups to prepare ionizable lipids having three alkyl groups. Examples include branched analogues of MC3 or KC2 and NV1000 lipids described herein (Scheme K) having three R alkyl groups.

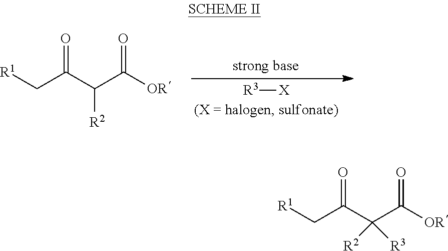

Without being limiting, the ketoester may be derived from a fatty ester that is a methyl ester.

Thus, according to one embodiment, the ketoester may be selected from a structure having one of the following general formulas:

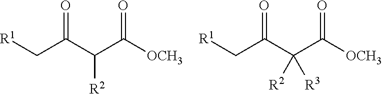

wherein R¹ and R² are linear or branched alkyl groups having from 4 to 30 carbon atoms, and wherein the alkyl group may incorporate (i) from 0 to 4 heteroatoms, such as sulfur or oxygen atoms, (ii) from 0 to 5 C═C double bonds of E or Z geometry, and/or (iii) substituents such as OH, O-alkyl, S-alkyl, and N(alkyl)₂ bonded to a carbon atom. R³ may be H or a linear or branched alkyl group having from 4 to 30 carbon atoms, that may incorporate (i) from 0 to 4 heteroatoms, such as sulfur or oxygen atoms, (ii) from 0 to 5 C═C double bonds of E or Z geometry, and/or (iii) substituents such as OH, O-alkyl, S-alkyl, and N(alkyl)₂ bonded to a carbon atom.

It should be appreciated, however, that the R′ alkyl group of the above ketoester may be selected from other alkyl groups besides a methyl group as per the general ketoester formula set forth above.

### Hydrolysis and Decarboxylation of the Ketoester to Produce the Ketone

The ketoester having two or three alkyl groups as defined herein is subsequently subjected to a hydrolysis and decarboxylation reaction to produce a corresponding ketone.

To produce the ketone 3, the ketoester can undergo hydrolysis and subsequent decarboxylation under basic or acidic conditions. Hydrolysis forms a keto acid, while decarboxylation of this acid produces carbon dioxide and the corresponding ketone 3.

Such hydrolysis and decarboxylation reaction may include the addition of an aqueous solution of a strong base followed by addition of aqueous solution of a strong acid followed by heating. As would be appreciated by those of skill in the art, a variety of different acids and bases could be utilized in the hydrolysis/decarboxylation to produce the corresponding ketone from the ketoester.

Non-limiting examples of such strong bases include metal hydroxides such as lithium hydroxide, sodium hydroxide, potassium hydroxide, calcium hydroxide, barium hydroxide, and the like; or tetraalkylammonium hydroxides such as tetramethylammonium hydroxide, tetraethylammonium hydroxide, tetrabutylammonium hydroxide, benzyltrimethylammonium hydroxide, and the like.

Non-limiting examples of such strong acids are hydrochloric acid, sulfuric acid, phosphoric acid, methanesulfonic acid, benzenesulfonic acid, toluenesulfonic acid, and the like.

Sodium hydroxide and hydrochloric acid are preferred.

As discussed, the inclusion of an optional step comprising adding an R³ group prior to or during the hydrolysis and decarboxylation of the ketoester having two R groups will produce a ketoester having an additional R³ group, thereby allowing for the preparation of branched ionizable lipid analogues having three alkyl groups (R¹, R² and R³). (See e.g., Scheme K below for preparing trialkyl analogues of ionizable lipids).

In some cases, it is expedient to convert a starting fatty ester into a corresponding ketone without isolation of the intermediate ketoester. This is often the case for O-silyl derivatives of hydroxylated fatty ester, such as ricinoleate esters. For example, methyl ricinoleate may be expediently converted into (7R,9Z,26Z,29R)-7,29-dihydroxypentatriaconta-9,26-dien-18-one through a synthetic sequence involving: (i)O-silylation of the starting methyl ricinoleate to produce methyl (R,Z)-12-((tert-butyldimethylsilyl)oxy)octadec-9-enoate; (ii) Claisen condensation of methyl (R,Z)-12-((tert-butyldimethylsilyl)oxy)octadec-9-enoate to produce methyl (14R,Z)-14-((tert-butyldimethylsilyl)oxy)-2-((R,Z)-10-((tert-butyldimethylsilyl)oxy) hexadec-7-en-1-yl)-3-oxoicos-11-enoate; base hydrolysis, decarboxylation, and and O-desilylation of the latter to produce (7R,9Z,26Z,29R)-7,29-dihydroxypentatriaconta-9,26-dien-18-one directly. Details are provided in the experimental section.

### Conversion of the Ketone to a Corresponding Alcohol

The ketone 3 above having two or three alkyl groups may be subsequently subjected to one or more steps comprising a reduction step to produce a corresponding alcohol.

The reagent for reducing the ketone may serve as a source of hydride that functions as a hydride nucleophile for the reduction. The addition of the hydride anion to the ketone produces an alkoxide anion, and a protonation results in the corresponding alcohol 4.

An example of a reagent that can be used in the reduction step is sodium borohydride (NaBH₄). The reagent, LiAlH₄ may be used as well if desired, although it can react violently with water, alcohols and acidic groups with evolution of hydrogen gas. Thus, in one embodiment, the reduction of the ketone to a corresponding alcohol does not include the addition of LiAlH₄.

As would be appreciated by those of skilled in the art, conversion of the ketone to the corresponding alcohol with the reducing agent is typically carried out in a suitable solvent. An alcohol solvent, such as methanol, ethanol, propanol, isopropanol, butanol, isobutanol, and the like, may be used in the reduction when the reduction is carried out with NaBH₄. Other suitable solvents known to those of skill in the art may be used when the reduction is carried out with reducing agents other than NaBH₄.

Producing the Ionizable Lipid from the Ketone or Alcohol

The method further comprises preparing an ionizable lipid from the ketone thereof using a synthesis scheme having one or more steps to add an ionizable head group moiety to one of:
(a) the ketone; or
(b) an alcohol produced from an optional reduction of the ketone.

The ionizable head group moiety may become positively charged, which facilitates association with the negatively charged nucleic acid. The ionizable head group moiety may be neutral at physiological pH, making the lipid more biocompatible in biological systems. However, the ionizable head group may become negatively charged for association with a positively charged cargo molecule. The ionizable head group moiety optionally comprises a linker region for linkage of the head group to the alkyl groups of the ionizable lipid.

### (a) Ketone

Non-limiting examples of synthesis steps that result in the addition of an ionizable moiety to a ketone to produce an ionizable lipid are shown below. It will be appreciated that the addition of an ionizable moiety to a ketone can be carried out with ease by those of ordinary skill in the art using conventional organic synthesis techniques. The below discussion illustrates the addition of a KC2 linker and ionizable head group moiety to the ketone. However, various head groups, such as ionizable moieties and linkers are known in the art and a suitable group or moiety for addition to the ketone can be selected by those of skill in the art as required. The addition of the ionizable head group to the ketone may comprise one or more steps.

The ketone 7 (C₁₇ alkyl groups with two double bonds in each alkyl group) having the structure below, can be converted to nor-KC2 in a synthesis comprising involving the addition of a triol, such as 1,2,4-butanetriol, with TsOH, in an appropriate solvent such as, but not limited to toluene. Subsequent steps of MsCl, Et₃N, CH₂Cl₂ addition and addition of Me₂NH produce the nor-KC2 ionizable lipid, as shown below:

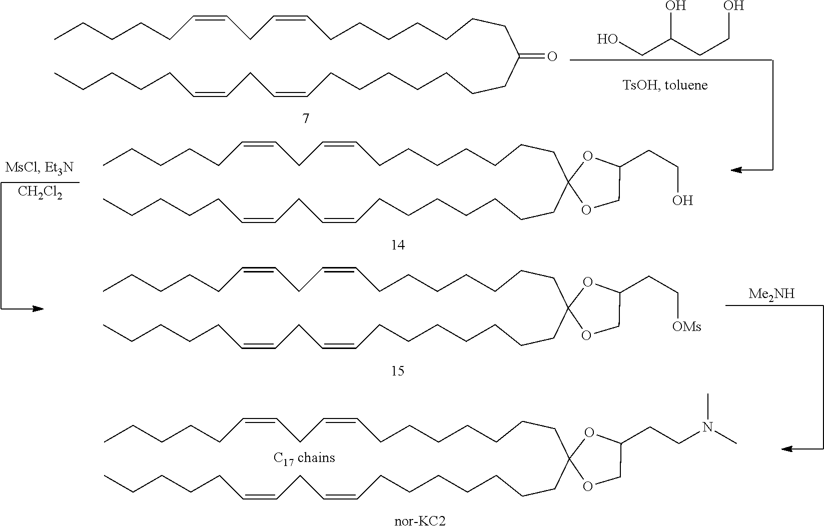

Alternatively, ketone 7 can be converted to nor-KC2 in a synthesis comprising ketalization with an aminodiol hydrochloride such as 16:

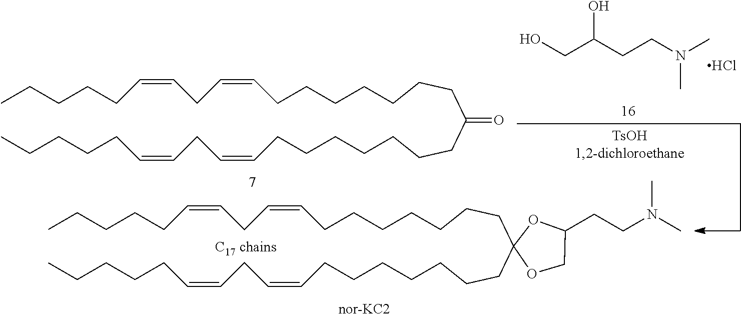

The reaction scheme to produce nor-KC2 from a fatty ester is shown below in more detail in Scheme A.

Similarly, a sulfur-containing ketone 17 having the structure below, as noted, can be converted to a KC2 sulfur analogue 20 in a sequence involving the addition of a triol, such as 1,2,4-butanetriol, with TsOH, and toluene, with subsequent steps of MsCl, Et₃N, CH₂Cl₂ addition and addition of Me₂NH to produce the nor-KC2 sulfur derivative:

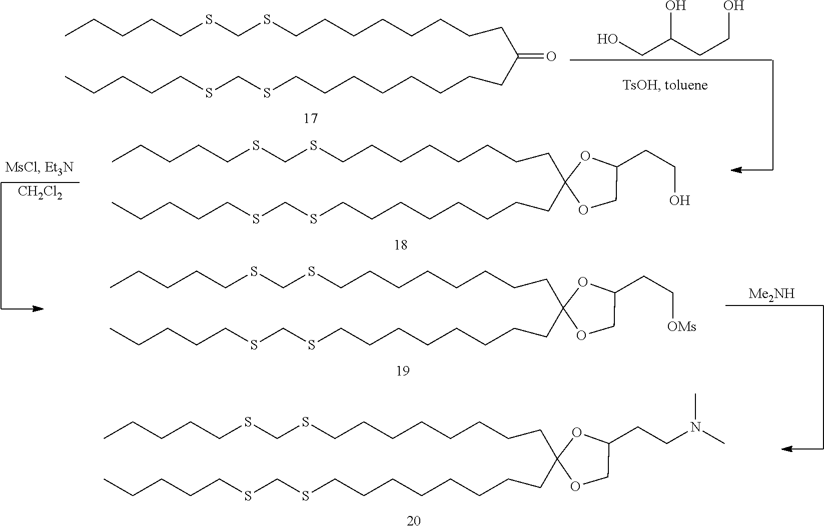

Alternatively, ketone 17 can be converted to compound 20 in a synthesis comprising ketalization with an aminodiol hydrochloride such as 16:

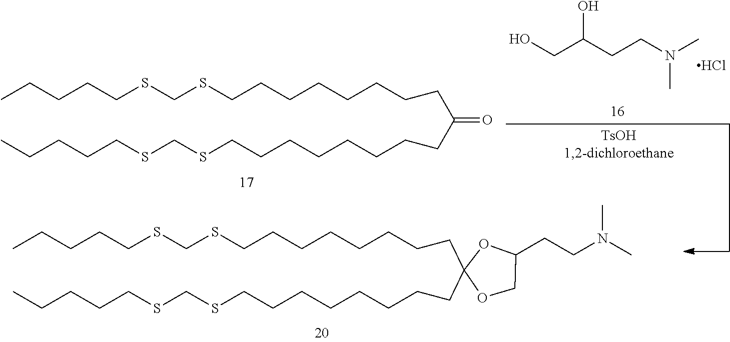

The reaction scheme to produce sulfur KC2 analogues from a fatty ester is shown below in more detail in Scheme H.

A branched sulfur-containing ketone such as 13 can be used to prepare a KC2-like sulfur lipid 21 using the same scheme as above involving the addition of a triol, such as 1,2,4-butanetriol, with TsOH, and toluene, with subsequent steps of MsCl, Et₃N, CH₂Cl₂ addition and addition of Me₂NH, or involving the addition of the hydrochloride of aminoalcohol such as 16, to produce the nor-KC2 dendripid structure:

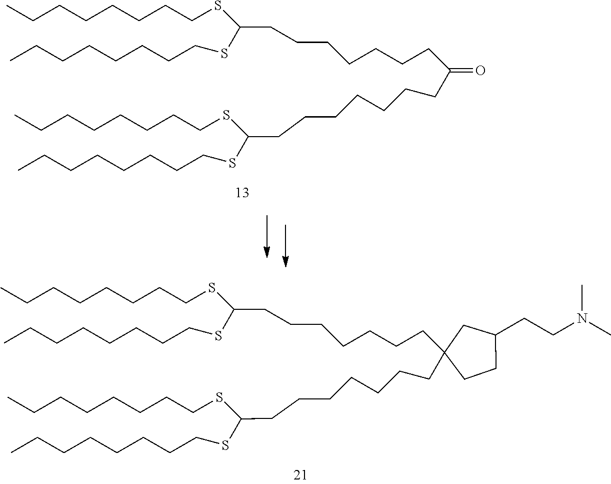 [SMILES: CCCCCCCCSC(CCCCCCCC(=O)CCCCCCCC(SCCCCCCCC)SCCCCCCCC)SCCCCCCCC ; CCCCCCCCSC(CCCCCCCC1(CCCCCCCC(SCCCCCCCC)SCCCCCCCC)CCC(CCN(C)C)C1)SCCCCCCCC]

While a variety of examples of synthesis schemes having one or more steps comprising addition of an ionizable moiety to the ketone 3 are described herein, those of ordinary skill in the art will appreciate that the schemes above are exemplary and that alternative schemes could be used to prepare an ionizable lipid from a ketone or such schemes may include steps in addition to those set out above. This may comprise, for example, a reaction scheme whereby an ionizable group is introduced through reductive amination of a ketone of the type 3 (see Scheme J below).

### (b) Alcohol

Non-limiting examples of synthesis steps that add an ionizable moiety to an alcohol 4 to produce an ionizable lipid are shown below. It will be appreciated that the addition of an ionizable moiety to an alcohol can be carried out with ease by those of ordinary skill in the art using conventional organic synthesis techniques. The below discussion illustrates the addition of an MC3 linker and ionizable moiety to the alcohol. However, various head groups, incorporating ionizable moieties and linkers are known in the art and a suitable group or moiety for addition to the alcohol can be selected by those of skill in the art as required.

The alcohol 8 (e.g., C₁₇ alkyl groups with two Z double bonds per group) having the structure below, may be converted to nor-MC3 by the following synthesis scheme:

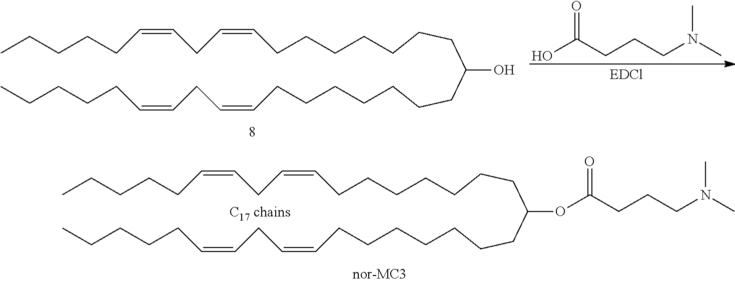

The alcohol 22 (e.g., C₁₇ chains with one Z double bond per chain) having the structure below, can be converted to an anionic ionizable lipid, 23, by the following synthesis scheme that includes addition of succinic anhydride:

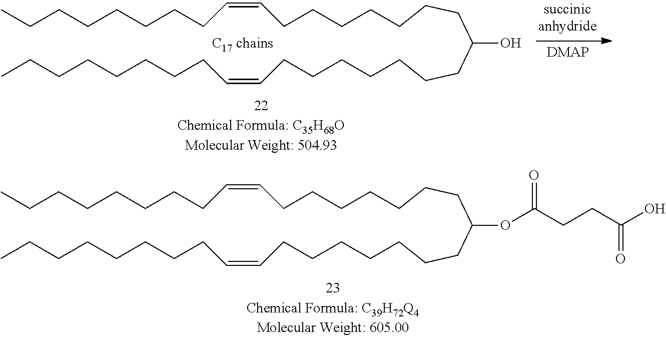 [SMILES: CCCCCCCC/C=C\CCCCCCCC(O)CCCCCCC/C=C\CCCCCCCC ; CCCCCCCC/C=C\CCCCCCCC(CCCCCCC/C=C\CCCCCCCC)OC(=O)CCC(=O)O]

It should be appreciated that the alcohol can undergo further modification prior to addition of an ionizable head group. For example, an alcohol can undergo the following reaction scheme involving ozone addition to produce an alcohol 25 that is branched.

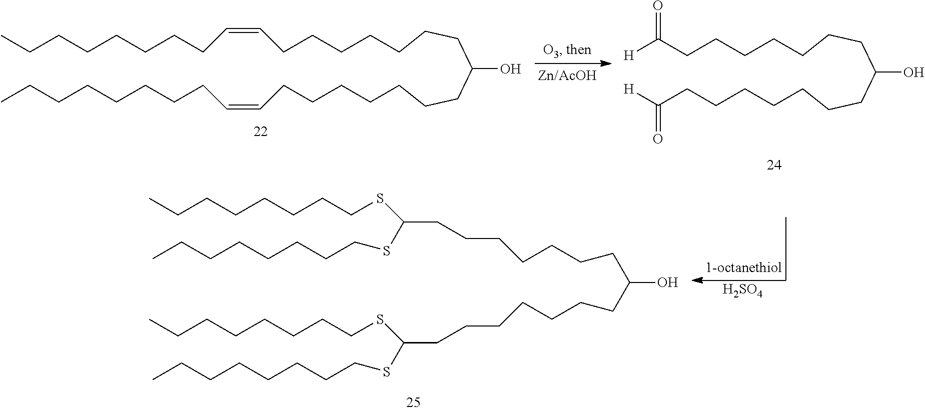

The branched alcohol 25 can be converted to the ionizable lipid 26 by another step comprising addition of the ionizable head group moiety:

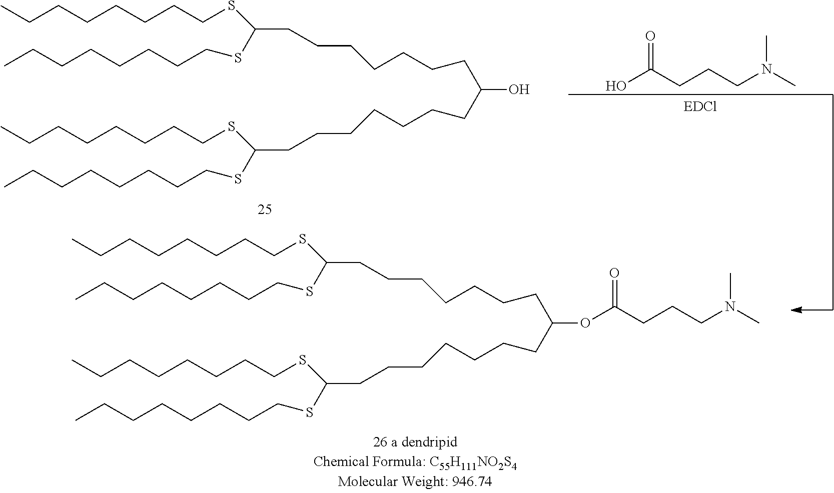

The resultant ionizable lipid is a branched lipid. A branched lipid is referred to herein as a “dendripid” and includes any ionizable lipid produced by the method of the disclosure that has one or more branched R¹ or R² alkyl groups. The synthesis of dendripids is described in more detail in Scheme D and non-limiting examples of dendripid structures are set forth in more detail in Structural Formula D and E below.

Further examples of the addition of a functional head group moiety to an alcohol are provided below:

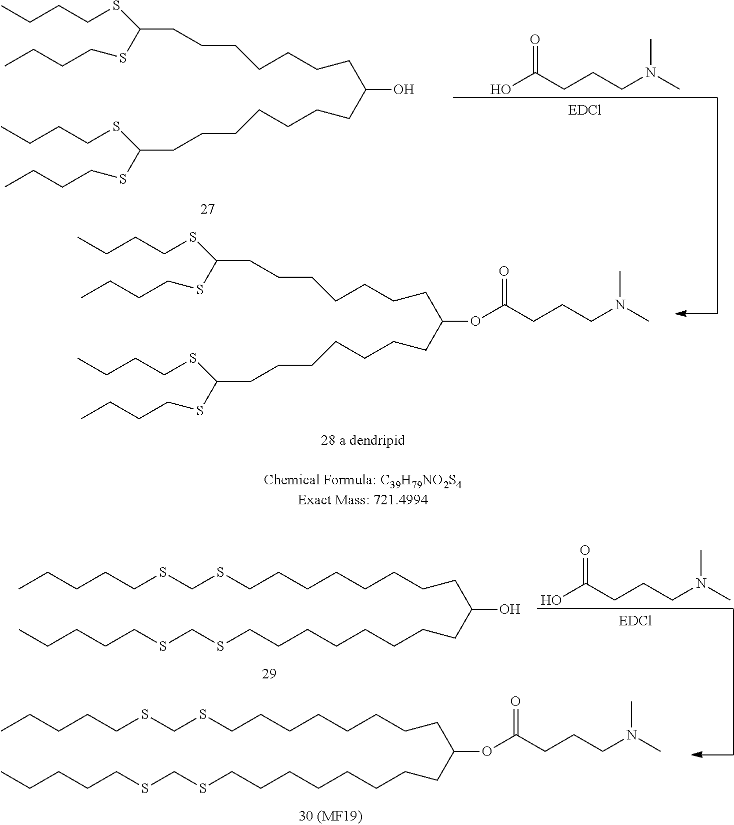

Lipid 30, described herein as MF19, is a sulfur-containing analogue of ionizable lipid MC3. The synthesis of such lipids is described in more detail in Scheme G and examples of sulfur lipid structures are set forth in more detail in Structural Formula F below.

Yet a further example of the addition of a functional head group moiety to an alcohol is the general scheme below to produce trialkyl lipids:

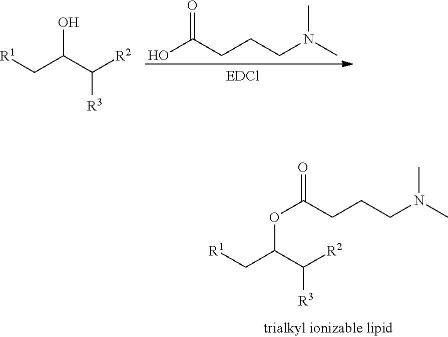

The synthesis of such ionizable lipids having three alkyl R¹, R², R³ groups (e.g., trialkyl lipids) is described in more detail in Scheme K below and examples of such ionizable lipids are set forth in more detail in Structural Formula A, B and C below.

Examples of Schemes for Preparing Ionizable Lipid from the Ketone or Alcohol

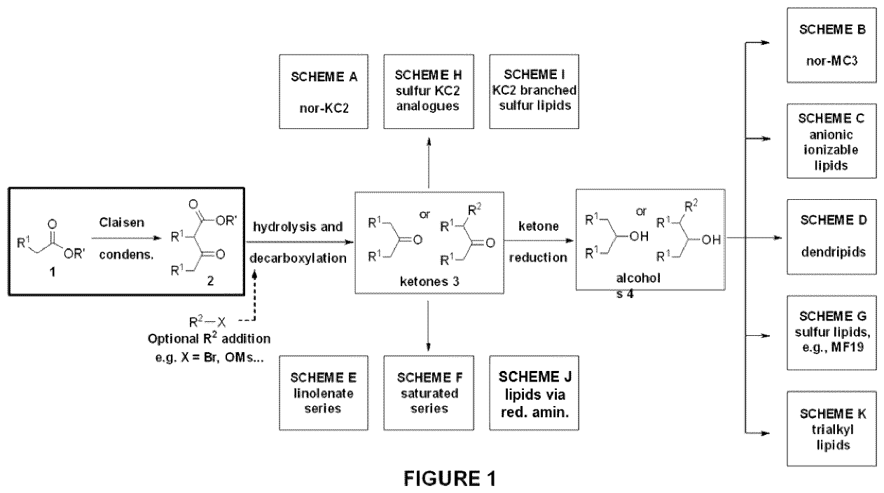

FIG. 1 provides an overview of examples of the various lipids and lipid classes that can be made using the various intermediates (ketoester, ketone or alcohol) resulting from a synthesis route using a step of Claisen condensation.

The reaction scheme of FIG. 1 uses a fatty acid methyl ester 1 as the starting material in this example. The fatty acid methyl ester 1 encompasses a wide range of different structures. The R¹, R², and R³ alkyl groups can be linear or branched alkyl groups, optionally substituted with one or more heteroatoms, such as S or 0, and having up to 30 carbon atoms. In addition, the R or R′ of the fatty acid can be saturated or have varying degrees of unsaturation. Moreover, the fatty acid methyl ester 1 can itself be a product of an organic synthesis, such as a synthesis comprising an upstream ozonolysis reaction (see e.g., Schemes G and I described below).

As further shown in FIG. 1, a Claisen condensation converts the fatty acid methyl ester 1 to a corresponding ketoester 2.

The conversion of the ketoester 2 resulting from Claisen condensation to a corresponding ketone 3 provides a ketone for the synthesis of a wide variety of lipids, including known structures as well as new classes of lipids. The ketoester 2 can be subjected to conditions effective to convert the ketoester 2 to a ketone 3 via the hydrolysis and/or decarboxylation. In FIG. 1, the conversion of ketoester 2 to ketone 3 is carried out with aqueous NaOH with subsequent addition of HCl and heat, although other suitable conditions can be selected by those of skill in the art.

Without being limiting, FIG. 1 shows that six classes of lipids can be made from the ketone 3. For example, the ketone 3 may serve as a substrate to produce various KC2 analogues. In particular, FIG. 1 shows ketone 3 serving as a substrate to make a nor-KC2 lipid (Scheme A), a sulfur KC2 analogue (Scheme H) or a KC2 branched sulfur lipid (Scheme I). Further, the ketone 3 can serve as a substrate to make a linoleate series of lipids (Scheme E), a saturated series of lipids (Scheme F) or a new series of lipids referred to herein as NVT1000 (Scheme J).

Yet further, as shown in FIG. 1, the ketone 3, can be converted to its corresponding alcohol 4 by a reduction reaction. Without intending to be limiting, the ketone 3 can be converted to alcohol 4 by treatment with sodium borohydride (NaBH₄) in an appropriate solvent. Such a reaction step is known to those of ordinary skill in the art and thus can be carried out using known techniques.

As shown in FIG. 1, the alcohol 4 may serve as a substrate to produce a number of different lipids or lipid classes. In particular, FIG. 1 shows alcohol 4 serving as a substrate to make a nor-MC3 lipid (Scheme B), a class of anionic carboxylate lipids (Scheme C), a class of dendripids (Scheme D), a class of sulfur lipids having substituted S atoms in their alkyl groups (Scheme G) or a class of lipids having three alkyls (R, R, R′) such as trialkyl lipids.

The following provides a more detailed description of synthetic routes A-K (FIG. 1). It will be appreciated by those of skill in the art that the synthetic routes set forth below are merely exemplary and additional or modified synthesis routes could readily be envisioned by those of skill in the art to make ionizable lipids.

### Schemes A and B: Synthesis Routes for Nor-KC2 and Nor-MC3 Lipids

The method described herein using Claisen condensation allows for the generation of new ionizable lipids, referred to herein as nor-MC3 and nor-KC2.

As discussed, the MC3 lipid, ((6Z,9Z,28Z,31Z)-heptatriacont-6,9,28,31-tetraene-19-yl 4-(dimethylamino)butanoate; structure below) is widely used in nucleic acid formulations, such as siRNA lipid nanoparticle (LNP) formulations. MC3 is widely regarded as a state-of-the art lipid in terms of its efficacy. Indeed, MC3 has been formulated in clinical formulations, including Onpattro®. MC3 is an improved version of KC2 (structure also below) and has been found to be about three times more efficacious. This means that formulations incorporating MC3 require about 3 times less siRNA to attain the same end result as similar formulations based on KC2. Nonetheless, the KC2 lipid remains a valuable research tool. The structures of MC3 and KC2 lipids are shown below:

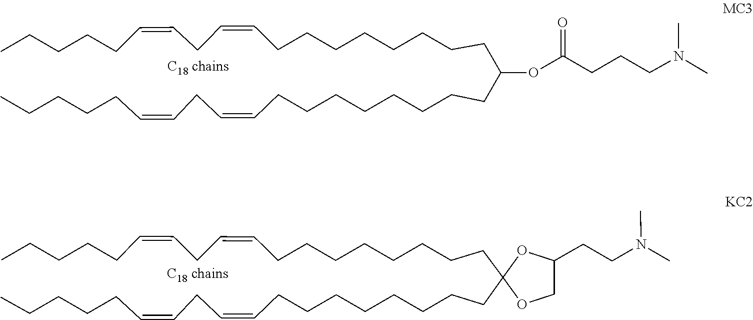

Both lipids have ionizable dimethylamino head groups. These lipids have two alkyl groups (R) converging into a single carbon atom, which in turn serves as the anchoring point for the ionizable head groups. Both MC3 and KC2 have alkyl groups that are Cis moieties derived from linoleic acid or a corresponding ester.

The conventional synthesis of MC3 requires six steps from methyl linoleate. This synthesis is shown in comparative Example 1 below. As discussed, this includes the preparation of an “MC3 alcohol” by a multiple-step synthesis including a lithium aluminum hydride (LiAlH₄ abbreviated “LAH”) reduction of methyl linoleate and a Grignard reaction. Steps that require LAH and Grignard reagents are best avoided due to their known fire hazard. The synthesis of KC2 also involves the preparation of an MC3 alcohol, but is followed by four additional steps to make the KC2 lipid, including an oxidation reaction that converts the MC3 alcohol to “KC2 ketone” that requires PCC: a reagent containing carcinogenic hexavalent chromium. Overall, a total of nine steps are required for the synthesis of KC2 using current methods.

The synthesis of nor-KC2 and nor-MC3 lipid derivatives using the disclosed method addresses these shortcomings and is described below.

### Scheme A: Synthesis of Nor-KC2

Methyl linoleate 5 is used as the starting material and is subjected to a mild Mukaiyama variant of the Claisen condensation (step 1 below) that is carried out at 0 degrees Celsius to room temperature and uses triethylamine as the base for the conversion reaction. This produces the ketoester 6, which is subjected to a hydrolysis and decarboxylation reaction to produce a corresponding ketone 7 as shown below:

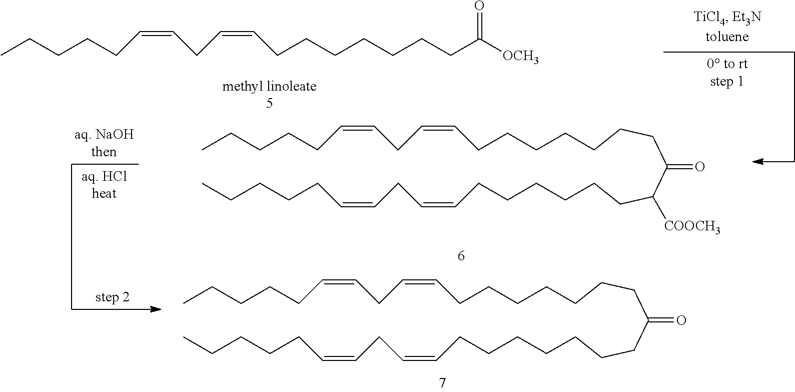 [SMILES: CCCCC/C=C\C/C=C\CCCCCCCC(=O)OC ; CCCCC/C=C\C/C=C\CCCCCCCC(=O)C(CCCCCC/C=C\C/C=C\CCCCC)C(=O)OC ; CCCCC/C=C\C/C=C\CCCCCCCC(=O)CCCCCCC/C=C\C/C=C\CCCCC]

Ketone 7 is converted into nor-KC2 in three steps by the following sequence (as shown previously):

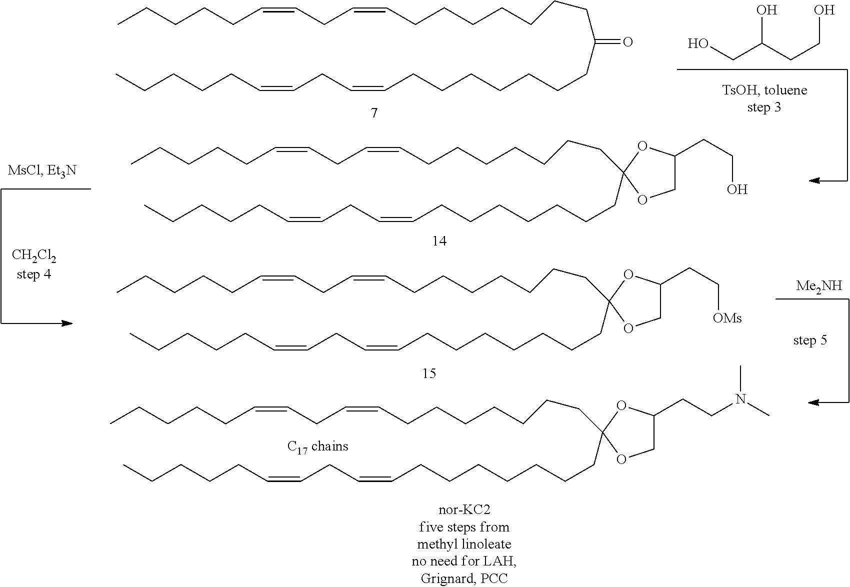 [SMILES: "Me2NH" ; CCCCC/C=C\C/C=C\CCCCCCCC(=O)CCCCCCC/C=C\C/C=C\CCCCC ; OCCC(O)CO ; CCCCC/C=C\C/C=C\CCCCCCCC1(CCCCCCC/C=C\C/C=C\CCCCC)OCC(CCO)O1 ; CCCCC/C=C\C/C=C\CCCCCCCC1(CCCCCCC/C=C\C/C=C\CCCCC)OCC(CCOS(C)(=O)=O)O1 ; CCCCC/C=C\C/C=C\CCCCCCCC1(CCCCCCC/C=C\C/C=C\CCCCC)OCC(CCN(C)C)O1]

Alternatively, ketone 7 is converted into nor-KC2 in one step in a synthesis comprising ketalization with an aminodiol hydrochloride such as 16:

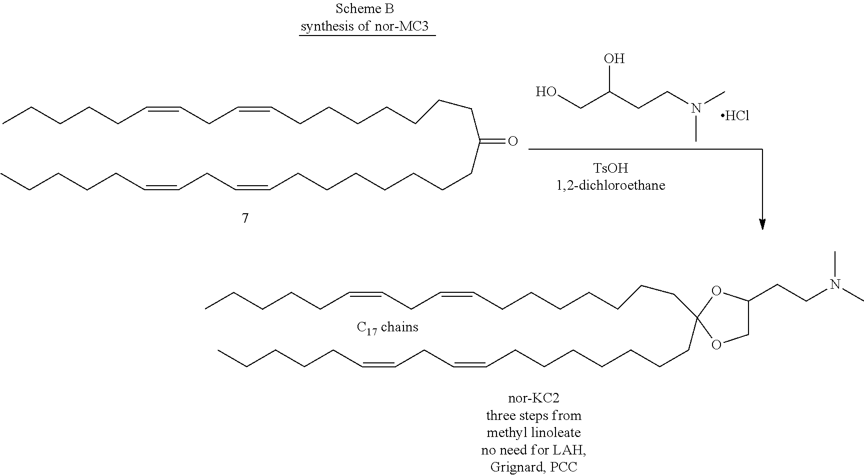

Methyl linoleate 5 is used as the starting material and is subjected to a mild Mukaiyama variant of the Claisen condensation (step 1) that may be carried out, for example, at 0 degrees Celsius to room temperature and may use triethylamine as the base for the conversion reaction. This produces the ketoester 6, which is subjected to a hydrolysis and/or decarboxylation of the ketoester to produce a corresponding ketone 7 as shown below:

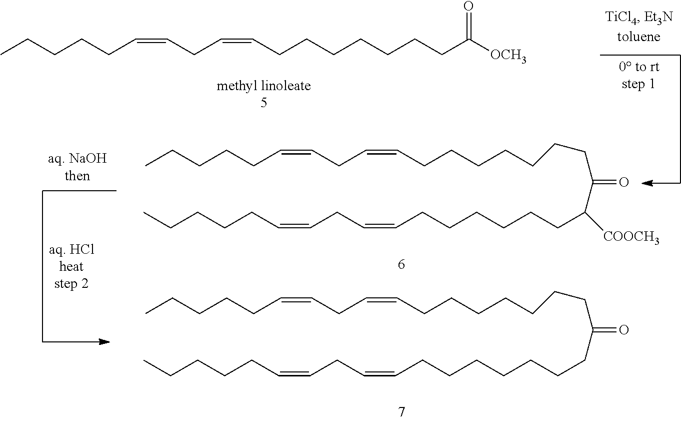 [SMILES: CCCCC/C=C\C/C=C\CCCCCCCC(=O)OC ; CCCCC/C=C\C/C=C\CCCCCCCC(=O)C(CCCCCC/C=C\C/C=C\CCCCC)C(=O)OC ; CCCCC/C=C\C/C=C\CCCCCCCC(=O)CCCCCCC/C=C\C/C=C\CCCCC]

The synthesis of nor-MC3 involves two steps from ketone 7 as shown below:

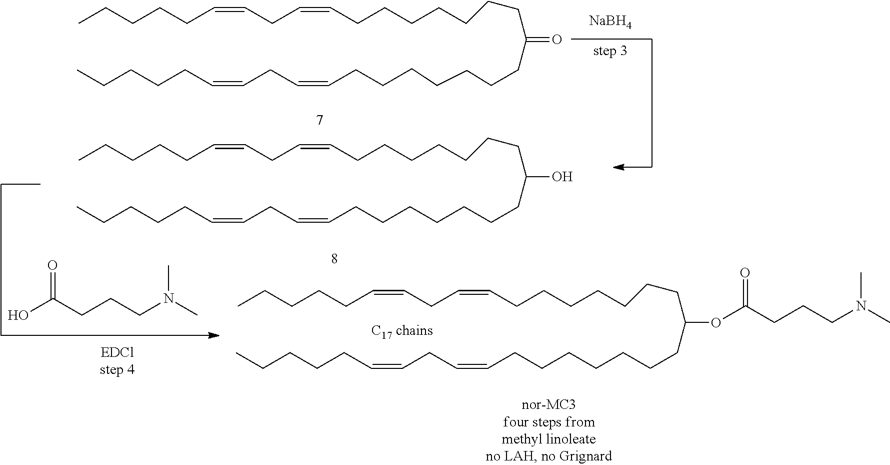

It will be evident from the above synthesis Schemes A and B that the new routes to nor-KC2 and nor-MC3 have fewer steps than those leading to the original KC2 (3 or 5 vs. 9 steps) and MC3 (4 vs. 6 steps) and additionally bypass the need for LAH, Grignard, and PCC use.

### Scheme C: Synthesis of Anionic Carboxylate Lipids

The lipid alcohol 22 can also be used to make anionic ionizable lipids, such as lipids having a head group with a terminal carboxylic acid (or carboxylate depending on the pH). Examples of such lipids include a lipid referred to herein as NVT604, 23, and in certain embodiments could be used to replace PEG-lipid conjugates. The structure of NVT604 is shown below:

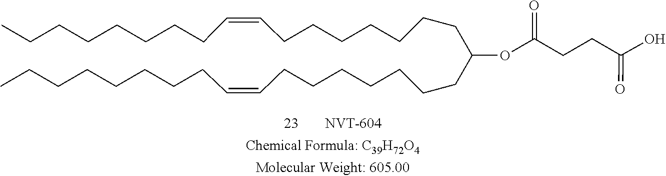 [SMILES: CCCCCCCC/C=C\CCCCCCCC(CCCCCCC/C=C\CCCCCCCC)OC(=O)CCC(=O)O]

The production of anionic lipids in this example employs methyl oleate similar to Scheme C, although those skilled in the art can readily envision the use of other suitable fatty acid methyl esters. Further, lipids could be produced from alternative head groups besides those derived from succinic acid as described below.

Claisen condensation of methyl oleate, 28, and the conversion of the resulting ketoester 29 to ketone 30 is depicted below. Reduction of the latter to alcohol 22 enabled the subsequent preparation of two new lipid types.

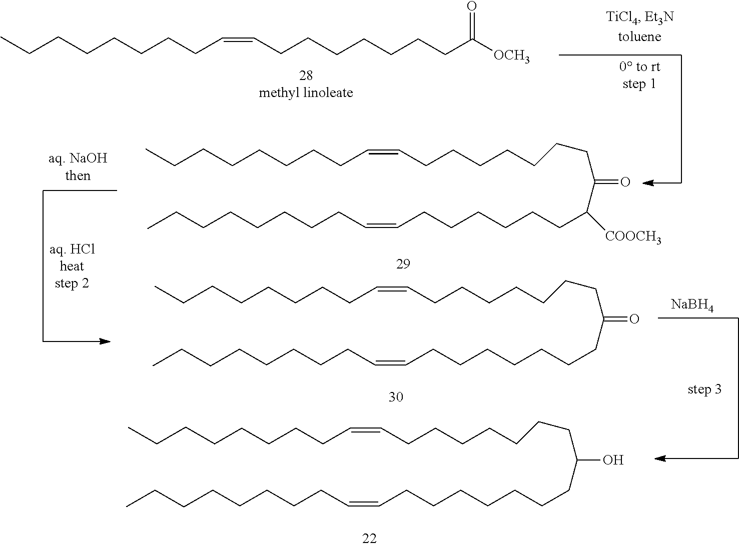

The reaction of 22 with succinic anhydride using known procedures produced NVT604, 23:

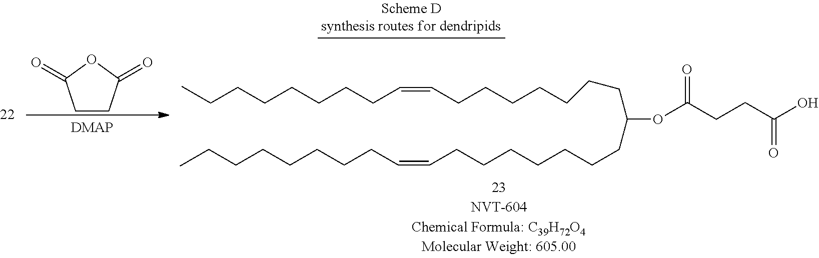

The alcohol 25 in synthesis Scheme C above can also be used to produce a new class of lipids referred to herein as “dendripids” with branched chains. Similar to MC3, the lipids comprise two alkyl groups converging into a single carbon atom, which in turn serves as an anchoring point for an ionizable head group. The two alkyl groups similarly each contain a carbon atom as a branch point that anchors two alkyl chains, each with a sulfur atom adjacent to the carbon branch point (see structure below).

A non-limiting example of a dendripid produced by Scheme D is compound 26 shown below:

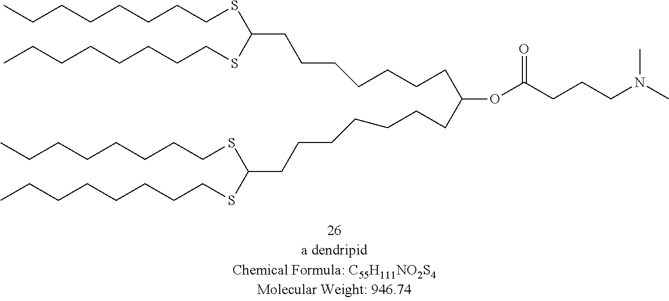 [SMILES: CCCCCCCCSC(CCCCCCCC(CCCCCCCC(SCCCCCCCC)SCCCCCCCC)OC(=O)CCCN(C)C)SCCCCCCCC]

The production of dendripids in this example employs methyl oleate (similar to Scheme C), although those skilled in the art could readily envision the use of other suitable fatty acid methyl esters as substrates as a starting material.

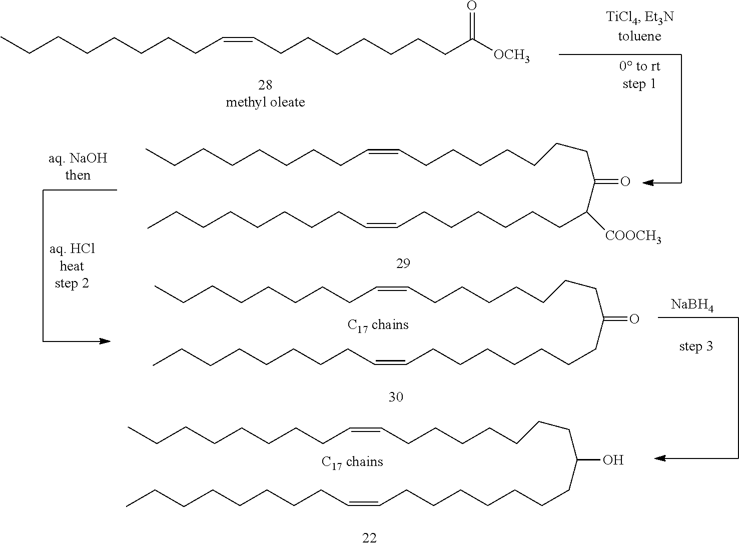

Alcohol 22 above can be used as a precursor to prepare new classes of dendripids by the sequence of reaction steps shown below. Dendripids may be particularly efficacious for the formulation and in vivo delivery of mRNA and other large nucleic acids.

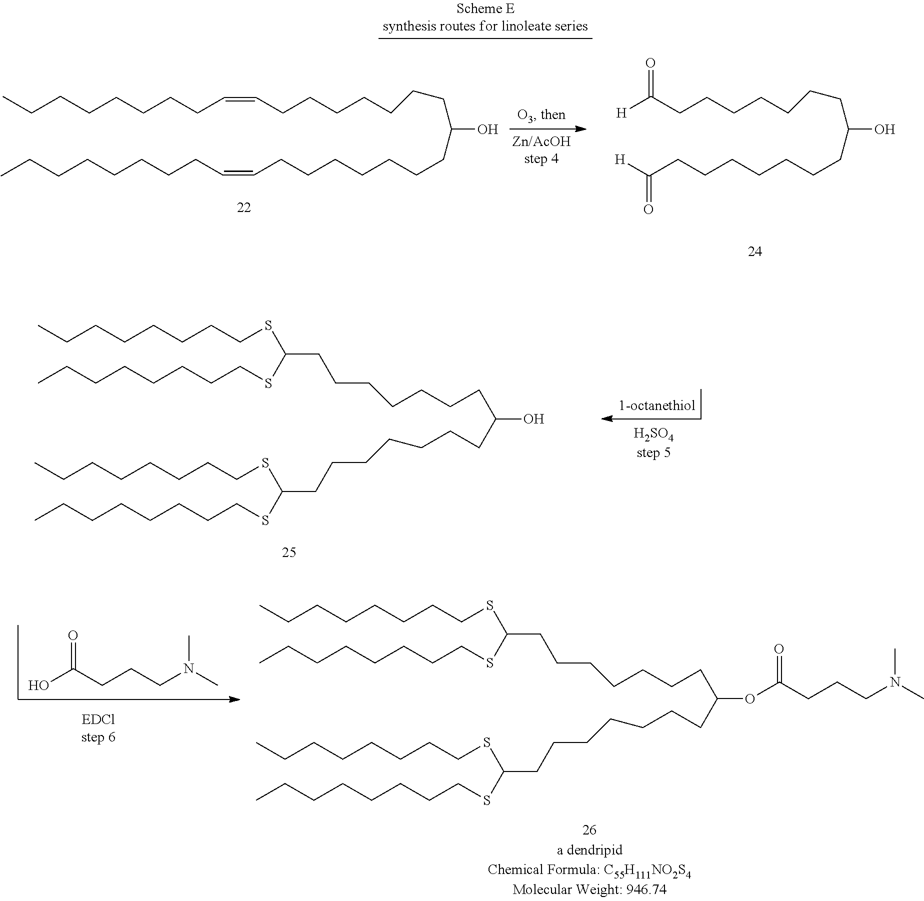

A linoleate series of lipids can be prepared using Claisen condensation using methyl linolenate, 31, as the starting fatty acid methyl ester.

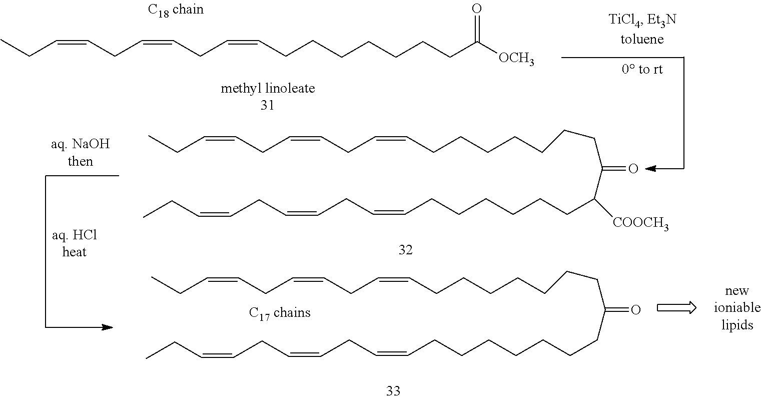 [SMILES: CC/C=C\C/C=C\C/C=C\CCCCCCCC(=O)OC ; CC/C=C\C/C=C\C/C=C\CCCCCCCC(=O)C(CCCCCC/C=C\C/C=C\C/C=C\CC)C(=O)OC ; CC/C=C\C/C=C\C/C=C\CCCCCCCC(=O)CCCCCCC/C=C\C/C=C\C/C=C\CC]

The fatty acid methyl ester 31 is converted to the ketoester 32 by Claisen condensation. The ketoester 32 is converted to ketone 33 by hydrolysis and decarboxylation as described.

The ketone 33 can be used as a precursor to prepare new ionizable lipids such as compounds 34 and 35, which are available by the same synthetic steps shown earlier for nor-MC3 and nor-KC2.

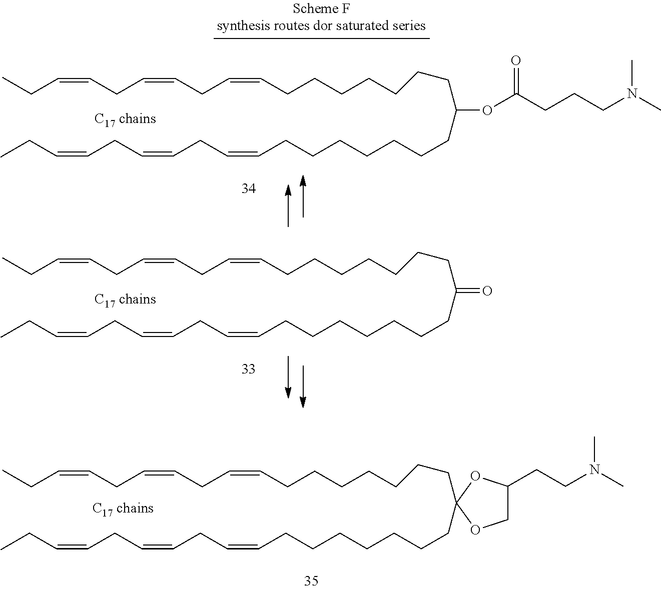

A saturated series of lipids can be prepared using Claisen condensation using saturated fatty acid methyl esters 36 as the starting material. Non-limiting examples of saturated methyl esters 36 include methyl myristate, methyl palmitate and methyl stearate. Claisen condensation produces the corresponding ketoester 37 from the fatty acid methyl ester 36. Hydrolysis and decarboxylation results in the ketone 38 that in turn serves as a precursor to prepare lipids.

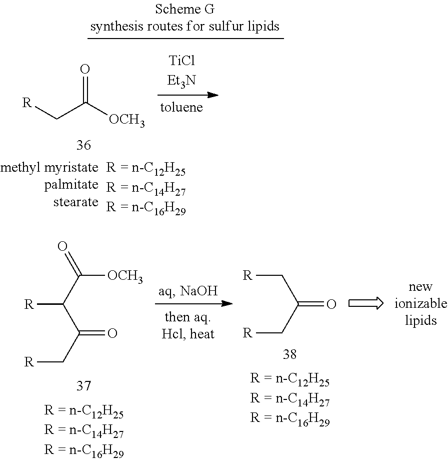

Sulfur-containing lipids may be produced from a fatty acid methyl ester that has one or more sulfur atoms in its alkyl group. An example of such a substrate for the Claisen condensation is shown below as fatty acid methyl ester 42. The fatty acid methyl ester 42 subjected to the Claisen condensation in this example is prepared from methyl oleate or olive oil that are subjected to a synthetic route involving ozonolysis of the double bond, with subsequent NaBH₄ reduction. This affords a hydroxyester such as 39, which can be converted to the fatty acid methyl ester 42 having alkyl chains di-substituted with sulfur. This fatty acid methyl ester 43 is then subjected to Claisen condensation to make the ketoester 2 as detailed below.

The preliminary ozonolysis and reduction with NaBH₄ to make the fatty acid methyl ester 42 comprising an alkyl group di-substituted with sulfur atoms is shown below:

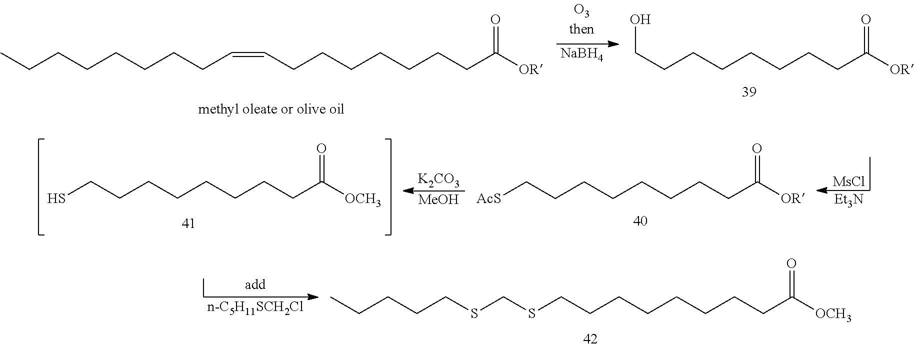

The steps of the synthesis scheme (subsequent to preliminary ozonolysis, NaBH₄ reduction of peroxidic intermediates, etc.) employing Claisen chemistry to produce the ketoester 43 from the S substituted fatty acid methyl ester 42 and the corresponding ketone 17 and alcohol 27 are shown below:

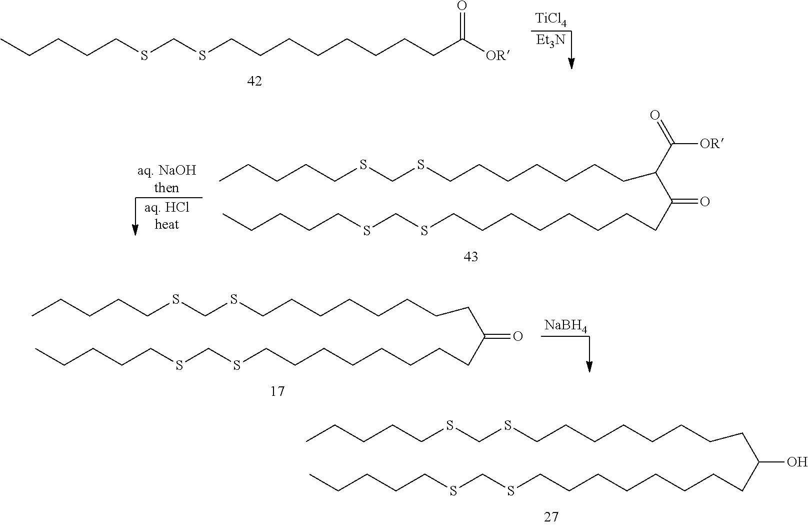

The alcohol 27 is subsequently used as a precursor to make a lipid referred to herein as MF19. In this example, coupling with 4-dimethylaminobutyric acid produces the MF19 ionizable lipid shown below.

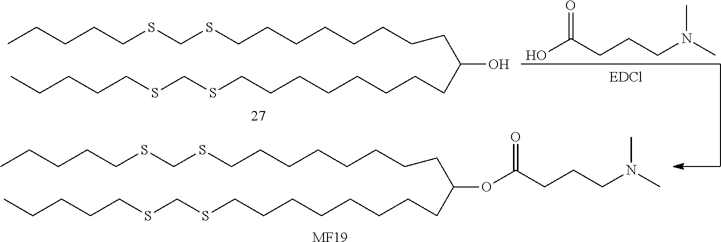

In addition to avoiding some protection/deprotection steps and bypassing the need for a Grignard reaction, the Claisen route provides ketone 17, which would be difficult to make by modifications using other methods to prepare MF19 ionizable lipids.

### Scheme H: Synthesis Routes for Sulfur KC2 Analogues

The above ketone 17 having alkyl groups di-substituted with S can be converted into ionizable lipid 20, which is a sulfur-containing analogue of KC2, thus enabling the production of yet another family of lipids. The synthetic route starting from ketone 17 (from Scheme G) is set forth below:

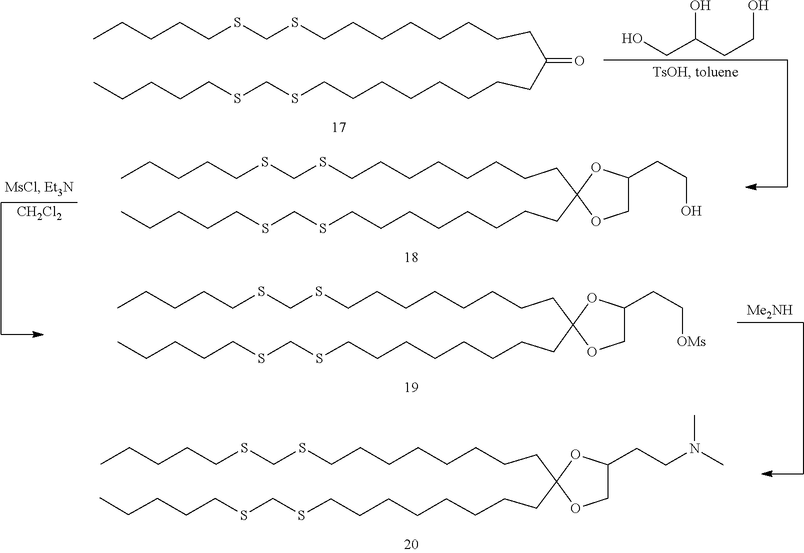

Alternatively, ketone 17 can be converted to compound 20 in a synthesis comprising ketalization with an aminodiol hydrochloride such as 16:

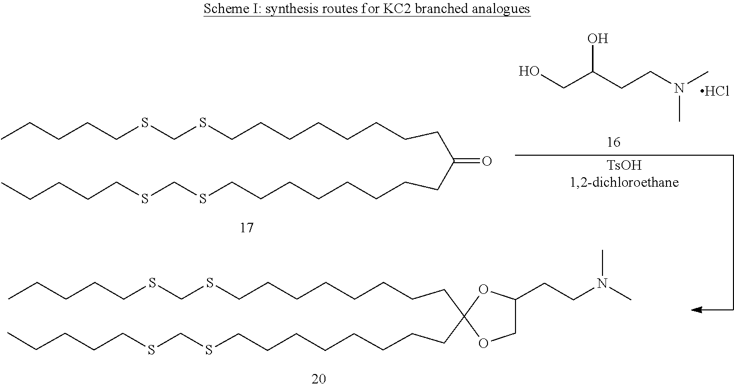

A similar strategy to that of Scheme H above may be implemented to produce dendripid ketone 13, which can be converted into a KC2-like branched sulfur lipid xx (see end product of scheme below). While oxidation of a dendripid alcohol 24 (see scheme D above) would also yield 13, the sulfur atoms in the substrate render this transformation challenging, underscoring the value of the Claisen approach of the present disclosure.

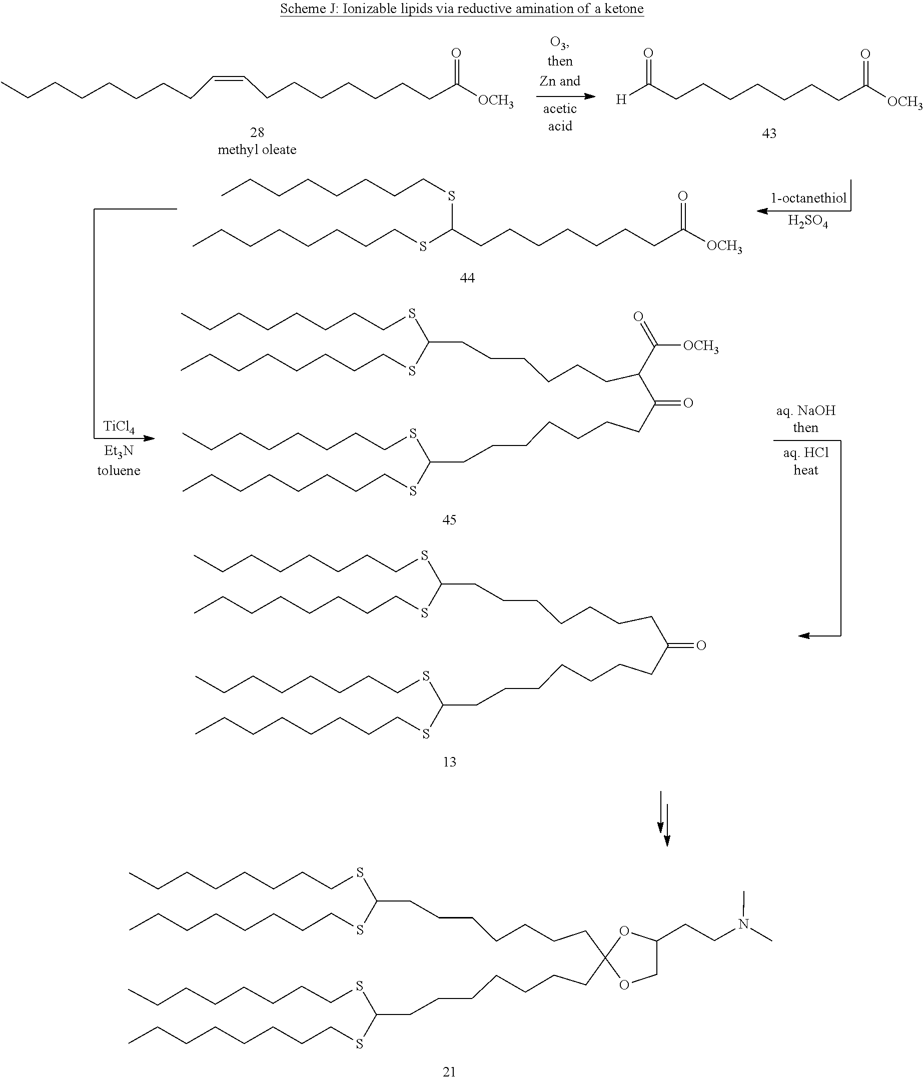

The ketones 3 described above may be used as precursors to prepare ionizable lipids via reductive amination with an aminoalcohol or a an O-protected form thereof. This is exemplified in the scheme below with the conversion of ketone 7 into ionizable lipid 48.

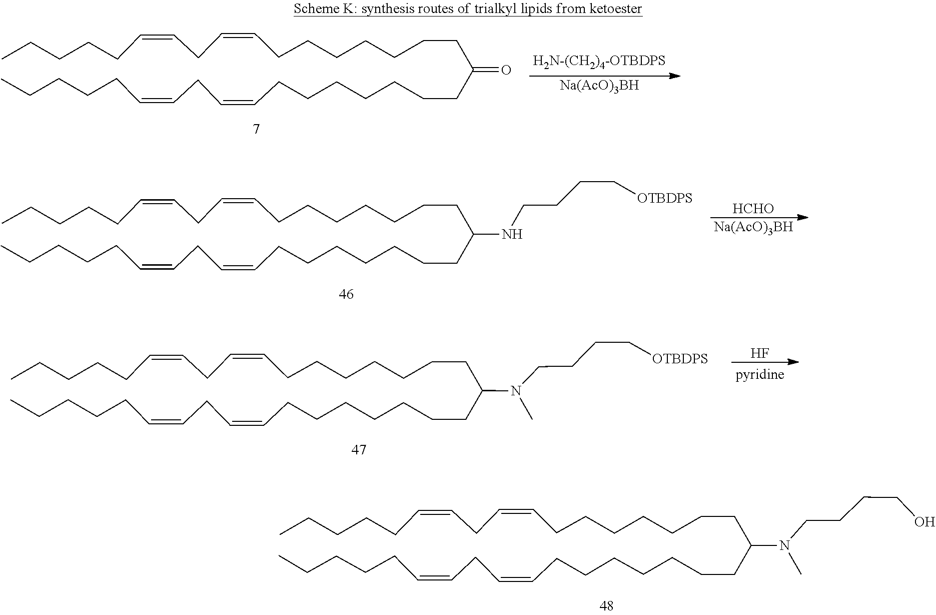

Likewise, the ketoesters 2 produced by Claisen condensation shown earlier, and represented below as structure 2, can be used to prepare branched analogues of KC2 (compound 49) and MC3 (50):

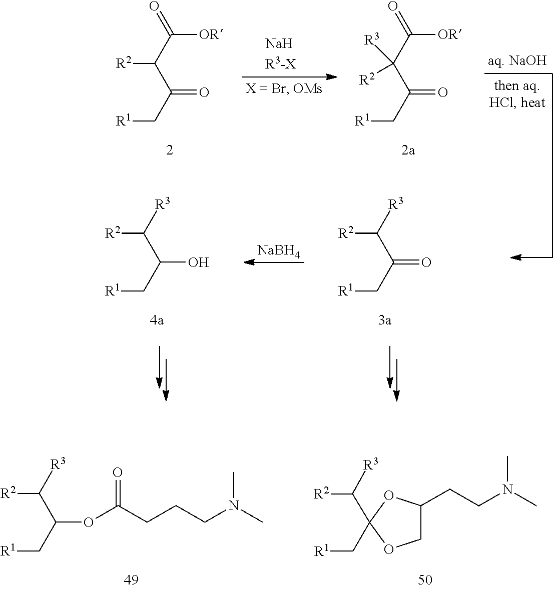

The ketoester 2 is reacted with R³−X (X=halogen such as Cl, Br, I, or sulfonate such as MsO, TsO, and the like) in this example to prepare a ketoester 2a. The ketoester 2a having three alkyl groups is subjected to hydrolysis and decarboxylation to produce ketone 3a. The ketone may be used to prepare branched analogues of KC2, depicted by structure 46, or it may be reduced with NaBH₄ in an appropriate solvent to make the alcohol 4a. The alcohol 4a is used as an intermediate to prepare branched analogues of MC3 as depicted by structure 47.

In addition, ketones 3a having three alkyl groups can be used as precursors to prepare branched analogues of lipids of the type 48 described in Scheme J above. Synthesis of branched analogues of lipids of the type 48 from a ketone is set forth below:

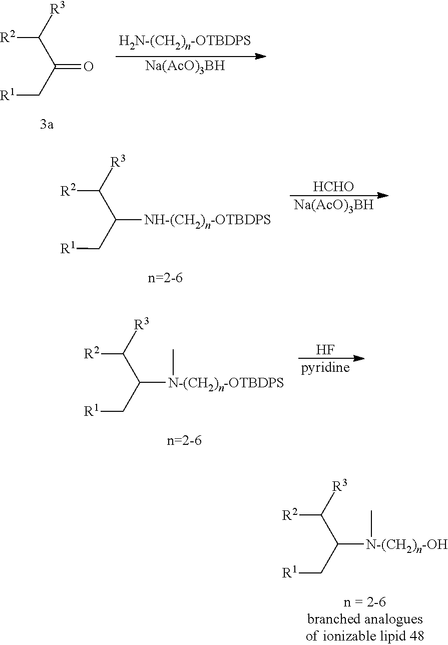

### Lipids Produced by the Foregoing Synthesis Schemes

The ionizable lipids produced by the method disclosed herein may include new ionizable lipids. Alternatively, the method may be used to synthesize known lipids. In one embodiment, the ionizable lipid is a lipid having an ionizable amino, carboxylic acid and/or hydroxyl group.

In one embodiment, the ionizable lipids produced are selected from nor-KC2 and analogues thereof, nor-MC3 and analogues thereof, linoleate lipids, saturated lipids, NVT1000 lipids, anionic ionizable lipids, dendripids, sulfur lipids and trialkyl lipids.

The following provides non-limiting examples of ionizable lipid structures produced by Schemes A-K described above. It will be understood, however, that the structures below are merely exemplary of ionizable lipids that can be prepared from the method described herein and should not be considered limiting to the present disclosure.

### Formula A: Nor-KC2 and Analogues Thereof

Nor-KC2 and analogues thereof may be represented by Formula A:

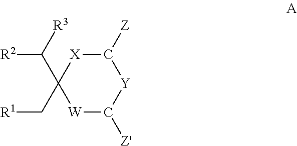

wherein each R¹ and R² group is, independently, a linear or branched alkyl group having from 4 to 30 carbon atoms, and wherein the alkyl groups may incorporate (i) from 0 to 4 heteroatoms, such as sulfur or oxygen atoms, (ii) from 0 to 5 C═C double bonds of E or Z geometry, and/or (iii) substituents such as OH, O-alkyl, S-alkyl, and N(alkyl)₂ bonded to a carbon atom, (iv) alkyl substituent having less than 5 carbon atoms, such as linear or branched substituents, including moieties selected from methyl, ethyl, propyl, isopropyl, butyl, isobutyl, and tert-butyl. R³ may be H or a linear or branched alkyl group having from 4 to 30 carbon atoms, that may incorporate (i) from 0 to 4 heteroatoms, such as sulfur or oxygen atoms, (ii) from 0 to 5 C═C double bonds of E or Z geometry, and/or (iii) substituents such as OH, O-alkyl, S-alkyl, and N(alkyl)₂ bonded to a carbon atom, (iv) alkyl substituent having less than 5 carbon atoms, such as linear or branched substituents, including moieties selected from methyl, ethyl, propyl, isopropyl, butyl, isobutyl, and tert-butyl.

W and X are each, independently O or S;

Y is absent (the two C's are directly connected), or if Y is present is selected from: (i) a metheno (C₁) bridge optionally substituted with a short alkylamino group of the type [(CH₂)ₙ-NG¹G²], wherein n=1-5 and G¹ and G² are, independently, a small alkyl having less than 5 carbon atoms (e.g., methyl, ethyl, propyl, isopropyl, butyl, isobutyl, and tert-butyl), or portions of a 4-7-membered ring containing N, so that NG¹G² is a nitrogen heterocycle moiety such as a 1-azetidinyl, 1-pyrrolidinyl, 1-piperidinyl, 1-azepanyl, 1-morpholinyl, 1-thiomorpholinyl, 1-piperazinyl; or
(ii) an etheno (C₂) bridge optionally substituted with a short alkylamino group as specified above for the metheno case;
Z and Z′ are, independently, H, or a short alkylamino group as stated above for the metheno case.

In one embodiment, the lipid of Formula A is the nor-KC2 lipid described herein.

### Formula B: Nor-MC3 and Analogues Thereof

Lipids that are analogues or MC3 may be represented with Formula B, wherein:

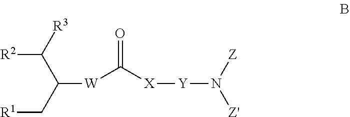

wherein each R¹ and R² group is, independently, a linear or branched alkyl group having from 4 to 30 carbon atoms, and wherein the alkyl groups may incorporate (i) from 0 to 4 heteroatoms, such as sulfur or oxygen atoms, (ii) from 0 to 5 C═C double bonds of E or Z geometry, and/or (iii) substituents such as OH, O-alkyl, S-alkyl, and N(alkyl)₂ bonded to a carbon atom, (iv) alkyl substituent having less than 5 carbon atoms, such as linear or branched substituents, including moieties selected from methyl, ethyl, propyl, isopropyl, butyl, isobutyl, and tert-butyl. R³ may be H or a linear or branched alkyl group having from 4 to 30 carbon atoms, that may incorporate (i) from 0 to 4 heteroatoms, such as sulfur or oxygen atoms, (ii) from 0 to 5 C═C double bonds of E or Z geometry, and/or (iii) substituents such as OH, O-alkyl, S-alkyl, and N(alkyl)₂ bonded to a carbon atom, (iv) alkyl substituent having less than 5 carbon atoms, such as linear or branched substituents, including moieties selected from methyl, ethyl, propyl, isopropyl, butyl, isobutyl, and tert-butyl.

W is NH, or
N-small alkyl, such as N—CH₃, or
O

X is NH, or
N-small alkyl such as N—CH₃, or
O, or
CG¹G², wherein G¹ and G² are, independently, H or the short-chain alkyl substituent;

Y is a short linear chain of 1-5 carbon atoms, and optionally substituted at one or more positions with the short-chain alkyl substituent;

Z and Z′ are independently the short-chain alkyl substituent, or portions of a 4-7-membered ring containing N, so that NZZ′ is a nitrogen heterocycle residue such as a 1-azetidinyl, 1-pyrrolidinyl, 1-piperidinyl, 1-azepanyl, 1-morpholinyl, 1-thiomorpholinyl, 1-piperazinyl.

### Formula C: Anionic Ionizable Lipids and Analogues Thereof

Anionic ionizable lipids produced by the schemes above may be represented by Formula C having the following structure:

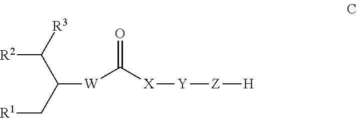

wherein each R¹ and R² group is, independently, a linear or branched alkyl group having from 4 to 30 carbon atoms, and wherein the alkyl groups may incorporate (i) from 0 to 4 heteroatoms, such as sulfur or oxygen atoms, (ii) from 0 to 5 C═C double bonds of E or Z geometry, and/or (iii) substituents such as OH, O-alkyl, S-alkyl, and N(alkyl)₂ bonded to a carbon atom, (iv) alkyl substituent having less than 5 carbon atoms, such as linear or branched substituents, including moieties selected from methyl, ethyl, propyl, isopropyl, butyl, isobutyl, and tert-butyl. R³ may be H or a linear or branched alkyl group having from 4 to 30 carbon atoms, that may incorporate (i) from 0 to 4 heteroatoms, such as sulfur or oxygen atoms, (ii) from 0 to 5 C═C double bonds of E or Z geometry, and/or (iii) substituents such as OH, O-alkyl, S-alkyl, and N(alkyl)₂ bonded to a carbon atom, (iv) alkyl substituent having less than 5 carbon atoms, such as linear or branched substituents, including moieties selected from methyl, ethyl, propyl, isopropyl, butyl, isobutyl, and tert-butyl.

W is NH, or
N-small alkyl such as N—CH₃, or
O;

X is NH, or
N-small alkyl such as N—CH₃, or
, or
CG¹G², wherein G¹ and G² are, independently, H or the short-chain alkyl substituent;
   Y is a Short Linear Chain of 1-5 Carbon Atoms, and Optionally Substituted at One or More Positions with the Short-Chain Alkyl Substituent;
   Z—H is an ionizable functionality capable of releasing the H as H+(=a proton) to produce an anion

Z−, and exhibiting a pKa comprised between 2 and 10. Examples of such ionizable functionalities are: —NHCOCOOH (pKa˜ 2), 1,3-dithiane-2-carboxylic acid (pKa˜ 3), —OCH₂COOH (pKa˜ 4), COOH and tetrazole (pKa˜ 5), 1,2,4-oxadiazolin-5-one (pKa˜6), -hydroxamic acid (pKa˜ 9), phenol and primary sulfonamide (pKa˜ 10).

### Formulas D and E: Dendripids

These compounds (exemplified below) may be represented with Formula D having the following structure:

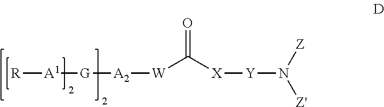

wherein each R is, independently, a linear or branched alkyl group having from 4 to 30 carbon atoms, and wherein the alkyl groups may incorporate (i) from 0 to 4 heteroatoms, such as sulfur or oxygen atoms, (ii) from 0 to 5 C═C double bonds of E or Z geometry, and/or (iii) substituents such as OH, O-alkyl, S-alkyl, and N(alkyl)₂ bonded to a carbon atom, (iv) alkyl substituent having less than 5 carbon atoms, such as linear or branched substituents, including moieties selected from methyl, ethyl, propyl, isopropyl, butyl, isobutyl, and tert-butyl.

A¹ is 0 or S

G is a linear alkyl group comprising between 2 and 18 carbon atoms, 0-4 double bonds that may be of Z geometry, the linear alkyl chain optionally substituted at one or more positions with a linear or branched short-chain alkyl substituent having less than 5 carbon atoms, such as linear or branched substituents, including moieties selected from methyl, ethyl, propyl, isopropyl, butyl, isobutyl, and tert-butyl;

A² is CH, or
the short-chain alkyl substituent, or
C—OH;

W is NH, or
N-small alkyl such as N—CH3, or
O;

X is NH, or
N-small alkyl such as N—CH₃, or
O, or
CG¹G², wherein G¹ and G² are, independently, H or the short chain alkyl,

Y is a short linear chain of 1-5 carbon atoms, and facultatively exhibiting one or more small alkyl groups (Me, Et . . . )

Z and Z′ are independently the short chain alkyl, or
portions of a 4-7-membered ring containing N, so that NZZ′ is a nitrogen heterocycle residue such as a 1-azetidinyl, 1-pyrrolidinyl, 1-piperidinyl, 1-azepanyl, 1-morpholinyl, 1-thiomorpholinyl, 1-piperazinyl.

KC2-type dendripids may be represented by Formula E, wherein:

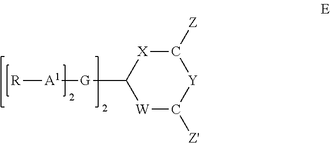

wherein each R is, independently, a linear or branched alkyl group having from 4 to 30 carbon atoms, and wherein the alkyl groups may incorporate (i) from 0 to 4 heteroatoms, such as sulfur or oxygen atoms, (ii) from 0 to 5 C═C double bonds of E or Z geometry, and/or (iii) substituents such as OH, O-alkyl, S-alkyl, and N(alkyl)₂ bonded to a carbon atom, (iv) alkyl substituent having less than 5 carbon atoms, such as linear or branched substituents, including moieties selected from methyl, ethyl, propyl, isopropyl, butyl, isobutyl, and tert-butyl.

### A¹ is 0 or S

G is a linear alkyl chain comprising between 2 and 18 carbon atoms, 0-4 double bonds that may be of Z geometry, the linear alkyl chain optionally substituted at one or more positions with a linear or branched short-chain alkyl substituent having less than 5 carbon atoms, such as linear or branched substituents, including moieties selected from methyl, ethyl, propyl, isopropyl, butyl, isobutyl, and tert-butyl;
W and X are each, independently O or S;
Y is absent (the two C's are directly connected), or if Y is present is selected from: (i) a metheno (C₁) bridge optionally substituted with a short alkylamino group of the type [(CH₂)ₙ-NG¹G²], wherein n=1-5 and G¹ and G² are, independently, a small alkyl having less than 5 carbon atoms (e.g., methyl, ethyl, propyl, isopropyl, butyl, isobutyl, and tert-butyl), or portions of a 4-7-membered ring containing N, so that NG¹G² is a nitrogen heterocycle moiety such as a 1-azetidinyl, 1-pyrrolidinyl, 1-piperidinyl, 1-azepanyl, 1-morpholinyl, 1-thiomorpholinyl, 1-piperazinyl; or
(ii) an etheno (C₂) bridge optionally substituted with a short alkylamino group as specified above for the metheno case;
Z and Z′ are, independently, H, or a short alkylamino group as stated above for the metheno case.

### Formula F: Sulfur Lipids

Sulfur lipids produced by the method may be represented by Formula F having the structure below:

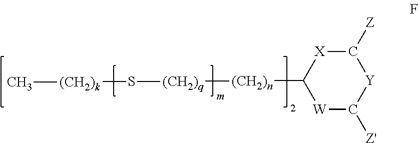

k may be 1-8,
m may be 1-8,
n may independently be 1 to 8,
q may independently be 1 to 8,
W and X are each, independently O or S;

Y is absent (the two C's are directly connected), or if Y is present is selected from:
(i) a metheno (C₁) bridge optionally substituted with a short alkylamino group of the type [(CH₂)ₙ-NG¹G²], wherein n=1-5 and G¹ and G² are, independently, a small alkyl having less than 5 carbon atoms (e.g., methyl, ethyl, propyl, isopropyl, butyl, isobutyl, and tert-butyl), or portions of a 4-7-membered ring containing N, so that NG¹G² is a nitrogen heterocycle moiety such as a 1-azetidinyl, 1-pyrrolidinyl, 1-piperidinyl, 1-azepanyl, 1-morpholinyl, 1-thiomorpholinyl, 1-piperazinyl; or
(ii) an etheno (C₂) bridge optionally substituted with a short alkylamino group as specified above for the metheno case;

Z and Z′ are, independently, H, or a short alkylamino group as stated above for the metheno case.

### Formula G: Lipids of the Type 48 and Branched Analogues Thereof

Ionizable lipids of the type 48 and branched analogues thereof may be represented with Formula G, wherein:

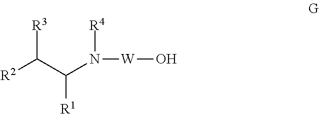

wherein R¹, R², and R³ are each, independently, a linear or branched alkyl group having from 4 to 30 carbon atoms, and wherein the alkyl groups may incorporate (i) from 0 to 4 heteroatoms, such as sulfur or oxygen atoms, (ii) from 0 to 5 C═C double bonds of E or Z geometry, and/or (iii) substituents such as OH, O-alkyl, S-alkyl, and N(alkyl)₂ bonded to a carbon atom, (iv) alkyl substituent having less than 5 carbon atoms, such as linear or branched substituents, including moieties selected from methyl, ethyl, propyl, isopropyl, butyl, isobutyl, and tert-butyl. R³ may be H or a linear or branched alkyl group having from 4 to 30 carbon atoms, that may incorporate (i) from 0 to 4 heteroatoms, such as sulfur or oxygen atoms, (ii) from 0 to 5 C═C double bonds of E or Z geometry, and/or (iii) substituents such as OH, O-alkyl, S-alkyl, and N(alkyl)₂ bonded to a carbon atom, (iv) alkyl substituent having less than 5 carbon atoms, such as linear or branched substituents, including moieties selected from methyl, ethyl, propyl, isopropyl, butyl, isobutyl, and tert-butyl.

R⁴ is a C₁-C₄ alkyl group

W is an alkyl chain containing between 2 and 6 carbon atoms, arranged in a linear or cyclic fashion.

### Formulation of the Ionizable Lipid in a Delivery Vehicle

The ionizable lipid produced by the method of the disclosure may be formulated in a variety of delivery vehicles known to those of ordinary skill in the art. An example of a delivery vehicle is a lipid nanoparticle, which includes liposomes, lipoplexes, polymer nanoparticles comprising lipids, polymer-based nanoparticles, emulsions, and micelles.

In one embodiment, the ionizable lipids are formulated in a delivery vehicle by mixing them with additional lipids, including helper lipids, such as vesicle forming lipids and optionally an aggregation inhibiting lipid, such as a hydrophilic polymer-lipid conjugate (e.g., PEG-lipid).

As set forth previously, a helper lipid includes a sterol, a diacylglycerol, a ceramide or derivatives thereof.

Examples of sterols include cholesterol, or a cholesterol derivative, such as cholestanol, cholestanone, cholestenone, coprostanol, cholesteryl-2′-hydroxyethyl ether, cholesteryl-4′-hydroxybutyl ether, beta-sitosterol, fucosterol, and the like.

Examples of diacylglycerols include dipalmitoylphosphatidylcholine (DPPC), distearoylphosphatidylcholine (DSPC), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), egg phosphatidylcholine (EPC), and mixtures thereof. In certain embodiments, the phospholipid is DPPC, DSPC, or mixtures thereof. These lipids may be synthesized or obtained from natural sources, such as from egg.

A suitable ceramide derivative is egg sphingomyelin.

Delivery vehicles incorporating the ionizable lipids can be prepared using a wide variety of well described formulation methodologies known to those of skill in the art, including but not limited to extrusion, ethanol injection and in-line mixing. Such methods are described in Maclachlan, I. and P. Cullis, “Diffusible-PEG-lipid Stabilized Plasmid Lipid Particles”, Adv. Genet., 2005. 53PA:157-188; Jeffs, L. B., et al., “A Scalable, Extrusion-free Method for Efficient Liposomal Encapsulation of Plasmid DNA”, Pharm Res, 2005. 22(3):362-72; and Leung, A. K., et al., “Lipid Nanoparticles Containing siRNA Synthesized by Microfluidic Mixing Exhibit an Electron-Dense Nanostructured Core”, The Journal of Physical Chemistry. C, Nanomaterials and Interfaces, 2012, 116(34): 18440-18450, each of which is incorporated herein by reference in its entirety.

The delivery vehicle can also be a nanoparticle that is a lipoplex that comprises a lipid core stabilized by a surfactant. Vesicle-forming lipids may be utilized as stabilizers. The lipid nanoparticle in another embodiment is a polymer-lipid hybrid system that comprises a polymer nanoparticle core surrounded by stabilizing lipid.

Nanoparticles comprising the ionizable lipid may alternatively be prepared from polymers without lipids. Such nanoparticles may comprise a concentrated core of a therapeutic agent that is surrounded by a polymeric shell or may have a solid or a liquid dispersed throughout a polymer matrix.

The ionizable lipids described herein can also be incorporated into emulsions, which are drug delivery vehicles that contain oil droplets or an oil core. An emulsion can be lipid-stabilized. For example, an emulsion may comprise an oil filled core stabilized by an emulsifying component such as a monolayer or bilayer of lipids.

The ionizable lipid may be incorporated into a micelle. Micelles are self-assembling particles composed of amphipathic lipids or polymeric components that are utilized for the delivery of agents present in the hydrophobic core.

A further class of drug delivery vehicles known to those of skill in the art that can be used to formulate the ionizable lipid herein is a carbon nanotube.

### Delivery of Nucleic Acid, Genetic Material, Proteins, Peptides or Other Charged Agents

The ionizable lipid disclosed herein may facilitate the incorporation of a compound or molecule (referred to herein also as “cargo” or “cargo molecule”) bearing a net negative or positive charge into the delivery vehicle and subsequent delivery to a target cell in vitro or in vivo.

In one embodiment, the molecule is genetic material, such as a nucleic acid. The nucleic acid includes, without limitation, RNA, including small interfering RNA (siRNA), small nuclear RNA (snRNA), micro RNA (miRNA), messenger RNA (mRNA) or DNA such as plasmid DNA or linear DNA. The nucleic acid length can vary and can include nucleic acid of 5-50,000 nucleotides in length. The nucleic acid can be in any form, including single stranded DNA or RNA, double stranded DNA or RNA, or hybrids thereof. Single stranded nucleic acid includes antisense oligonucleotides.

In one particularly advantageous embodiment, the cargo is an siRNA. An siRNA becomes incorporated into endogenous cellular machineries to result in mRNA breakdown, thereby preventing transcription. Since RNA is easily degraded, its incorporation into a delivery vehicle can reduce or prevent such degradation, thereby facilitating delivery to a target site.

Gene editing systems can also be incorporated into delivery vehicles comprising the charged lipid. This includes a Cas9-CRISPR, TALEN and zinc finger nuclease gene editing system. In the case of Cas9-CRISPR, a guide RNA (gRNA), together with a plasmid or mRNA encoding the Cas9 protein may be incorporated into a delivery vehicle comprising the ionizable lipid described herein. Optionally, a ribonucleoprotein complex may be incorporated into a delivery vehicle comprising the ionizable lipid described herein. Likewise, the disclosure includes embodiments in which genetic material encoding DNA binding and cleavage domains of a zinc finger nuclease or TALEN system are incorporated into a delivery vehicle together with the ionizable lipid.

The ionizable lipid may also facilitate the incorporation of proteins and peptides into a delivery vehicle, which includes ribonucleoproteins. This includes both linear and non-linear peptides, proteins or ribonucleoproteins.

While pharmaceutical compositions are described above, the ionizable lipid can be a component of any nutritional, cosmetic, cleaning or foodstuff product.

The following examples are given for the purpose of illustration only and not by way of limitation on the scope of the invention.

### EXAMPLES

Example 1 is a comparative example and exemplifies certain advantages of the method of the present disclosure over a more conventional lipid synthesis scheme. The production of nor-KC2 and nor-MC3 lipids verses KC2 and MC3 traditional synthesis is provided as the illustrative example.

Examples 2-14 set forth in more detail experimental procedures for the novel synthesis reactions of the present disclosure to produce a broad range of new and useful ionizable lipids.

Reagents and experimental protocols: For the experimental procedures (Examples 2-14), unless otherwise specified, all reagents and solvents were commercial products and were used without further purification, except THF (freshly distilled from Na/benzophenone under Ar), CH₂Cl₂ (freshly distilled from CaH₂ under Ar). “Dry methanol” was freshly distilled from magnesium turnings. All reactions were performed under an argon atmosphere. Reaction mixture from aqueous workups were dried by passing over a plug of anhydrous Na₂SO₄ held in a filter tube and rotary-evaporated under reduced pressure. Thin-layer chromatography was performed on silica gel plates coated with silica gel (Merck 60 F254 plates) and column chromatography was performed on 230-400 mesh silica gel. Visualization of the developed chromatogram was performed by staining with I₂ or potassium permanganate solution. Nuclear magnetic resonance spectra, ¹H (300 MHz) and ¹³C NMR (75 MHz), were recorded at room temperature in CDCl₃ solutions. ¹H NMR spectra were referenced to residual CHCl₃ (7.26 ppm) and ¹³C NMR spectra were referenced to the central line of the CDCl₃ triplet (77.00 ppm). Chemical shifts are reported in parts per million (ppm) on the 6 scale. Multiplicities are reported as “s” (singlet), “d” (doublet), “t” (triplet), “q” (quartet), “m” (multiplet), and further qualified as “app” (apparent) and “br” (broad). Low- and high-resolution mass spectra (m/z) were obtained in the electrospray (ESI) and field desorption/field ionisation (FD/FI) mode.

### Comparative Example 1: Traditional Synthesis of KC2 and MC3 vs the Inventive Synthetic Scheme to Prepare Nor-KC2 and Nor-MC3

As discussed, the traditional synthesis of KC2 and MC3 lipids is lengthy and requires the use of chemicals that pose safety and disposal risks. The inventors investigated a new synthesis route that overcomes these problems and that yielded new derivatives of KC2 and MC3 (among other lipids), referred to herein as nor-KC2 and nor-MC3. The new synthesis route employs a mild version of a Claisen condensation to produce a ketone that can be used as a starting material to make nor-KC2 and nor-MC3. While the discussion below outlines the production of nor-KC2 and nor-MC3, the ketone can also be used as a starting material to synthesize a variety of new lipid classes (see e.g., Schemes A-K above).

### (a) Traditional Synthesis of MC3 and KC2 Lipids

The traditional synthesis of KC2 and MC3 lipids uses methyl linoleate as a starting material to prepare “MC3 alcohol” in five steps:

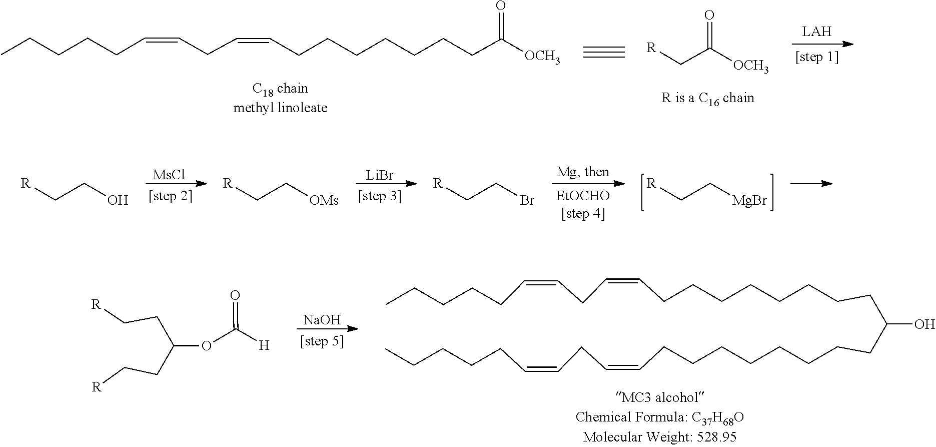

An additional step converts MC3 alcohol into actual MC3. The MC3 lipid is thus accessible from methyl linoleate in a total of 6 steps:

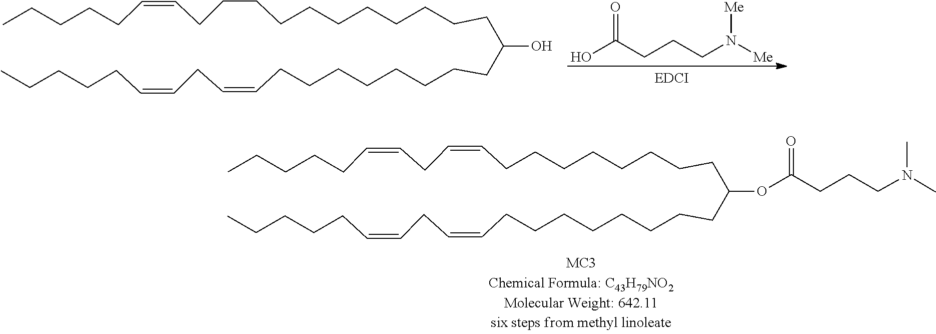 [SMILES: CCCCC/C=C\C/C=C\CCCCCCCCC(O)CCCCCCCCCCC/C=C\CCCCC ; CN(C)CCCC(=O)O ; CCCCC/C=C\C/C=C\CCCCCCCCC(CCCCCCCC/C=C\C/C=C\CCCCC)OC(=O)CCCN(C)C]

In contrast, the production of KC2 lipid from MC3 alcohol requires four additional steps. KC2 is thus available from methyl linoleate in a total of nine steps:

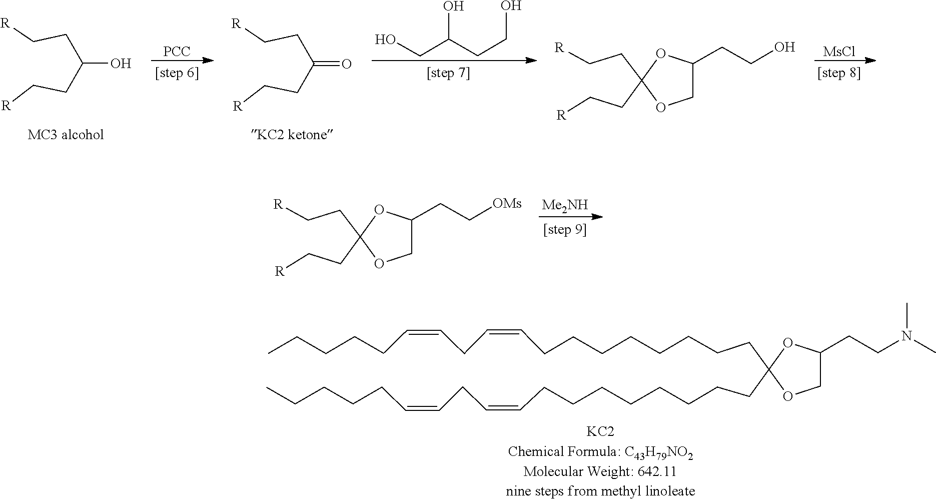

### (b) Drawbacks of the Traditional Synthesis Route

The inventors have recognized that a synthesis utilizing fewer steps than the traditional route outlined above and that also avoids hazardous reagents would be highly desirable. Such a synthesis would reduce the cost of lipid manufacture considerably.

There are three hazardous steps in the above sequences: the lithium aluminum hydride (LAH) reduction of methyl linoleate (step 1), the Grignard reaction (step 4) and the pyridinium chlorochromate (PCC) oxidation of the MC3 alcohol (step 6). The LAH and Grignard steps, while routinely carried out in pharmaceutical plants, have the attendant problem of elevated fire risk. These steps require scrupulously dry solvents, operation under oxygen- and water-free atmosphere, and careful work-up procedures. Step 6 involves the use of carcinogenic hexavalent chromium, which imposes significant effluent disposal costs.

(c) Synthesis Routes to Make Nor-KC2 and Nor-MC3 Overcome these Drawbacks Using Claisen Technology

The inventors have found that derivatives of MC3 and KC2 having C₁₇ chains instead of C₁₈ chains can be prepared by a shorter, more economical synthesis scheme that addresses the above issues. The inventors describe such derivatives as nor-KC2 and nor-MC3. The structures of the nor-lipids and their relationship to the original compounds are apparent from the diagram below:

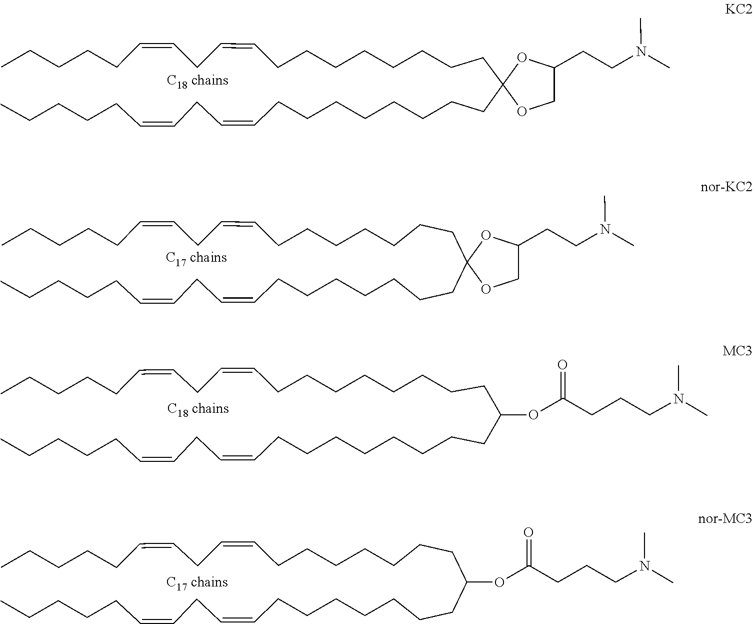

The synthesis of nor-KC2 requires ketone 7, while that of nor-MC3 requires alcohol 8. The nor-MC3 lipid can be prepared from ketone 7 by treatment with sodium borohydride (NaBH₄) in an appropriate solvent:

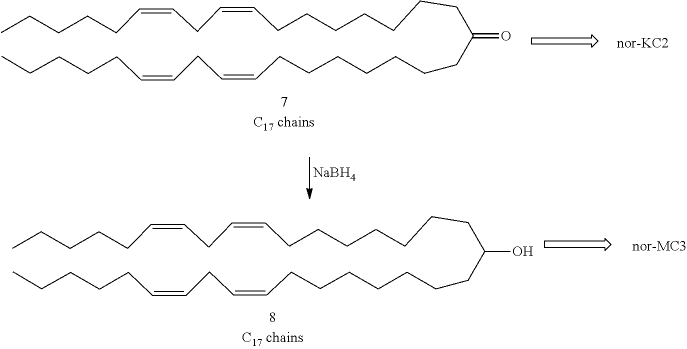 [SMILES: CCCCC/C=C\C/C=C\CCCCCCCC(=O)CCCCCCC/C=C\C/C=C\CCCCC ; "NaBH4" ; CCCCC/C=C\C/C=C\CCCCCCCC(O)CCCCCCC/C=C\C/C=C\CCCCC]

Therefore, the first objective of the inventors was to devise a method for the preparation of nor-KC2 ketone 7. The nor-KC2 ketone 7 is available by Claisen condensation of a linoleate ester, e.g., methyl linoleate, 5, followed by hydrolysis and decarboxylation of the resulting ketoester 6:

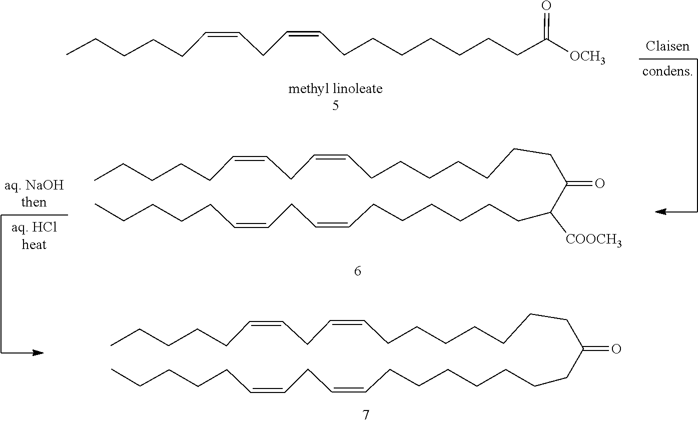 [SMILES: CCCCC/C=C\C/C=C\CCCCCCCC(=O)OC ; CCCCC/C=C\C/C=C\CCCCCCCC(=O)C(CCCCCC/C=C\C/C=C\CCCCC)C(=O)OC ; CCCCC/C=C\C/C=C\CCCCCCCC(=O)CCCCCCC/C=C\C/C=C\CCCCC]

However, the Claisen condensation is commonly carried out in the presence of strong bases (e.g., alkoxides or—especially—sodium hydride, NaH) at elevated temperatures (120-150 degrees C.). This is a significant drawback as polyunsaturated fatty acid derivatives like methyl linoleate are intolerant of such conditions, which tend to induce various degrees of double bond isomerization. The inventors have observed such isomerizations in the course of their own research.

Further, NaH, which is a particularly effective reagent for traditional Claisen condensation, poses safety hazards comparable to LAH and thus it is best avoided. Furthermore, the actual base that forms under the reported conditions is a sodium alkoxide, which is the very agent that is likely to induce double bond isomerization. Equally noteworthy is the fact that an analogous procedure can be employed for the Claisen condensation of methyl linolenate, a tri-unsaturated analogue of linoleate that is even more prone to base-mediated isomerization.

While di- and tri-unsaturated analogues are particularly prone to isomerization, the same problem may also arise with mono-unsaturated alkyl groups, such as in the case of the sturdier methyl oleate, which contains only one double bond.

Regardless, the present disclosure provides a synthetic route based on Claisen condensation that will preserve the double bonds intact. Such method involves the use of weakly basic agents (e.g., amines) at or near room temperature; e.g., from −10 to +40° C.

The inventors have found that such conditions promote a clean, efficient Claisen condensation of the more sensitive methyl linoleate to produce ketoester 6. This product is advanced to ketone 7 in a conventional manner.

Advantageously, this route to 7 bypasses the need for LAH, Grignard, or PCC and reaches the desired ketone in only two steps.

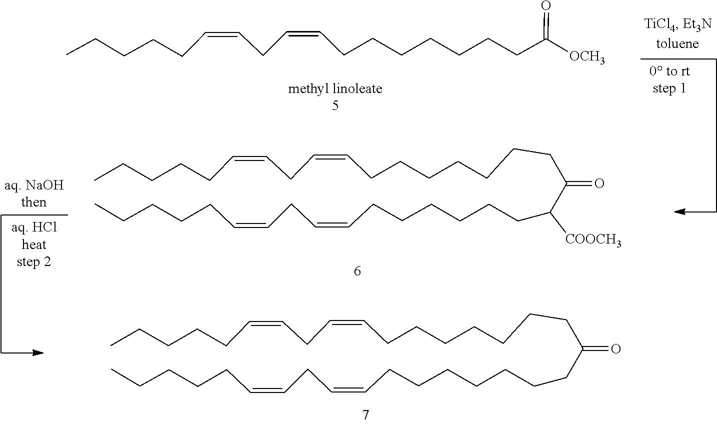 [SMILES: CCCCC/C=C\C/C=C\CCCCCCCC(=O)OC ; CCCCC/C=C\C/C=C\CCCCCCCC(=O)C(CCCCCC/C=C\C/C=C\CCCCC)C(=O)OC ; CCCCC/C=C\C/C=C\CCCCCCCC(=O)CCCCCCC/C=C\C/C=C\CCCCC]

Ketone 7 may be converted into nor-KC2 in 3 steps by same method used to make actual KC2:

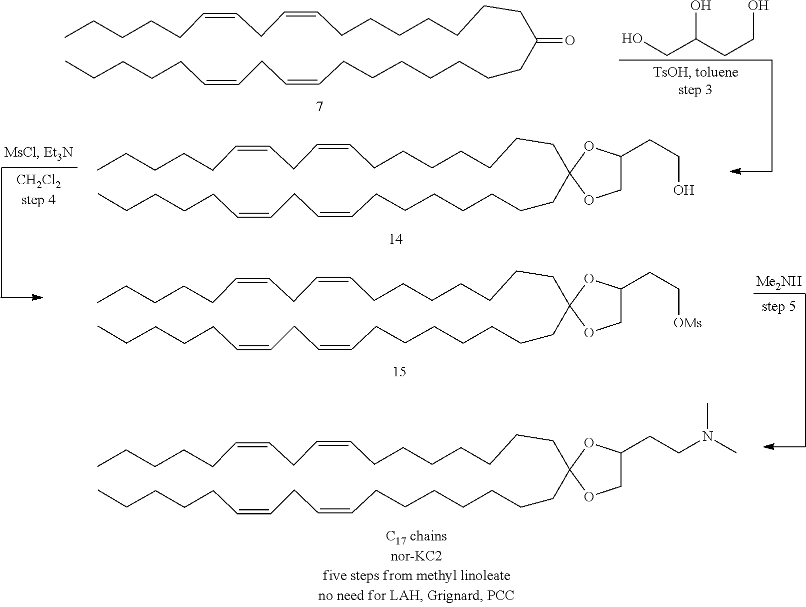 [SMILES: "Me2NH" ; CCCCC/C=C\C/C=C\CCCCCCCC(=O)CCCCCCC/C=C\C/C=C\CCCCC ; OCCC(O)CO ; CCCCC/C=C\C/C=C\CCCCCCCC1(CCCCCCC/C=C\C/C=C\CCCCC)OCC(CCO)O1 ; CCCCC/C=C\C/C=C\CCCCCCCC1(CCCCCCC/C=C\C/C=C\CCCCC)OCC(CCOS(C)(=O)=O)O1 ; CCCCC/C=C\C/C=C\CCCCCCCC1(CCCCCCC/C=C\C/C=C\CCCCC)OCC(CCN(C)C)O1]

or in one step by reaction with the hydrochloride of aminodiol 16:

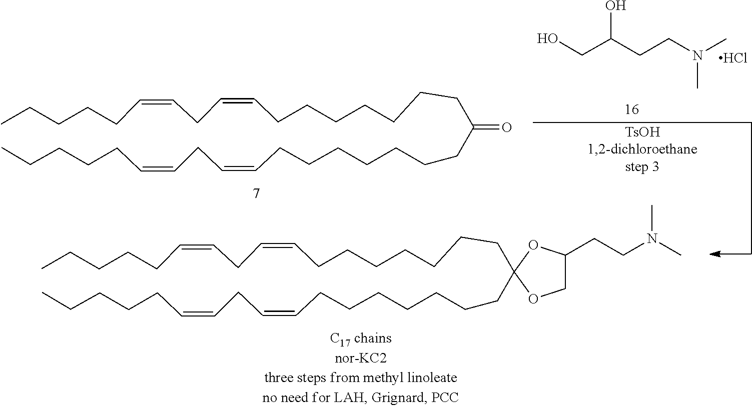

The assembly of nor-MC3 requires 2 steps from 7 (next page).

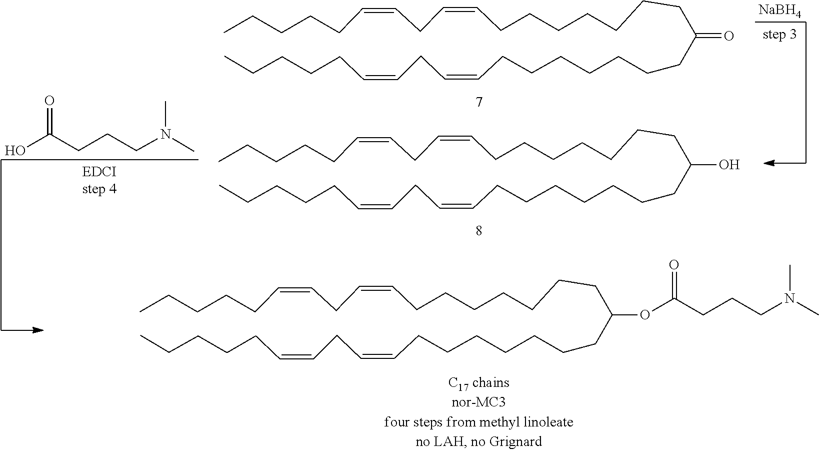

Thus, as will be evident from the above discussion, the new routes to nor-KC2 and nor-MC3 have fewer steps than those leading to the original KC2 (5 or 3 vs. 9 steps) and MC3 (4 vs. 6 steps) and bypass the need for LAH, Grignard, and PCC.

The foregoing example is provided to exemplify the synthetic route of the disclosure and its advantages over known methodologies to manufacture lipids using the synthesis of nor-KC2 and nor-MC3 as examples. As discussed previously, the method is more broadly applicable to the production of lipid classes besides nor-KC2 and nor-MC3.

### Example 2: Preparation of Starting Materials for Lipid Synthesis Routes

With reference to FIG. 1, the starting material for the Claisen condensation may be a fatty acid methyl ester 1. In Schemes A-F, H, J, and K the fatty acid methyl esters are readily obtained from commercial sources.

In Schemes G and I, the fatty esters were synthesized using the schemes set forth in this example.

In reaction Scheme G to prepare sulfur lipids, such as MF19, the material fed to the Claisen condensation is Methyl 9-(((pentylthio)methyl)thio)nonanoate, which is substituted with two sulfur atoms in its alkyl group:

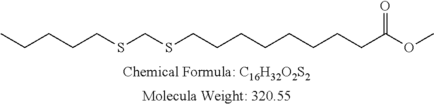 [SMILES: CCCCCSCSCCCCCCCCC(=O)OC]

In reaction Scheme I, the material fed to the Claisen condensation is Methyl 9,9-bis(octylthio)nonanoate. The alkyl portion of the fatty acid has two alkyl groups that converge at a central carbon atom distal from the methyl ester with adjacent sulfur atoms in each chain as shown below:

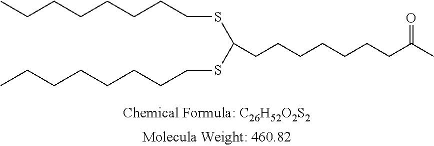 [SMILES: CCCCCCCCSC(CCCCCCCC(C)=O)SCCCCCCCC]

The preparation of these starting materials, among others, for the various synthetic schemes described herein is exemplified below. It will be understood by those of skill in the art, however, that other synthetic routes could be used to prepare these fatty acid methyl esters.

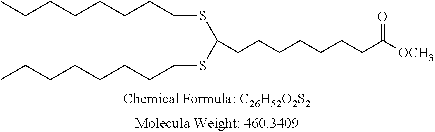 [SMILES: CCCCCCCCSC(CCCCCCCC(=O)OC)SCCCCCCCC]

Methyl 9,9-bis(octylthio)nonanoate. Concentrated sulfuric acid (18 M, two drops) was added to a cold (ice bath), stirred solution of methyl-9-oxononanoate (930 mg, 5 mmol) and octanethiol (1.6 g, 1.9 mL, 11 mmol, 1.1 equiv) in ether (5 mL). (The methyl-9-oxononanoate was prepared according to: Dunny, E.; Evans, P. J. Org. Chem. 2010, 75, 5334, incorporated herein by reference). The mixture was stirred for 1 h, during which time it was allowed to reach room temperature. At this point, analysis of the mixture by NMR indicated that the reaction was complete. The mixture was diluted with 1:1 ether-hexane (5 mL) and washed with 10% aq. NaOH solution, and subsequently it was passed over a plug of anhydrous Na₂SO₄ and evaporated. The oily residue was purified by silica gel column chromatography (5% EtOAc acetate in hexanes) to afford the pure product. 1H NMR δ 3.72 (t, 1H), 3.61 (s, 3H), 2.44 (t, 4H), 2.30 (t, 2H), 2.00-1.20 (m, 36H), 0.88 (t, 6H). 13C NMR δ 173.1, 52.1, 51.4, 36.2, 33.6, 31.9, 30.3, 29.4, 28.9, 28.5, 25.2, 25.0, 22.7, 14.1 (some peaks doubled). LRMS: m/z 483 [M+Na]⁺

Methyl 9-((methylsulfonyl)oxy)nonanoate. Neat methanesulfonyl chloride (685 mg, 463 uL,

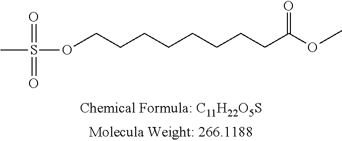

6.0 mmol, 1.1 equiv) was added dropwise (syringe) to a cold (ice bath) solution of methyl 9-hydroxynonanoate (1.03 g, 5.5 mmol) and triethylamine (667 mg, 920 uL, 6.6 mmol, 1.2 equiv) in THF (11 mL). (The methyl 9-hydroxynonanoate was prepared according to Dunny, E.; Evans, P. J. Org. Chem. 2010, 75, 5334, incorporated herein by reference). A white precipitate formed and after 45 min, the reaction was complete (TLC, NMR). Aqueous 1 M HCl solution was subsequently added (5 mL) and most of the THF was removed on a rotary evaporator. The aqueous residue was extracted with ether (3×15 mL). The combined extracts were washed with brine (10 mL), passed over a plug of anhydrous Na₂SO₄ and evaporated to afford 1.33 g (91%) of the mesylate, which was advanced to the next step without purification. 1H NMR δ 4.25 (t, 2H), 3.70 (s, 3H), 3.01 (s, 3H), 2.32 (t, 2H), 1.80-1.46 (m, 12H).

Methyl 9-(acetythio)nonanoate. Neat thiolacetic acid (500 mg, 465 ul, 6.5 mmol, 1.2 equiv)

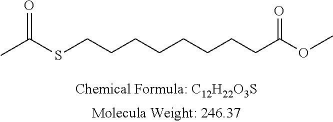 [SMILES: COC(=O)CCCCCCCCSC(C)=O]

was carefully added to a cold (0° C.), stirred suspension of NaH (400 mg of 60% dispersion in oil, pre-washed with pentane, 240 mg of NaH, 10 mmol, 1.5 equiv; vigorous reaction, H₂ evolved) in dry THF (5 mL), under argon. (Care should be taken due to H₂ evolution). When bubbling stopped, a solution of the above mesylate (1.45 g, 5.5 mmol, 1 equiv) in THF (5 mL) was added via syringe. The mixture was stirred overnight, during which time it was allowed to reach room temperature. After this time, TLC and NMR indicated that the reaction was complete. The mixture was diluted with 1:1 ether/hexanes (20 mL) and carefully quenched with DI water. (Care should be taken due to H₂ evolved). The phases were separated and the aqueous layer was extracted with more 1:1 ether-hexanes (2×10 mL). The combined extracts were washed with brine (15 mL), passed over a plug of anhydrous Na₂SO₄ and evaporated. The residue (1.3 g, 95%) was used directly for the next step. 1H NMR δ 3.69 (s, 3H), 2.87 (t, 2H), 2.33 (s, 3H), 2.30 (t, 2H), 1.64-1.26 (m, 12H).

General procedure for the preparation of chloromethyl-alkylsulfanes. Gaseous HCl was bubbled through a cold (ice-salt bath, −15° C.) solution of a thiol (10 mmol) in dry CH₂Cl₂ (10 mL) containing suspended paraformaldehyde (12 mmol) and maintained under argon (balloon; needle vent). The mixture was stirred for 2 hours at −15° C., then the solvent was removed under reduced pressure. The residue was taken up with 1:1 ether/hexanes (10 mL), and DI H₂O (5 mL) was added until all the solid residue disappeared. The phases were separated and the aqueous layer was extracted with more Et₂O (2×10 mL). The combined extracts were sequentially washed with saturated aqueous sodium bicarbonate solution (3×10 mL) and brine (2×10 mL), passed through a plug of anhydrous Na₂SO₄, and concentrated on a rotary evaporator to afford the chloromethyl alkyl sulfane, colorless oil, in essentially quantitative yield. This sensitive product was used without further purification. The following compounds were thus obtained:

(Chloromethyl)(pentyl)sulfane: 99% from pentanethiol. 1H NMR δ 4.77 (s, 2H), 2.76 (t, 2H),

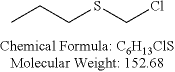

### 1.68 (m, 2H), 1.39 (m, 4H), 0.93 (t, 3H). 13C NMR δ 49.9, 31.6, 30.9, 28.3, 22.2, 13.9.

(Chloromethyl)(octyl)sulfane: ca. 100% from octanethiol. 1H NMR δ 4.75 (s, 2H), 2.74 (t, 2H),

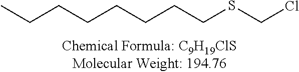

Methyl 9-(((pentylthio)methyl)thio)nonanoate. A stirred suspension of K₂CO₃ (828 mg, 6

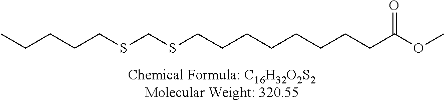 [SMILES: CCCCCSCSCCCCCCCCC(=O)OC]

mmol, 1.2 equiv) in dry methanol (8 mL) was thoroughly degassed with argon (additional methanol was added to maintain volume) prior to injection of a solution of methyl 9-(acetythio)nonanoate (1.2 g, 5 mmol) in a total of 5 mL of degassed dry methanol. The mixture was maintained under argon (balloon). After 45 min, a TLC analysis (capillary introduced into the flask through a 16-ga needle) indicated that no starting material was present. The balloon was replaced with an argon line and the solvent was removed by sweeping with argon (16-ga needle vent). When essentially no methanol remained, dry THF was added (8 mL) and the argon line was replaced with an argon balloon. A solution of (pentyl)(chloromethyl)sulfane (920 mg, 6 mmol, 1.2 equiv) in THF (total of 5 mL) was injected (syringe) and the well-stirred mixture was heated to 50° C. After 5 h, TLC and NMR indicated that the reaction was complete. The reaction was quenched with DI water (10 mL) and extracted with 1:1 ether-hexanes (3×10 mL). The combined extracts were sequentially washed with 10% aqueous NaOH solution (2×5 mL) and brine (5 mL), passed through a plug of anhydrous Na₂SO₄, and evaporated. The residue was purified by silica gel column chromatography (1% EtOAc/hexanes) to afford 1.4 g (87%) of methyl 9-(((pentylthio) methyl)thio)nonanoate as a clear, colorless liquid. 1H NMR δ 3.68 (s, 3H), 3.66 (s, 2H), 2.62 (t, 4H), 2.33 (t, 2H), 1.64-1.25 (m, 18H), 0.89 (t, 3H). 13C NMR δ 173.2, 52.0, 37.9, 33.6, 32.1, 30.7, 29.9, 29.6, 29.3, 29.0, 28.9, 28.5, 25.0, 22.0, 14.2. LRMS: m/z 321 [M+H]⁺, 343 [M+Na]⁺

Methyl 9-(((octylthio)methyl)thio)nonanoate. Obtained as a colorless oil in 90% yield by the

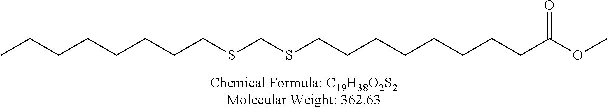 [SMILES: CCCCCCCCSCSCCCCCCCCC(=O)OC]

procedure described above by using (chloromethyl)(octyl)sulfane in lieu of (chloromethyl)(pentyl)sulfane. 1H NMR δ 3.68 (s, 3H), 3.66 (s, 2H), 2.62 (t, 4H), 2.33 (t, 2H), 1.65-1.25 (m, 18H), 0.89 (t, 3H). ¹³C NMR δ 173.2, 52.0, 37.9, 33.6, 32.1, 31.9, 29.9, 29.4, 29.3, 29.0, 28.9, 28.5, 25.0, 22.7, 14.2 (some signals overlap). LRMS: m/z 363 [M+H]⁺, 385 [M+Na]⁺.

### Example 3: General Procedure for Claisen Condensation

This example describes a Claisen condensation of a fatty acid methyl ester 1 to a corresponding ketoester 2 (see FIG. 1) carried out under mild conditions in accordance with an embodiment of the disclosure.

A solution of TiCl₄ (9.6 g, 5.7 mL, 45.0 mmol) in toluene (12 mL) was added dropwise to a cold (0° C., ice bath), stirred solution of an appropriate methyl ester (30.0 mmol) and tributylamine (Bu₃N) (10.2 g, 12.9 mL, 54.0 mmol) in toluene (50.0 mL). After stirring at 0° C. for 1.5 h, the reaction was complete as determined by TLC and ¹H NMR. The reaction solution was then diluted with hexanes (60 mL) and water (60 mL) was cautiously added. Addition of water caused evolution of heat, so the temperature of the mixture was controlled by thorough stirring and cooling in an ice bath. The organic phase was separated and the aqueous phase was extracted with more hexane (2×40 mL). The combined organic extracts were washed with water, passed over a plug of anhydrous Na₂SO₄ and concentrated under vacuum. Proton NMR analysis of the residue indicated the presence of some residual toluene. Suspended inorganic matter(likely TiO₂) may also be present. The crude product may be purified by column chromatography (3% diethyl ether in hexanes) to afford pure ketoester (90-96%), but may be advanced directly to the next step. NMR indicated that the product existed as a mixture of keto (major) and enol derivatives, typically in a 2:1 ratio. The following ketoesters were thus obtained:

Methyl 2-dodecyl-3-oxohexadecanoate. 96% from methyl myristate, ca. 2:1 mixture of keto

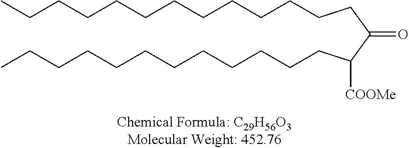 [SMILES: CCCCCCCCCCCCCC(=O)C(CCCCCCCCCCCC)C(=O)OC]

(major) and enol (minor) forms. 1H NMR (keto form) δ 3.65 (s, 3H), 3.31 (t, 1H), 2.43 (m, 2H), 2.0-1.2 (m, 44H), 0.88 (t, 6H). LRMS: m/z 453 [M+H]⁺, 475 [M+Na]⁺.

Methyl (Z)-2-((Z)-dodec-7-en-1-yl)-3-oxohexadec-11-enoate. 93% from methyl myristoleate,

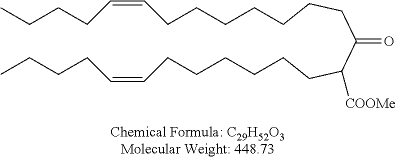 [SMILES: CCCC/C=C\CCCCCCCC(=O)C(CCCCCC/C=C\CCCC)C(=O)OC]

ca. 3:1 mixture of keto (major) and enol (minor) forms. ¹H NMR (keto form) δ 5.35 (m, 4H), 3.71 (s, 3H), 3.43 (t, 1H), 2.61-2.39 (m, 2H), 2.07-1.95 (m, 8H), 1.90-1.74 (m, 2H), 1.64-1.50 (m, 2H), 1.40-1.17 (m, 24H), 0.89 (t, 6H). 13C NMR (keto form) δ 205.6, 170.6, 130.2, 130.1, 52.4, 42.0, 32.1, 29.9, 29.8, 29.8, 29.7, 29.5, 29.4, 29.4, 29.3, 29.2, 29.1, 28.4, 27.7, 27.4, 23.5, 22.8, 14.2 (some peaks are doubled).

LRMS: m/z 449 [M+H]⁺, 471 [M+Na]⁺.

Methyl (Z)-2-((Z)-hexadec-7-en-1-yl)-3-oxoicos-11-enoate. 95% from methyl oleate, ca. 2:1

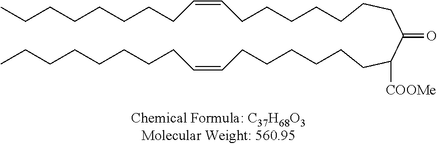 [SMILES: CCCCCCCC/C=C\CCCCCCCC(=O)C(CCCCCC/C=C\CCCCCCCC)C(=O)OC]

mixture of keto (major) and enol (minor) forms. 1H NMR (keto form) δ 5.35 (m, 4H), 3.71 (s, 3H), 3.43 (t, 1H), 2.61-2.39 (m, 2H), 2.07-1.95 (m, 8H), 1.90-1.74 (m, 2H), 1.64-1.50 (m, 2H), 1.40-1.17 (m, 40H), 0.89 (t, 6H). 13C NMR (keto form) δ 205.6, 170.6, 130.2, 130.1, 129.9, 129.8, 59.2, 52.4, 42.0, 32.1, 29.9, 29.8, 29.8, 29.7, 29.5, 29.4, 29.4, 29.3, 29.2, 29.1, 28.4, 27.6, 27.4, 27.3, 27.3, 23.6, 22.8, 14.3. LRMS: m/z 561 [M+H]⁺, 583 [M+Na]⁺

Methyl (11Z,14Z)-2-((7Z,10Z)-hexadeca-7,10-dien-1-yl)-3-oxoicosa-11,14-dienoate. 96%

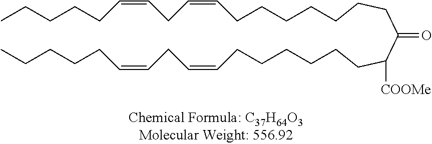 [SMILES: CCCCC/C=C\C/C=C\CCCCCCCC(=O)C(CCCCCC/C=C\C/C=C\CCCCC)C(=O)OC]

from methyl linoleate, ca. 2:1 mixture of keto (major) and enol (minor) forms. H NMR (keto form) δ 5.37 (m, 8H), 3.77 (s, 3H), 3.45 (t, 1H), 2.79 (t, 4H), 2.40 (t, 2H), 2.07 (m, 8H), 1.85-1.20 (m, 32H), 0.91 (t, 6H). 13C NMR (keto form) δ 205.6, 170.6, 130.2, 130.1, 129.9, 129.8, 59.2, 52.4, 42.0, 32.1, 29.9, 29.8, 29.8, 29.7, 29.5, 29.4, 29.4, 29.3, 29.2, 29.1, 28.4, 27.6, 27.4, 27.3, 27.3, 23.6, 22.8, 14.3 (some peaks are doubled). LRMS: m/z 557 [M+H]⁺, 579 [M+Na]⁺

Methyl (11Z,14Z,17Z)-2-((7Z,10Z,13Z)-hexadeca-7,10,13-trien-1-yl)-3-oxoicosa-11,14,17-trienoate. 94% from methyl linolenate, ca. 2:1 mixture of keto (major) and enol (minor) forms.

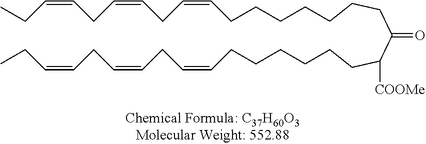 [SMILES: CC/C=C\C/C=C\C/C=C\CCCCCCCC(=O)C(CCCCCC/C=C\C/C=C\C/C=C\CC)C(=O)OC]

¹H NMR (keto form) δ 5.50-5.25 (m, 12H), 3.69 (s, 3H), 3.48 (t, 1H), 2.82 (m, 8H), 2.43 (t, 2H), 2.15 (m, 4H), 2.04 (m, 4H), 1.92-1.20 (m, 20H), 0.81 (t, 6H). LRMS: m/z 553 [M+H]⁺, 575 [M+Na]⁺.

Methyl 2-(7,7-bis(octylthio)heptyl)-11,11-bis(octylthio)-3-oxoundecanoate. 95% from methyl

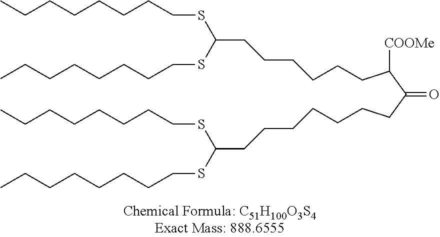 [SMILES: CCCCCCCCSC(CCCCCCCC(=O)C(CCCCCCC(SCCCCCCCC)SCCCCCCCC)C(=O)OC)SCCCCCCCC]

9,9-bis(octylthio)nonanoate (obtained as described on p. 1); ca. 3:1 mixture of keto (major) and enol (minor) forms. 1H NMR (keto form) δ 3.75 (t, 2H), 3.70 (s, 3H), 3.44 (t, 1H), 2.74-2.49 (m, 8H), 2.44 (t, 2H), 1.92-1.20 (m, 78H), 0.88 (t, 6H). LRMS: m/z 889 [M+H]⁺, 911 [M+Na]⁺Methyl 3-oxo-11-(((pentylthio)methyl)thio)-2-(7-(((pentylthio)methyl)thio)heptyl)undecano-ate. 95% from methyl 9-(((pentylthio)methyl)thio)-nonanoate (prepared as described on p. 4); ca, 2:1 mixture of keto and enol isomers. 1H NMR

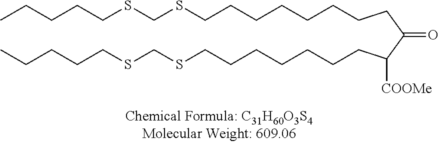 [SMILES: CCCCCSCSCCCCCCCCC(=O)C(CCCCCCCSCSCCCCC)C(=O)OC]

(keto form) δ 3.68 (s, 4H), 3.70 (s, 3H), 3.40 (t, 1H), 2.64 (m, 8H), 2.43 (t, 2H) 1.92-1.31 (m, 36H), 0.92 (t, 6H). LRMS: m/z 609 [M+H]⁺, 631 [M+Na]⁺

### Example 4. General Procedure for Hydrolysis and Decarboxylation of the Claisen Product to Produce the Ketone

The following example describes the experimental procedure to produce a ketone 3 from base hydrolysis and decarboxylation of the ketoester 2 Claisen products (see FIG. 1) produced in Example 3.

Aqueous 10% w/vol NaOH (5 mL) was added to a solution of the above crude ketoester (5.0 g) in 95% ethanol (25 mL). The mixture was stirred at room temperature overnight. The reaction was checked for completion by adding 3-4 drops of the reaction mixture to 3 N aqueous HCl solution (0.5 mL), extracting the mixture with hexanes, evaporating the combined extracts to dryness, and checking the residue by ¹H NMR. The disappearance of the OCH₃ signal and a downfield shift of the triplet at 3.45 (ketoester) to 3.51 (ketoacid) indicated that the reaction was complete. The reaction mixture was concentrated on a rotary evaporator to remove ethanol. The aqueous residue was cooled in an ice bath, diluted with hexanes (60 mL), and vigorously stirred during careful dropwise addition of conc. aqueous HCl solution (heat evolved). When the mixture attained pH˜ 1, the phases were separated and the aqueous layer was extracted with more hexanes (2×20 mL). The combined organic extracts were washed with DI water (30 mL), passed over a plug of anhydrous Na₂SO₄, and concentrated on the rotary evaporator. An NMR spectrum of the crude product was recorded to ascertain the presence of the desired ketoacid.

The flask containing the residue from the rotary evaporation was capped with a septum and thoroughly purged with argon (balloon; needle vent). The flask was heated with a heat gun (while still sealed under argon and vented with a needle) until uncomfortably hot to the touch (100-130° C.), whereupon decarboxylation started. Bubbling of the residue was noticeable as the decarboxylation reaction proceeded. After approximately 10 min, no further bubbling was evident. The flask was cooled to room temperature and the residue was again analyzed by ¹H NMR, which revealed it to be nearly pure ketone. If desired, the crude ketone may be purified by column chromatography (gradient 1→3% v/v ether in hexanes). The crude ketone, however, is most advantageously introduced directly to the next steps.

The following ketones were thus obtained:
(9Z,26Z)-Pentatriaconta-9,26-dien-18-one. 1H NMR δ 5.40-5.28 (m, 4H), 2.37 (t, 4H), 2.05-1.95

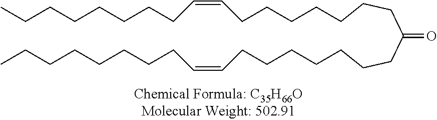 [SMILES: CCCCCCCC/C=C\CCCCCCCC(=O)CCCCCCC/C=C\CCCCCCCC]

(m, 8H), 1.62-1.49 (m, 4H), 1.40-1.19(m, 40H), 0.90 (t, 6H). 13C NMR δ 211.8, 130.1, 129.9, 43.0, 32.1, 29.9, 29.8, 29.7, 29.5, 29.4, 29.3, 27.4, 27.3, 24.0, 22.8, 14.3. LRMS: m/z 503 [M+H]⁺, 525 [M+Na]⁺.
(6Z,9Z,26Z,29Z)-Pentatriaconta-6,9,26,29-tetraen-18-one. H NMR δ 5.32 (m, 8H), 2.74 (t,

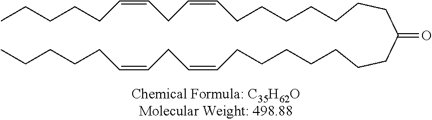 [SMILES: CCCCC/C=C\C/C=C\CCCCCCCC(=O)CCCCCCC/C=C\C/C=C\CCCCC]

4H), 2.35 (t, 4H), 2.02 (m, 8H), 1.55-1.20 (m, 28H), 0.87 (t, 6H). 13C NMR δ 210.9, 130.0, 129.8, 128.0, 127.8, 42.6, 31.4, 29.5, 29.24, 29.22, 29.1, 29.0, 27.0 (2 overlapping peaks), 25.5, 23.7, 22.5, 14.0. LRMS m/z 499 [M+H], 521 [M+Na]⁺.
(3Z,6Z,9Z,26Z,29Z,32Z)-Pentatriaconta-3,6,9,26,29,32-hexaen-18-one. 1H NMR δ 5.43-5.28

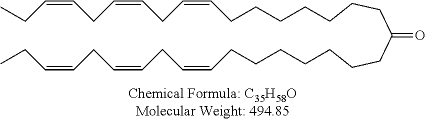 [SMILES: CC/C=C\C/C=C\C/C=C\CCCCCCCC(=O)CCCCCCC/C=C\C/C=C\C/C=C\CC]

(m, 12H), 2.83-2.74 (m, 8H, m), 2.39 (t, 4H), 2.19-2.00 (m, 8H), 1.60-1.52 (m, 4H), 1.40-1.22 (m, 16H), 0.98 (t, 6H). LRMS m/z 495 [M+H]⁺, 517 [M+Na]⁺.
1,1,17,17-Tetrakis(octylthio)heptadecan-9-one. 1H NMR δ 3.72 (t, 2H); 2.60 (m, 8H), 2.43 (t,

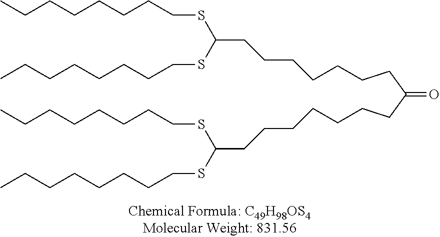 [SMILES: CCCCCCCCSC(CCCCCCCC(=O)CCCCCCCC(SCCCCCCCC)SCCCCCCCC)SCCCCCCCC]

4H), 2.05-1.20 (m, 72H), 0.87 (t, 12H). LRMS: m/z 831 [M+H]⁺, 853 [M+Na]⁺

6,8,26,28-Tetrathiatritriacontan-17-one. 1H NMR δ 3.64 (s, 4H), 2.65 (m, 8H), 2.42 (t, 4H),

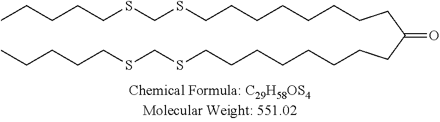 [SMILES: CCCCCSCSCCCCCCCCC(=O)CCCCCCCCSCSCCCCC]

### 1.60-1.20 (m, 36H), 0.90 (t, 6H). 13C NMR δ 210.8, 43.1, 37.5, 35.4, 32.1, 30.8, 29.6, 29.5, 29.2, 29.1, 28.9, 28.8, 25.7, 22.3, 13.9. LRMS: m/z 551 [M+H]⁺

### Example 5: Representative Procedure for Claisen Condensation/Saponification/Desilylation/Decarboxylation of Silyl Ether Derivatives of Fatty Hydroxyesters

Methyl (R,Z)-12-((tert-butyldimethtylsilyl)oxy)octadec-9-enoate. To a solution of methyl ricinoleate (90% pure, 13.0 g, 37.4 mmol)

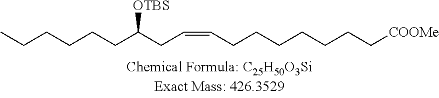 [SMILES: CCCCCC[C@H](C/C=C\CCCCCCCC(=O)OC)O[Si](C)(C)C(C)(C)C]

imidazole (3.7 g, 52.4 mmol) in CH₂Cl₂ (70.0 mL) was added TBSCl (6.8 g, 44.9 mmol) at 0° C. The mixture was warmed to room temperature and stirred for 18 hours, diluted with water (50.0 mL) and extracted with CH₂Cl₂ (3×60.0 mL). The combined organics were dried (Na₂SO₄) and concentrated to yield crude xx (15.73 g), which was used directly in the next step without further purification. 1H NMR (400 MHz, CDCl₃): δ 5.48-5.33 (m, 2H), 3.66 (s, 3H), 3.66-3.60 (m, 1H), 2.30 (t, J=7.6 Hz, 2H), 2.18 (t, J=5.9 Hz, 2H), 2.08-1.94 (m, 2H), 1.61 (q, J=7.3 Hz, 2H), 1.49-1.20 (m, 21H), 0.88 (s, 9H), 0.04 (d, J=1.3 Hz, 6H).

(7R,9Z,26Z,29R)-7,29-dihydroxypentatriaconta-9,26-dien-18-one. To a solution of methyl (R,Z)-12-((tert-butyldimethylsilyl)oxy)octadec-9-enoate (15.73 g, crude) and NBu₃ (16.6 mL,

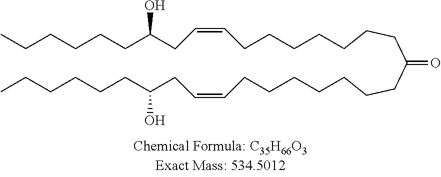 [SMILES: CCCCCC[C@@H](O)C/C=C\CCCCCCCC(=O)CCCCCCC/C=C\C[C@H](O)CCCCCC]

70.0 mmol) in toluene (50.0 mL) was added dropwise a solution of TiCl₄ (6.50 mL, 59.0 mmol) in toluene (20.0 mL) at 0° C. over the course of 20 minutes, after which point the mixture was warmed to room temperature and stirred for 1 hour. The reaction was then quenched with water at 0° C., and extracted with toluene (3×70.0 mL). The combined organics were concentrated. The residue was redissolved in EtOH (50.0 mL) and 4 N NaOH (20.0 mL) was added. The mixture was stirred for 18 hours, concentrated to ˜50% volume, acidified to pH 2 with conc. HCl and extracted with 50:50 Et₂O/Hexanes (3×75.0 mL). The combined organics were concentrated (note 1) and the residue was redissolved in EtOH (70 mL) and treated with TFA (7.50 mL. Note: the ¹H NMR spectrum of the crude produce before TFA treatment showed only partial removal of TBS group). The mixture was stirred for 2 hours, diluted with water and extracted with 50:50 Et20/Hexanes (3×75.0 mL). The combined organics were washed (brine), dried (Na₂SO₄) and concentrated. The residue was purified by silica chromatography (0-10% EtOAc in Hexanes) to yield ketone xx (8.00 g, 80% over 2 steps). 1H NMR (400 MHz, CDCl₃): δ 3.70-3.58 (m, 2H), 3.50 (s, 3H), 2.38 (t, J=7.4 Hz, 4H), 2.22 (ddd, J=7.5, 6.1, 1.5 Hz, 4H), 2.08-1.97 (m, 4H), 1.66-1.18 (m, 41H), 0.97 (t, J=7.4 Hz, 6H).

### Example 6: General Procedure for Ketalization of the Ketone

Ketalization of the ketone 3 was carried out using the following experimental steps.

A mixture of ketone (1.0 mmol), 1,2,4-butanetriol (technical grade, ca. 90%, 236 mg, 2 mmol) and pyridinium p-toluenesulfonate (50 mg, 0.2 mmol) in 50 mL of toluene was refluxed under nitrogen overnight with continuous removal of water (Dean-Stark trap). Upon completion of the reaction, the mixture was cooled to room temperature, washed with water (2×30 mL), dried by passing over a plug of anhydrous Na₂SO₄ and evaporated. The yellowish oily residue (0.6 g) was purified by silica gel (230-400 mesh, 50 g) column chromatography, with dichloromethane as eluent, to afford 0.87-0.93 mmol (87-93%) of pure ketal. The following compounds were thus obtained:

2-(2,2-Di((8Z,11Z)-heptadeca-8,11-dien-1-yl)-1,3-dioxolan-4-yl)ethan-1-ol. 1H NMR δ 5.36

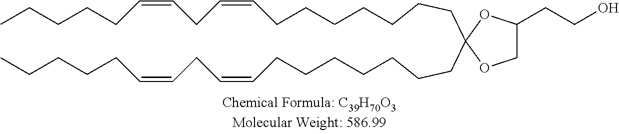 [SMILES: CCCCC/C=C\C/C=C\CCCCCCCC1(CCCCCCC/C=C\C/C=C\CCCCC)OCC(CCO)O1]

(m, 8H), 4.24 (m, 1H), 4.09 (m, 1H), 3.81 (m, 2H), 3.53 (t, 1H), 2.78 (t, 4H), 2.21 (t, 1H [OH]), 2.05 (m, 8H), 1.82 (m, 2H), 1.65-1.53 (m, 4H), 1.42-1.23 (m, 32H), 0.89 (t, 6H). C NMR δ 130.2, 130.1, 128.0, 127.9, 112.6, 75.5, 69.9, 60.9, 37.8, 37.3, 35.4, 31.5, 29.9, 29.7, 29.5, 29.34, 29.27, 27.22, 27.19, 25.6, 24.0, 23.8, 22.6, 14.1. LRMS: m/z 587 [M+H]⁺, 609 [M+Na]⁺

2-(2,2-Bis(8-(((pentylthio)methyl)thio)octyl)-1,3-dioxolan-4-yl)ethan-1-ol. 1H NMR δ 4.24

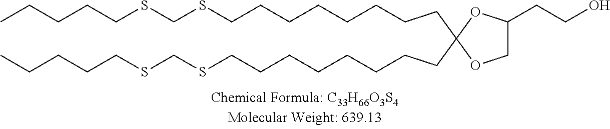 [SMILES: CCCCCSCSCCCCCCCCC1(CCCCCCCCSCSCCCCC)OCC(CCO)O1]

(m, 1H), 4.09 (m, 1H), 3.81 (m, 2H), 3.64 (s, 4H), 3.53 (t, 1H), 2.65 (m, 8H), 2.50 (br, 1H), 1.82 (m, 2H), 1.55-1.23 (m, 40H), 0.88 (t, 6H). 13C NMR δ 112.6, 75.5, 69.9, 60.9, 37.8, 37.3, 35.4, 31.5, 29.9, 29.7, 29.5, 29.4, 29.3, 27.2, 25.6, 24.0, 23.8, 22.6, 14.1. LRMS: m/z 661 [M+Na]⁺

### Example 7: General Procedure for Ketal-Alcohol Mesylation

Neat methanesulfonyl anhydride (290 mg, 1.6 mmol) was added to a solution of a ketal alcohol (prepared according to the previous section; 0.8 mmol) and dry triethylamine (242 mg, 330 uL, 2.4 mmol) in 5 mL of dry CH₂Cl₂. The resulting mixture was stirred at room temperature overnight. The mixture was diluted with 25 mL of CH₂Cl₂. the organic phase was washed with water (2×30 mL), passed over a plug of anhydrous Na₂SO₄, and evaporated to afford 510 mg of mesylate as yellowish oil. The crude mesylate was used in the following step without further purification. The following compounds were thus obtained:

2-(2,2-di((8Z,11Z)-Heptadeca-8,11-dien-1-yl)-1,3-dioxolan-4-yl)ethyl methanesulfonate.

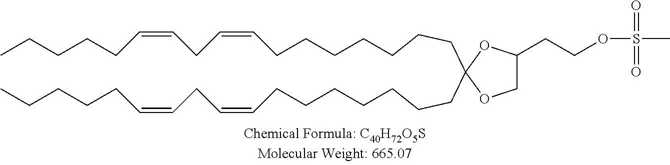 [SMILES: CCCCC/C=C\C/C=C\CCCCCCCC1(CCCCCCC/C=C\C/C=C\CCCCC)OCC(CCOS(C)(=O)=O)O1]

¹H NMR δ 5.34 (m, 8H), 4.35(m, 2H), 4.18 (m, 1H), 4.08 (m, ¹H), 3.52 (t, 1H), 3.01 (s, 3H), 2.76 (t, 4H), 2.01 (m, 10H), 1.58-1.20 (m, 36H), 0.88 (t, 6H). 13C NMR δ 130.1, 130.0, 127.9, 127.8, 112.6, 72.2, 69.5, 67.1, 37.6, 33.3, 31.5, 31.4, 29.83, 29.81, 29.6, 29.5, 29.3, 29.21, 29.19, 27.2, 27.1, 25.6, 24.0, 23.7, 22.5, 14.0. LRMS: m/z 665 [M+H]⁺, 687 [M+Na]⁺.

2-(2,2-bis(8-(((pentylthio)methyl)thio)octyl)-1,3-dioxolan-4-yl)ethyl methanesulfonate

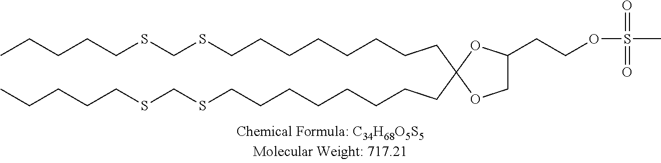 [SMILES: CCCCCSCSCCCCCCCCC1(CCCCCCCCSCSCCCCC)OCC(CCOS(C)(=O)=O)O1]

¹H NMR δ 4.35 (m, 2H), 4.17 (m, 1H), 4.08 (m, 1H), 3.52 (t, 1H), 3.68 (s, 4H), 3.01 (s, 3H), 2.64 (m, 8H), 1.95 (m, 2H), 1.68-1.25 (m, 40H), 0.92 (t, 6H). LRMS: m/z 717 [M+H]⁺, 739 [M+Na]⁺.

### Example 8: General Procedure for Dimethylaminolysis of the Mesylate

The above crude mesylate (500 mg) was added to 20 mL of a commercial 2.0 M solution of dimethylamine in THF. The resulting mixture was stirred at room temperature for 6 days, whereupon no more mesylate was apparent by TLC and/or ¹H NMR. Evaporation of the volatiles returned an oily residue that was purified by column chromatography on silica gel (230-400 mesh, 50 g) with 0-5% methanol gradient in dichloromethane as eluent, resulting in recovery of 350-400 mg of pure product. The following compounds were thus obtained:

2-(2,2-Di((8Z,11Z)-heptadeca-8,11-dien-1-yl)-1,3-dioxolan-4-yl)-N,N-dimethylethan-1-amine. 1H NMR δ 5.36 (m, 8), 4.07 (m, 2H), 3.49 (t, 1H), 2.78 (t, 4H), 2.46-2.24 (m, 2H), 2.23

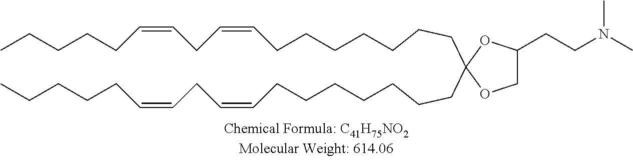 [SMILES: CCCCC/C=C\C/C=C\CCCCCCCC1(CCCCCCC/C=C\C/C=C\CCCCC)OCC(CCN(C)C)O1]

(s, 6H), 2.06 (m, 8H), 1.89-1.59 (m, 2H), 1.58 (m, 4H), 1.42-1.20 (m, 32H), 0.90 (br t, 6H). C NMR δ 130.1 (2 signals), 127.9 (2 signals), 112.1, 74.7, 69.9, 56.3, 45.4, 37.8, 37.5, 31.8, 31.5, 29.9 (2 signals), 29.7, 29.6 (2 signals), 29.5 (2 signals), 29.3 (2 signals), 27.2 (2 signals), 25.6, 24.0, 23.7, 22.6, 14.1. LRMS: m/z 614 [M+H]⁺

2-(2,2-Bis(8,8-bis(octylthio)octyl)-1,3-dioxolan-4-yl)-N,N-dimethylethan-1-amine

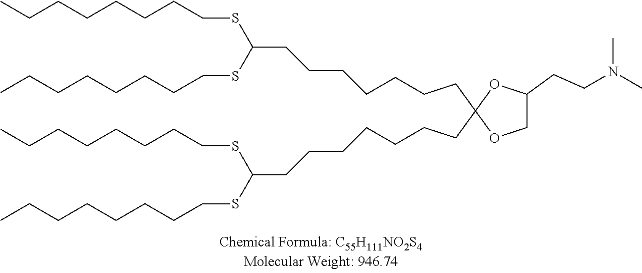 [SMILES: CCCCCCCCSC(CCCCCCCC1(CCCCCCCC(SCCCCCCCC)SCCCCCCCC)OCC(CCN(C)C)O1)SCCCCCCCC]

### ¹H NMR δ H NMR δ 4.07 (m, 2H), 3.75 (t, 2H), 3.49 (t, 1H), 2.74-2.49 (m, 8H), 2.46-2.24 (m, 2H), 2.23 (s, 6H), 1.89-1.59 (m, 2H), 1.58 (m 4H) 1.42-1.20 (m 78H) 0.90 (br t, 6H). LRMS: m/z 946 [M+H]

2-(2,2-Bis(8-(((pentylthio)methyl)thio)octyl)-1,3-dioxolan-4-yl)-N,N-dimethylethan-1-amine

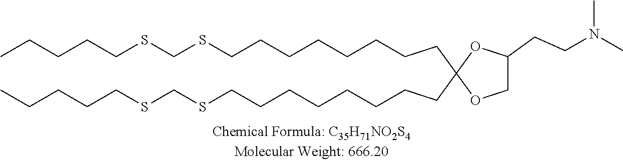 [SMILES: CCCCCSCSCCCCCCCCC1(CCCCCCCCSCSCCCCC)OCC(CCN(C)C)O1]

¹H NMR δ 4.07 (m, 2H), 3.68 (s, 4H), 3.49 (t, 1H), 2.64 (m, 8H), 2.46-2.24 (m, 2H), 2.23 (s, 6H), 2.06 (m, 8H), 1.89-1.59 (m, 2H), 1.58 (m, 4H), 1.42-1.20 (m, 30H), 0.90 (br t, 6H). 13C NMR δ 112.1, 74.7, 69.9, 56.3, 45.4, 37.8, 37.5, 35.4, 31.8, 31.1, 30.8, 31.5, 29.6, 29.5, 29.2, 29.1, 28.9, 28.8, 25.7, 22.3, 13.9. LRMS: m/z 666 [M+H]⁺

### Example 9: General Procedure for Reductive Amination of the Ketone

4-Aminobutanol, tert-butyldiphenylsilyl ether. A solution of tert-butyl(chloro)-diphenylsilane (TBDPSCl; 6.8 g, 24.7 mmol, 1.1 equiv) in CH₂Cl₂ (4 mL) was added dropwise during 15 min to

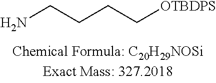

a well stirred solution of 4-amino-1-butanol (2.0 g, 22.4 mmol 1.0 equiv) and imidazole (3.4 g, 49.3 mmol, 2.2 equiv) in DCM (5 mL). The mixture was stirred overnight at room temperature. The reaction mixture was sequentially washed with sat. aq. NaHCO₃ solution (2×5 mL), water (2×5 mL), and sat. aq. NaCl chloride solution (2×5 mL), then dried over anhydrous Na₂SO₄, filtered, and concentrated under reduced pressure to furnish 7 (6.72 g, 92%) as a yellow oil. 1H NMR (300 MHz, CDCl₃): δ=7.71-7.68 (m, 4H), 7.40-7.36 (m, 6H), 3.70 (t, J=6.0 Hz, 2H), 2.67 (t, J=6.6 Hz, 2H), 1.86 (s, 2H), 1.65-1.48 (m, 4H), 1.09 (s, 9H); 13C NMR (75 MHz, CDCl₃): δ=135.4, 133.8, 129.4, 127.5, 63.6, 41.8, 29.9, 29.8, 26.7, 19.0.

Reductive amination of the ketone: (6Z,9Z,26Z,29Z)-N-(4-((tert-butyldiphenylsilyl)oxy)butyl)pentatriaconta-6,9,26,29-tetraen-18-amine.

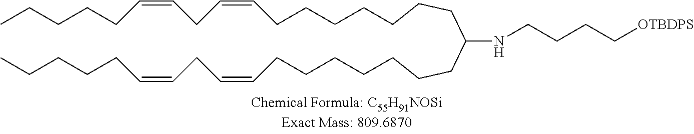

To a solution of (6Z,9Z,26Z,29Z)-pentatriaconta-6,9,26,29-tetraen-18-one (100 mg, 0.200 mmol) and TBDPS-protected 1-aminobutanol (112 mg, 0.341 mmol) in DCE (1.50 mL) was added sequentially NaBH(OAc)₃ (63.6 mg, 0.300 mmol) and glacial acetic acid (0.0250 mL). The reaction was stirred at room temperature for 18 hours then quenched with sat. NaHCO₃(4.00 mL) and extracted with DCM (3×4.00 mL). The combined organics were dried (Na₂SO₄) and concentrated. The residue was purified by silica chromatography (0-5% MeOH in DCM) to yield (6Z,9Z,26Z,29Z)-N-(4-((tert-butyldiphenylsilyl)oxy)butyl)pentatriaconta-6,9,26,29-tetraen-18-amine as an oil (120 mg, 74%). ¹H NMR (400 MHz, CDCl₃): δ 7.77-7.57 (m, 4H), 7.51-7.34 (m, 6H), 5.58-5.15 (m, 8H), 3.75-3.63 (m, 2H), 2.94 (s, 3H), 2.79 (t, J=6.7 Hz, 4H), 2.16-1.99 (m, 8H), 1.82-1.49 (m, 10H), 1.48-1.20 (m, 30H), 1.10-1.03 (m, 9H), 0.91 (t, J=6.8 Hz, 6H).

Reductive methylation of 4-(((6Z,9Z,26Z,29Z)-pentatriaconta-6,9,26,29-tetraen-18-yl)amino)butan-1-ol: (6Z,9Z,26Z,29Z)-N-(4-((tert-butyldiphenylsilyl)oxy)butyl)-N-methylpentatriaconta-6,9,26,29-tetraen-18-amine

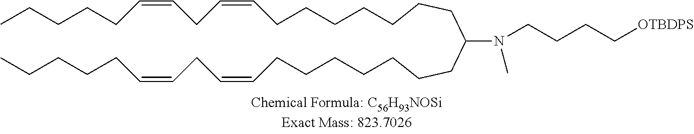

To a solution of (6Z,9Z,26Z,29Z)-N-(4-((tert-butyldiphenylsilyl)oxy)butyl)pentatriaconta-6,9,26,29-tetraen-18-amine. (120 mg, 0.148 mmol) in THF (2.00 mL) and formaldehyde (37 wt. % in water, 1.00 mL) was added NaBH(OAc)₃ (157 mg, 0.740 mmol). The mixture was stirred at room temperature for 3 days then quenched with sat. NaHCO₃ (4.00 mL) and extracted with DCM (3×4.00 mL). The combined organics were dried (Na₂SO₄) and concentrated. The residue was purified by silica chromatography (0-5% MeOH in DCM) to yield (6Z,9Z,26Z,29Z)-N-(4-((tert-butyldiphenylsilyl)oxy)butyl)-N-methylpentatriaconta-6,9,26,29-tetraen-18-amine as an oil (110 mg, 90%). 1H NMR (400 MHz, CDCl₃): δ 7.69-7.55 (m, 4H), 7.49-7.33 (m, 6H), 5.47-5.22 (m, 8H), 3.78-3.64 (m, 2H), 3.12-2.85 (m, 3H), 2.77 (t, J=6.4 Hz, 4H), 2.63 (br, 3H), 2.05 (q, J=6.9 Hz, 8H), 1.92-1.14 (m, 40H), 1.06 (d, J=10.5 Hz, 9H), 0.87 (t, 6H).

Desilylation of (6Z,9Z,26Z,29Z)-N-(4-((tert-butyldiphenylsilyl)oxy)butyl)-N-methylpentatriaconta-6,9,26,29-tetraen-18-amine: 4-(methyl((6Z,9Z,26Z,29Z)

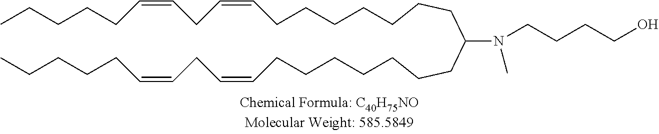 [SMILES: CCCCC/C=C\C/C=C\CCCCCCCC(CCCCCCC/C=C\C/C=C\CCCCC)N(C)CCCCO]

pentatriaconta-6,9,26,29-tetraen-18-yl)amino)butan-1-ol

To a solution of (6Z,9Z,26Z,29Z)-N-(4-((tert-butyldiphenylsilyl)oxy)butyl)-N-methylpentatriaconta-6,9,26,29-tetraen-18-amine (110 mg, 0.133 mmol) in THF (1.50 mL) was added HF-pyridine (0.100 mL, ˜70% pure, 1.11 mmol) at 0° C. The mixture was warmed to room temperature and stirred for 18 hours, quenched with water (4.00 mL) and extracted with DCM (3×4.00 mL). The combined organics were dried (Na₂SO₄) and concentrated. The residue was purified by silica chromatography (0-5% MeOH in DCM) to yield 4-(methyl((6Z,9Z,26Z,29Z)-pentatriaconta-6,9,26,29-tetraen-18-yl)amino)butan-1-ol as an oil (54.7 mg, 70%). 1H NMR (400 MHz, CDCl₃) δ 5.48-5.23 (m, 8H), 3.68 (t, J=5.5 Hz, 2H), 2.93 (br, 3H), 2.76 (t, J=6.4 Hz, 4H), 2.56 (s, 3H), 2.04 (q, J=6.8 Hz, 8H), 1.91 (s, 2H), 1.79-1.61 (m, 4H), 1.54-1.17 (m, 34H), 0.88 (t, J=6.7 Hz, 6H). LRMS (ESI+) m/z=586.

### Example 10: General Procedure for Reduction of the Ketone

Solid NaBH₄ (2 mmol) was added portion-wise to a stirred solution of ketone (2 mmol) in 95% ethanol (10 mL) at 0° C. (ice bath). After stirring at 0° C. for 1 h, the reaction was checked for completion, either by TCL (5% ether in hexanes) or—more reliably—by adding 3-4 drops of the reaction mixture to saturated aqueous NH₄Cl solution (0.5 mL), extracting with hexanes, evaporating the combined extracts to dryness, and checking the residue by ¹H NMR. Either method indicated that the reaction was complete. The reaction was quenched by careful addition of aqueous saturated NH₄Cl solution (caution should be taken due to H₂ evolution and foaming) and concentrated on the rotary evaporator to remove the ethanol. The aqueous residue was extracted with hexanes (3×10 mL). The combined extracts were passed through a plug of anhydrous Na₂SO₄ and concentrated to afford crude alcohol, which was purified by silica gel column chromatography with 5→10% v/v ethyl acetate in hexanes. The following compounds were thus obtained:

(9Z,26Z)-Pentatriaconta-9,26-dien-18-ol. H NMR δ 5.36 (m, 4H), 3.60 (m, 1H), 2.03 (m,

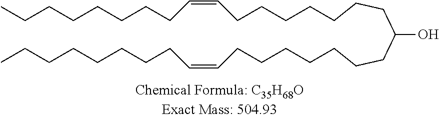 [SMILES: CCCCCCCC/C=C\CCCCCCCC(O)CCCCCCC/C=C\CCCCCCCC]

### 8H), 1.50-1.20 (m, 48H), 0.90 (t, 6H). LRMS: m/z 505 [M+H], 527 [M+Na]⁺.

(6Z,9Z,26Z,29Z)-Pentatriaconta-6,9,26,29-tetraen-18-ol. H NMR δ 5.36 (m, 8H), 3.58 (m,

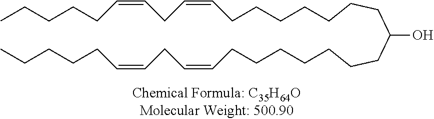 [SMILES: CCCCC/C=C\C/C=C\CCCCCCCC(O)CCCCCCC/C=C\C/C=C\CCCCC]

¹H), 2.78 (t, 4H), 2.1-1.9 (m, 8H), 1.6-1.2 (m, 36H), 0.89 (t, 6H). 13C NMR δ LRMS: m/z 501 [M+H]⁺, 523 [M+Na]⁺

6,8,26,28-Tetrathiatritriacontan-17-ol. 1H NMR δ 3.68 (s, 4H), 3.60 (m, 1H), 2.64 (m, 8H),

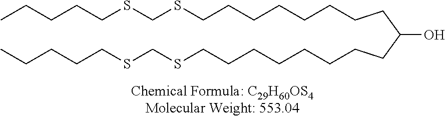 [SMILES: CCCCCSCSCCCCCCCCC(O)CCCCCCCCSCSCCCCC]

1.61 (m, 10H), 1.31 (m, 30H), 0.92 (t, 6H). 13C NMR δ 72.0, 37.5, 35.4, 31.1, 30.8, 29.6, 29.5, 29.2, 29.1, 28.9, 28.8, 25.7, 22.3, 13.9. LRMS: m/z 553 [M+H]⁺, 575 [M+Na]⁺.

### Example 11: General Procedure for Alcohol Silylation

tert-Butyl(((9Z,26Z)-pentatriaconta-9,26-dien-18-yl)oxy)diphenylsilane. A solution of tert

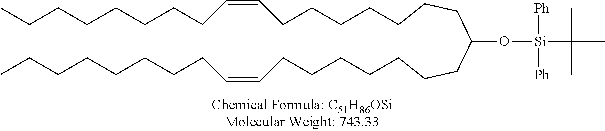 [SMILES: CCCCCCCC/C=C\CCCCCCCC(CCCCCCC/C=C\CCCCCCCC)O[Si](c1ccccc1)(c1ccccc1)C(C)(C)C]

butyldiphenylsilyl chloride (1.4 g, 5.0 mmol, 1.25 equiv) in dry CH₂Cl₂ (5 mL) was added dropwise (syringe), at room temperature, to stirred solution of (9Z,26Z)-pentatriaconta-9,26-dien-18-ol (2.0 g, 4.0 mmol, 1 equiv), triethylamine (6.0 mmol, 1.5 equiv), and 4-dimethyalminopyridine (0.2 mmol, 0.05 equiv) in dry CH₂Cl₂ (10 mL). The mixture was stirred overnight, whereupon TLC and NMR indicated complete conversion. The solution was diluted with more CH₂Cl₂ (10 mL), sequentially washed with aqueous 3% H₂SO₃ (3×10 mL) and aqueous 10% NaHCO₃ (2×10 mL), passed through a plug of anhydrous Na₂SO₄ and concentrated to give the crude product, which was carried on to the next step (ozonolysis) without purification. ¹H NMR δ 7.6-7.3 (m, 10H), 5.36 (m, 4H), 3.50 (m, 1H), 2.03 (m, 8H), 1.50-1.20 (m, 48H), 0.98 (s, 9H), 0.90 (t, 6H). LRMS: m/z 743 [M+H]⁺, 765 [M+Na]⁺.

### Example 12: Ozonolysis Reactions 8-Hydroxypentadecanedial. A Mixture of 12 mL CH₂Cl₂ and 3 mL MeOH in a 50 mL 3-Neck

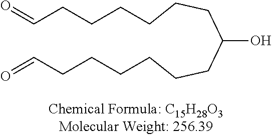 [SMILES: O=CCCCCCCC(O)CCCCCCC=O]

was placed in an RBF fitted with a football-style stirring bar, a gas inlet tube (side neck), a septum (center neck) and a loose hard plastic stopper (other side neck). An O₂ line was connected to the gas inlet, O₂ flow was started and the assembly was cooled to −78° C. The ozone generator was turned on and when the solution began to turn blue, magnetic stirring was intiated and a solution of 1 mmol of (9Z,26Z)-pentatriaconta-9,26-dien-18-ol in 3 mL CH₂Cl₂ was added dropwise via syringe. The blue color disappeared rapidly. When all of the alcohol had been added (approximately 30 min), the syringe was rinsed with 2×2 mL of CH₂Cl₂ (injected into the reaction mixture), and O₂/O₃ bubbling was continued until a blue color reappeared and persisted. The O₃ generator was turned off and O₂ bubbling was continued until the blue color disappeared. The solution was warmed to room temperature and then concentrated to about ⅓ of the original volume to remove mostly CH₂Cl₂. About 1 mL of AcOH was added, followed by Zn dust (tip of a spatulaful) and DI H₂O (ca. 1 mL). The mixture was stirred overnight, then it was concentrated (rotovap). Considerable foaming was controlled by intermittently releasing the vacuum. The white semisolid aqueous residue (Zn salts) was partitioned between DI H₂O (5 mL) and 1:1 ether/hexane (15 mL). The organic phase was passed over a 1 cm plug of silica gel deposited in a pipet. The plug was washed with Et₂O (2 mL) and the combined organic phases were evaporated to yield a nearly colorless oil. After verifying that no peroxidic agents were present (peroxide test paper), kugelrohr distillation was employed to remove nonanal (the byproduct of the reaction) from the oil. The residue from the kugelrohr was assayed by ¹H NMR and advanced to the next step crude form. 1H NMR δ 9.77 (br t, 2H), 3.58 (m, 1H), 2.42 (b t, 4H), 1.70-1.20 (m, xH). LRMS: m/z 257 [M+H]⁺, 279 [M+Na]⁺

9-((tert-butyldiphenylsilyl)oxy)heptadecane-1,17-diol. The ozonolysis of tert-butyl(((9Z,26Z)

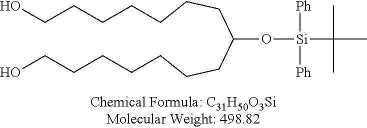 [SMILES: CC(C)(C)[Si](OC(CCCCCCCO)CCCCCCCO)(c1ccccc1)c1ccccc1]

pentatriaconta-9,26-dien-18-yl)oxy)diphen-ylsilane (p. xx) was carried out as described above, but the workup of the reaction was modified as follows. The crude residue obtained after Zn/AcOH treatment was dissolved in 95% ethanol (3 mL). Solid NaBH₄ (76 mg, 2 mmol) was added portionwise with good stirring. After 30 min, NMR analysis of the solution indicated complete conversion into the desired compound. The mixture was cooled to 0° C. and carefully treated with aqueous saturated NH₄Cl solution (caution was exercised due to H₂ that evolved), then it was evaporated under vacuum to near dryness. The residue was diluted with ethyl acetate (10 mL) and brine (2 mL). The phases were separated and the aqueous layer was extracted with more EtOAc (5 mL). The combined extracts were passed through a plug of anhydrous Na₂SO₄ and concentrated on a rotary evaporator to afford crude 9-((tert-butyldiphenylsilyl)oxy)heptadecane-1,17-diol, which was purified by column chromatography on silica gel (230-400 mesh; 30% ethyl acetate in hexanes). This afforded 0.22 g, 72% of product as a pale yellow oil. H NMR δ 7.69 (d, 4H), 7.40 (m, 6H), 4.59 (t, 2H), 3.88 (m, 2H), 3.72 (t, ¹H), 3.65 (t, 4H), 1.40 (m, 10H), 1.23 (m, 20H), 1.06 (s, 9H). 13C NMR δ 135.9, 134.8, 129.35, 127.4, 73.2, 63.1, 36.3, 32.8, 29.6, 29.6, 29.5, 29.3, 27.1, 25.7, 24.8, 19.4. LRMS: m/z 521 [M+Na]⁺

### Example 13: Procedure for Thioacetalization of Lipid Precursors

1,1,17,17-Tetrakis(octylthio)heptadecan-9-ol. A stirred solution of 1-octanethiol (4.2 mol) and

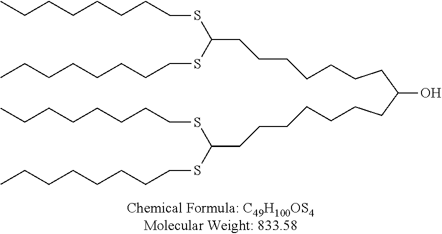 [SMILES: CCCCCCCCSC(CCCCCCCC(O)CCCCCCCC(SCCCCCCCC)SCCCCCCCC)SCCCCCCCC]

8-hydroxypentadecanedial (1 mmol) in ether (3 mL) was cooled to 0° C. and treated with 1 drop of conc. H₂SO₄. The mixture was stirred for 1 h and allowed to reach room temperature, whereupon NMR indicated that reaction was complete. The mixture was diluted with 1:1 ether-hexane (5 mL) and washed with 10% aq. NaOH solution, then it was passed over a plug of anhydrous Na₂SO₄ and evaporated. The oily residue was purified by column chromatography on silica gel (30 g, solvent) to afford pure thioacetal. H NMR δ 3.75 (t, 2H), 3.60 (m, 1H), 2.74-2.49 (m, 8H), 1.69-1.20 (m, 76H), 0.90 (t, 12H). 13C NMR δ 71.6, 52.1, 36.2, 34.1, 32.3, 30.1, 29.5, 29.44, 29.40, 29.2, 29.1, 29.0, 27.5, 25.3, 22.6, 14.1. LRMS: m/z 855 [M+Na]⁺

### Example 14: General Procedure for Alcohol (4-Dimethylamino)Butanoylation

A solution of an alcohol (1 mmol, 1.0 equiv), 4-dimethylaminobutyric acid hydrochloride (1.2 mmol, 1.2 equiv), diisopropylethylamine (1.5 mmol, 1.5 equiv), and DMAP (0.1 mmol, 0.1 equiv) in dry CH₂Cl₂ (3 mL) was stirred at room temperature for 5 minutes prior to the addition of EDCI (1.5 mmol, 1.5 equiv). The mixture was stirred overnight at room temperature, under argon, whereupon TLC (5% MeOH in CH₂Cl₂) and ¹H NMR indicated that the reaction had completed. The solution was diluted with more CH₂Cl₂ (10 mL) and sequentially washed with aqueous saturated NaHCO₃(5 mL) and water (10 mL). The organic phase was passed over a plug of anhydrous Na₂SO₄ and concentrated in vacuo. The residue of crude product was purified by flash column chromatography with 3% v/v MeOH in CH₂Cl₂, containing 0.1% NEt₃.

The following compounds were thus obtained:

(6Z,9Z,26Z,29Z)-Pentatriaconta-6,9,26,29-tetraen-18-yl 4-(dimethylamino)butanoate.

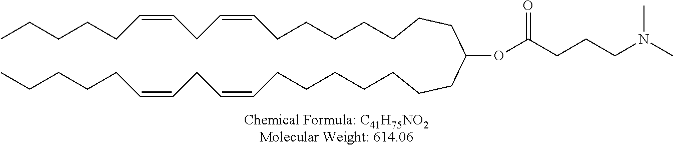 [SMILES: CCCCC/C=C\C/C=C\CCCCCCCC(CCCCCCC/C=C\C/C=C\CCCCC)OC(=O)CCCN(C)C]

¹H NMR δ 5.33 (m, 8H), 4.85 (m, 1H), 2.75 (t, 4H), 2.40 (m, 2H), 2.34 (t, 2H), 2.20 (s, 6H), 2.02 (m, 8H), 1.77 (m, 2H), 1.50 (m, 6H), 1.39-1.17 (m, 36H), 0.87 (t, 6H). 13C NMR δ 173.3, 130.1, 130.0, 127.9, 127.8, 74.1, 59.0, 45.4, 34.1, 32.4, 31.5, 29.7, 29.6, 29.5, 29.4, 29.33, 29.30, 29.2, 27.15, 27.14, 25.3, 23.1, 14.8. LRMS: m/z 614 [M+H]⁺.

1,1,17,17-Tetrakis(octylthio)heptadecan-9-yl 4-(dimethylamino)butanoate

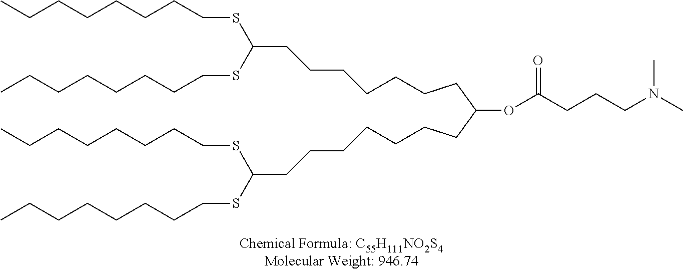 [SMILES: CCCCCCCCSC(CCCCCCCC(CCCCCCCC(SCCCCCCCC)SCCCCCCCC)OC(=O)CCCN(C)C)SCCCCCCCC]

¹H NMR δ 4.87 (m, 1H), 3.72 (t, 2H); 2.6 (m, 8H), 2.33 (m, 4H), 2.27 (s, 6H), 1.77 (m, 6H); 1.53 (m, 16H), 1.44-1.20 (m, 60H), 0.88 (t, 12H). C NMR δ 173.3, 74.3, 58.8, 52.1, 45.2, 36.2, 34.1, 32.3, 31.8, 30.1, 29.5, 29.44, 29.40, 29.2, 29.1, 29.0, 27.5, 25.3, 23.0, 22.6, 14.1. LRMS: m/z 946 [M+H]⁺. HRMS: calc. for C₅₅H₁₁₂NO₂S₄[M+1]⁺ m/z 946.7542; found 946.7552.

1,1,17,17-tetrakis(butylthio)heptadecan-9-yl 4-(dimethylamino)butanoate

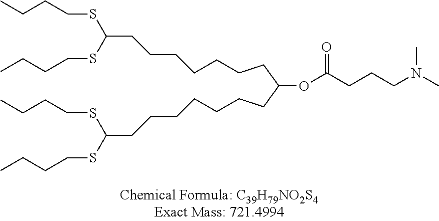 [SMILES: CCCCSC(CCCCCCCC(CCCCCCCC(SCCCC)SCCCC)OC(=O)CCCN(C)C)SCCCC]

¹H NMR (400 MHz, CDCl₃): δ 4.84 (p, J=6.3 Hz, 1H), 3.72 (t, J=7.0 Hz, 2H), 2.70-2.50 (m, 8H), 2.45 (s, 6H), 2.35 (t, J=7.1 Hz, 2H), 1.90 (p, J=7.2 Hz, 2H), 1.75 (dt, J=9.4, 7.1 Hz, 4H), 1.67-1.17 (m, 42H), 0.91 (t, J=7.3 Hz, 12H). LRMS (ESI+) m/z=722 [M+H]⁺.

6,8,26,28-tetrathiatritriacontan-17-yl 4-(dimethylamino)butanoate

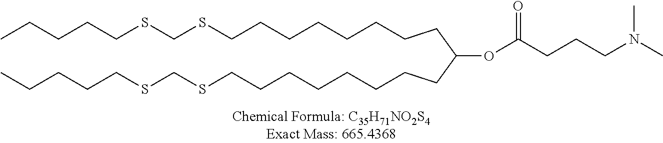 [SMILES: CCCCCSCSCCCCCCCCC(CCCCCCCCSCSCCCCC)OC(=O)CCCN(C)C]

¹H NMR δ 4.85 (m, 1H), 3.65 (s, 4H), 2.62 (m, 8H), 2.30 (m, 10H), 1.77 (m, 2H), 1.57 (m, 12H), 1.31 (m, 28H), 0.89 (t, 6H). 13C NMR δ 172.09, 75.37, 57.23, 43.07, 35.52, 34.10, 31.18, 31.07, 30.93, 29.53, 29.49, 29.24, 29.14, 28.95, 28.87, 25.45, 22.40, 19.85, 14.09. LRMS: m/z 666 [M+H]⁺.

### Example 15: General Procedure for Succinoylation of the Alcohol

A solution of alcohol (1 mmol), succinic or glutaric anhydride (1.1 mmol), and 4-dimethylaminopyridine (0.05 mmol) in pyridine (2 mL) was heated to 90° C. overnight, whereupon ¹H NMR indicated that all the alcohol had been converted into the desired product. The mixture was carefully poured into a separatory funnel containing 2 N aqueous HCl (10 mL) and ether (15 mL). The layers were separated and the aqueous phase was extracted with more ether (5 mL). The combined extracts were washed with brine (5 mL), filtered through a plug of anhydrous Na₂SO₄ and evaporated to dryness. The following compounds were thus obtained:

4-Oxo-4-(((9Z,26Z)-pentatriaconta-9,26-dien-18-yl)oxy)butanoic acid

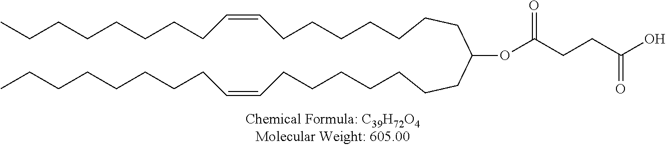 [SMILES: CCCCCCCC/C=C\CCCCCCCC(CCCCCCC/C=C\CCCCCCCC)OC(=O)CCC(=O)O]

¹H NMR δ 5.36 (m, 4H), 4.90 (m, 1H), 2.74-2.60 (m, 4H), 2.03 (m, 8H), 1.53 (m, 4H), 1.41-1.20 (m, 44H), 0.90 (t, 6H). LRMS (negative ion mode): m/z 603.

5-(hentriacontan-16-yloxy)-5-oxopentanoic acid

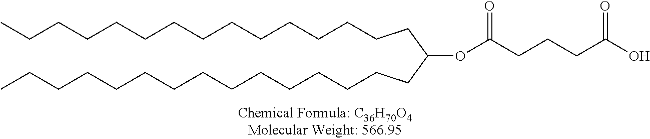 [SMILES: CCCCCCCCCCCCCCCC(CCCCCCCCCCCCCCC)OC(=O)CCCC(=O)O]

¹H NMR δ4.77 (m, 1H), 2.40-2.28 (NM, 4H), 2.13 (m, 2H), 1.53-1.20 (m, 56H), 0.86 (t, 6H). LRMS (neg. ion mode): m/z 565 [M−H]

The foregoing examples are illustrative only. That is, various alterations can be made without departing from the scope of certain aspects of the invention as described herein.

1. A method for producing an ionizable lipid, the method comprising:
(i) providing a starting material that is a fatty ester;
(ii) reacting the fatty ester in a Claisen condensation reaction in the presence of a catalyst selected from AlCl₃, GaCl₃, TiCl₄, ZrCl₄, HfCl₄ or SnCl₄, the Claisen condensation employing a weak base selected from tributylamine or triethylamine and carried out at a temperature of between−10 and 60 degrees Celsius to produce a ketoester,
wherein the fatty ester has a structure of Formula I: 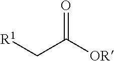
Formula I, wherein R¹ is a linear or branched alkyl group or alkenyl group having from 4 to carbon atoms, and wherein the alkyl or alkenyl group optionally has (i) 0 to 4 heteroatoms, (ii) 0 to 5 C═C double bonds of E or Z geometry, and/or (iii) substituents selected from one or more of OH, O-alkyl, S-alkyl, and N(alkyl)₂ bonded to a carbon atom thereof, and R′ is an alkyl group having up to 5 carbon atoms;
and wherein the ketoester has a structure of Formula II: 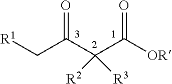
Formula II,
wherein R² is a linear or branched alkyl or alkenyl group having from 4 to 30 carbon atoms, and wherein the alkyl or alkenyl groups optionally incorporate (i) 0 to 4 heteroatoms, (ii) 0 to 5 C═C double bonds of E or Z geometry, and/or (iii) substituents selected from OH, O-alkyl, S-alkyl, and N(alkyl)₂ bonded to a carbon atom thereof;
R³ is H, or a linear or branched alkyl of alkenyl group having from 4 to 30 carbon atoms, that optionally incorporates (i) 0 to 4 heteroatoms, (ii) 0 to 5 C═C double bonds of E or Z geometry, and/or (iii) substituents selected from OH, O-alkyl, S-alkyl, and N(alkyl)₂ bonded to a carbon atom;
(iii) reacting the ketoester produced in step-(i) step (ii) under conditions to produce a ketone from the ketoester in one or more steps via a hydrolysis and decarboxylation of the ketoester with sequential base and acid addition,
wherein the ketone has a structure of Formula III, 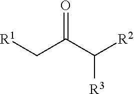
Formula III; and
(iv) synthesizing the ionizable lipid from the ketone thereof using one or more synthesis steps resulting in an addition of an ionizable head group moiety to (a) the ketone; or (b) an alcohol produced from an optional reduction of the ketone to produce the alcohol, thereby producing the ionizable lipid.

2. The method of claim 1, wherein the ketone is subjected to the reduction in step (iv) to produce the alcohol.

3. The method of claim 2, wherein the reduction in step (iv) comprises subjecting the ketone to a reducing agent that is sodium borohydride.

4. The method of claim 1, wherein the catalyst is TiCl₄.

5. The method of claim 1, wherein in the hydrolysis and decarboxylation, the base is an aqueous strong base and the acid is an aqueous strong acid, and wherein the aqueous strong acid is added to a resultant solution formed upon an addition of the aqueous strong base to the ketoester.

6. The method of claim 5, wherein the strong base is selected from lithium hydroxide, sodium hydroxide, potassium hydroxide, calcium hydroxide, barium hydroxide and tetraalkylammonium hydroxides and the strong acid is selected from hydrochloric acid, sulfuric acid and phosphoric acid.

7. The method of claim 5, wherein the aqueous strong base is sodium hydroxide, and the aqueous strong acid is hydrochloric acid.

8. The method of claim 1, wherein the hydrolysis and decarboxylation of the ketoester further comprises a step of heating.

9. The method of claim 1, wherein the fatty ester of step (i) is obtained from a synthesis scheme comprising a step of ozonolysis to cleave a double bond in an alkyl chain of a precursor fatty ester to produce an aldehyde derivative of the precursor fatty ester.

10. The method of claim 1, wherein the one or more steps resulting in an addition of an ionizable head group moiety to the ketone or alcohol comprises 1 to 5 steps.

11. The method of claim 1, wherein the ionizable lipid produced in step (iv) comprises a linker region.

12. The method of claim 1, wherein the fatty ester is a methyl ester or ethyl ester.

13. The method of claim 1, wherein the fatty ester is a methyl ester selected from methyl linoleate, methyl linolenate, methyl myristoleate, methyl palmitoleate, methyl myristate, methyl palmitate, methyl stearate, methyl 9-(((octylthio)methyl)thio)nonanoate and methyl 9,9-bis(octylthio)nonanoate.

14. The method of claim 1, further comprising an addition of an R³ alkyl group to the ketoester prior to the hydrolysis and decarboxylation of the ketoester.

15. A method for preparing a drug delivery vehicle comprising (a) producing an ionizable lipid using the method of claim 1; and (b) formulating the ionizable lipid so produced in the drug delivery vehicle.

16. The method of claim 15, wherein the drug delivery vehicle is a lipid nanoparticle.

17. The method of claim 15, wherein the step of formulating comprises admixing a therapeutic agent or prodrug, with the ionizable lipid.

18. The therapeutic agent in claim 17, wherein a nucleic acid, peptide, ribonucleoprotein, or protein is admixed with the ionizable lipid and wherein the drug delivery vehicle comprises the nucleic acid, peptide, ribonucleoprotein or protein.

19. The method of claim 17, further comprising admixing the therapeutic agent or prodrug with additional lipids.

20. The method of claim 19, wherein the additional lipids are structural lipids or a sterol.

21. The method of claim 19, wherein the additional lipids are ionizable lipids.

22. (canceled)
